SEQUENCE LISTING (44 sequences)

SEQ ID NO: 1 - protein, 15 aa, synthetic construct
  REGION 1..15: note = Synthetic polypeptide
  VARIANT 15: note = X = Lys or AlexaFluor or DBCO or azide
  MOD_RES 5: note = X = beta-beta'-di-phenyl-Ala
SQFDXCTRRLQSGGX

SEQ ID NO: 2 - protein, 14 aa, synthetic construct
  REGION 1..14: note = Synthetic polypeptide
  MOD_RES 4: note = X = beta-beta'-di-phenyl-Ala
  MOD_RES 7: note = X = n-octylthiol-substituted-Arg
QFDXSTXRLKSGGK

SEQ ID NO: 3 - protein, 17 aa, synthetic construct
  REGION 1..17: note = Synthetic polypeptide
  MOD_RES 5: note = X = beta-beta'-di-phenyl-Ala
SQFDXSTRRLQSSGCGK

SEQ ID NO: 4 - protein, 15 aa, synthetic construct
  REGION 1..15: note = Synthetic polypeptide
  MOD_RES 5: note = X = beta-beta'-di-phenyl-Ala
SQFDXCTRRLQSGGK

SEQ ID NO: 5 - protein, 14 aa, synthetic construct
  REGION 1..14: note = Synthetic polypeptide
HTFPCTLQSSGLYS

SEQ ID NO: 6 - protein, 14 aa, synthetic construct
  REGION 1..14: note = Synthetic polypeptide
HPFPCTLQSSGLYS

SEQ ID NO: 7 - protein, 14 aa, synthetic construct
  REGION 1..14: note = Synthetic polypeptide
HAFPCTLQSSGLYS

SEQ ID NO: 8 - protein, 14 aa, synthetic construct
  REGION 1..14: note = Synthetic polypeptide
HSFPCTLQSSGLYS

SEQ ID NO: 9 - protein, 14 aa, synthetic construct
  REGION 1..14: note = Synthetic polypeptide
HTDPCVLQSSGLYS

SEQ ID NO: 10 - protein, 14 aa, synthetic construct
  REGION 1..14: note = Synthetic polypeptide
HTGPCVLQSSGLYS

SEQ ID NO: 11 - protein, 14 aa, synthetic construct
  REGION 1..14: note = Synthetic polypeptide
HTTPCVLQSSGLYS

SEQ ID NO: 12 - protein, 14 aa, synthetic construct
  REGION 1..14: note = Synthetic polypeptide
HTEPCVLQSSGLYS

SEQ ID NO: 13 - protein, 14 aa, synthetic construct
  REGION 1..14: note = Synthetic polypeptide
HTHPCVLQSSGLYS

SEQ ID NO: 14 - protein, 14 aa, synthetic construct
  REGION 1..14: note = Synthetic polypeptide
HTAPCVLQSSGLYS

SEQ ID NO: 15 - protein, 14 aa, synthetic construct
  REGION 1..14: note = Synthetic polypeptide
HTFPCILQSSGLYS

SEQ ID NO: 16 - protein, 14 aa, synthetic construct
  REGION 1..14: note = Synthetic polypeptide
HTFPCTLQSSGLYS

SEQ ID NO: 17 - protein, 14 aa, synthetic construct
  REGION 1..14: note = Synthetic polypeptide
HTFPCLLQSSGLYS

SEQ ID NO: 18 - protein, 14 aa, synthetic construct
  REGION 1..14: note = Synthetic polypeptide
HTFPCSLQSSGLYS

SEQ ID NO: 19 - protein, 14 aa, synthetic construct
  REGION 1..14: note = Synthetic polypeptide
HTFPCLLQSSGLYS

SEQ ID NO: 20 - protein, 14 aa, synthetic construct
  REGION 1..14: note = Synthetic polypeptide
HTFPCFLQSSGLYS

SEQ ID NO: 21 - protein, 14 aa, synthetic construct
  REGION 1..14: note = Synthetic polypeptide
HTFPCELQSSGLYS

SEQ ID NO: 22 - protein, 16 aa, synthetic construct
  REGION 1..16: note = Synthetic polypeptide
SQFDFCTRRLQSGGSK

SEQ ID NO: 23 - protein, 16 aa, synthetic construct
  REGION 1..16: note = Synthetic polypeptide
  SITE 5: note = (Ph)2
SQFDACTRRLQSGGSK

SEQ ID NO: 24 - protein, 16 aa, synthetic construct
  REGION 1..16: note = Synthetic polypeptide
  SITE 5: note = (Ph)2
SQFDASTRRLQSGGSK

SEQ ID NO: 25 - protein, 12 aa, synthetic construct
  REGION 1..12: note = Synthetic polypeptide
SQFDLCTRRLQS

SEQ ID NO: 26 - protein, 16 aa, synthetic construct
  REGION 1..16: note = Synthetic polypeptide
  MOD_RES 1: note = ACETYLATION
  SITE 5: note = (Ph)2
SQFDASTRRLQSGGSK

SEQ ID NO: 27 - protein, 16 aa, synthetic construct
  REGION 1..16: note = Synthetic polypeptide
  MOD_RES 1: note = ACETYLATION
SQFDFCTRRLQSGGSK

SEQ ID NO: 28 - protein, 16 aa, synthetic construct
  REGION 1..16: note = Synthetic polypeptide
  MOD_RES 1: note = ACETYLATION
  SITE 5: note = (Ph)2
SQFDACTRRLQSGGSK

SEQ ID NO: 29 - protein, 234 aa, synthetic construct
  REGION 1..234: note = Synthetic polypeptide
METDTLLLWVLLLWVPGSTGDIQMTQSPILLSASVGDRVTITCRASQDVNTAVAWYQQRTNGSPRLLIYSASFLYSGVPSRFSGSRSGTDFTLTISSLQPEDEADYYCQQHYTTPPTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 469 aa, synthetic construct
  REGION 1..469: note = Synthetic polypeptide
MKCSWVIFFLMAVVTGVNSEVQLVESGGGLVQPGGSLRLSCAASGFNIKDTYIHWVRQSPGKGLEWVARIYPTNGYTRYADSVKGRFTISADTSKNTAYLQMNSLRAEDTAIYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 469 aa, synthetic construct
  REGION 1..469: note = Synthetic polypeptide
MKCSWVIFFLMAVVTGVNSEVQLVESGGGLVQPGGSLRLSCAASGFNIKDTYIHWVRQSPGKGLEWVARIYPTNGYTRYADSVKGRFTISADTSKNTAYLQMNSLRAEDTAIYYCSRWGGDGFYAMDYWGQGTLVTVSSASTKGPSVFPLAPSSKSTSGGTAALGCLVKDYFPEPVTVSWNSGALTSGVHTFPCVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 215 aa, synthetic construct
  REGION 1..215: note = Synthetic polypeptide
DIQMTQSPILLSASVGDRVTITCRASQSIRSYLNWYQQRTNGSPRLLIYAASSLQSGVPSRFSGSGSGTDFTLTINSLQPDDEADYYCQQTYSNPPITFGAGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 231 aa, synthetic construct
  REGION 1..231: note = Synthetic polypeptide
EVHLVESGGGLVQPGRSLRLSCAASGFTFDDYTMHWVRQAPGKGLEWVSDISWNSGSIGYADSVKGRFTVSRDNAKNSLYLQMNSLRGEDTAIYYCAKDMSGYGHYGKYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 34 - protein, 214 aa, synthetic construct
  REGION 1..214: note = Synthetic polypeptide
EIVMTQSPILLSVSPGERAILSCRASQNINSNLAWYQQRTNGSPRLLIYGASTRATGVPARFSGSGSGTEFTLTIRSLQPEDEADYYCQQYYNWPITFGAGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 230 aa, synthetic construct
  REGION 1..230: note = Synthetic polypeptide
EVQLVESGGGLVQPGRSLRLSCVASGFTFADFTMHWVRQAPGKGLEWVSGISWNSNSIDYADSVKGRFTISRDNAKKSLFLQMSSLRAEDTAIYYCVKDRSGYSRFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 36 - protein, 215 aa, synthetic construct
  REGION 1..215: note = Synthetic polypeptide
DIQMTQSPILLSASVGDRVTITCRASQSISSYLNWYQQRTNGSPRLLIYAASSLQSGVPSRFSGSGSGTDFTLTISSLQPEDEADYYCQQSYSTPPITFGAGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 231 aa, synthetic construct
  REGION 1..231: note = Synthetic polypeptide
EVQLVESGGGLVQPGRSLRLSCAASGFTFADYTMHWVRQAPGKGLEWVSDISWNSGSIAYADSVKGRFTISRDNAKNSLYLQMNSLRTEDTAIYYCAKDSRGYGHYKYLGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 38 - protein, 256 aa, synthetic construct
  REGION 1..256: note = Synthetic polypeptide
SQFDLCTRRLQSGSGSGSVSKGEELFTGVVPILVELDGDVNGHKFSVRGEGEGDATNGKLTLKFISTTGKLPVPWPTLVTTLTYGVQSFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITHGMDELYK

SEQ ID NO: 39 - protein, 256 aa, synthetic construct
  REGION 1..256: note = Synthetic polypeptide
VSKGEELFTGVVPILVELDGDVNGHKFSVRGEGEGDATNGKLTLKFISTTGKLPVPWPTLVTTLTYGVQSFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITHGMDELYKGSGSGSSQFDLCTRRLQS

SEQ ID NO: 40 - protein, 274 aa, synthetic construct
  REGION 1..274: note = Synthetic polypeptide
SQFDLCTRRLQSGSGSGSVSKGEELFTGVVPILVELDGDVNGHKFSVRGEGEGDATNGKLTLKFISTTGKLPVPWPTLVTTLTYGVQSFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITHGMDELYKGSGSGSSQFDLCTRRLQS

SEQ ID NO: 41 - protein, 74 aa, synthetic construct
  REGION 1..74: note = Synthetic polypeptide
SQFDLCTRRLQSGGGSVDNKFNKEMRNAYWEIALLPNLNNQQKRAFIRSLYDDPSQSANLLAEAKKLNDAQAPK

SEQ ID NO: 42 - protein, 165 aa, synthetic construct
  REGION 1..165: note = Synthetic polypeptide
MKKIWLALAGLVLAFSASASQFDLCTRRLQSGGGSEVQLVESGGELVQAGGSLRLSCAASGLTFSSYNMGWFRRAPGKEREFVASITWSGRDTFYADSVKGRFTISRDNAKNTVYLQMSSLKPEDTAVYYCAANPWPVAAPRSGTYWGQGTQVTVSSGSHHHHHH

SEQ ID NO: 43 - protein, 163 aa, synthetic construct
  REGION 1..163: note = Synthetic polypeptide
MKKIWLALAGLVLAFSASAEVQLVESGGELVQAGGSLRLSCAASGLTFSSYNMGWFRRAPGKEREFVASITWSGRDTFYADSVKGRFTISRDNAKNTVYLQMSSLKPEDTAVYYCAANPWPVAAPRSGTYWGQGTQVTVSSGSHHHHHHGGSQFDLCTRRLQS

SEQ ID NO: 44 - protein, 12 aa, synthetic construct
  REGION 1..12: note = Synthetic polypeptide
CQFDLSTRRLKC

(19) United States
(12) Patent Application Publication
(10) Pub. No.: US 2025/0066418 A1
(43) Pub. Date: Feb. 27, 2025

(54) CYSTEINE PEPTIDE-ENABLED ANTIBODIES

(71) Applicant: City of Hope, Duarte, CA (US)

(72) Inventors: John C. Williams, Monrovia, CA (US); Krzysztof Bzymek, Pasadena, CA (US); Yuelong Ma, Duarte, CA (US); David Horne, Altadena, CA (US); Jeremy King, Duarte, CA (US)

(21) Appl. No.: 18/608,790

(22) Filed: Mar. 18, 2024

Related U.S. Application Data
(63) Continuation of application No. 16/467,450, filed on Jun. 6, 2019, now abandoned.
(60) Provisional application No. 62/531,825, filed on Jul. 12, 2017.
(60) Provisional application No. 62/430,848, filed on Dec. 6, 2016.

(51) Int. Cl. C07K 7/06; A61K 47/68; C07K 7/08; C07K 16/28; C07K 16/32

(52) U.S. Cl. CPC C07K 7/06; A61K 47/68031; A61K 47/68033; A61K 47/6851; A61K 47/6889; C07K 7/08; C07K 16/283; C07K 16/32; C07K 2317/55; C07K 2317/624

(57) ABSTRACT

Provided herein are functionalized monoclonal antibodies (mAbs) including antibody fragments covalently linked to a peptide compound through a disulfide linkage. The disulfide linkage is between a cysteine in the Fab region of the antibody or fragment thereof and a thiol moiety of a side chain amino acid of the peptide compound. The covalently formed complexes including provided herein form highly stable and versatile drug delivery and diagnostic compositions.

### CROSS-REFERENCES TO RELATED APPLICATIONS

This application is a continuation of U.S. application Ser. No. 16/467,450 filed Jun. 6, 2019 (now abandoned), which is a U.S. 371 National Phase Application of International Application No. PCT/US2017/064969 filed Dec. 6, 2017, which application claims the benefit of U.S. Provisional Application No. 62/430,848, filed Dec. 6, 2016, and U.S. Provisional Application No. 62/531,825, filed on Jul. 12, 2017, both of which are incorporated herein by reference in their entirety and for all purposes.

### SEQUENCE LISTING

The Sequence Listing written in file 2024-11-13 Revised_Sequence_Listing_ST26 048440-627C01US.xml, created Nov. 13, 2024, 64,565 bytes, is hereby incorporated by reference.

### BACKGROUND OF THE INVENTION

Due to their specificity and favorable pharmacokinetics and pharmacodynamics, there have been substantial efforts to arm monoclonal antibodies (mAbs) either with potent cytotoxins or biologics to enhance their therapeutic efficacy or with radionuclides to image disease. These methods are limited by available chemistries of the parental mAb and/or require extensive protein engineering. Further, there is a need to functionalize antibodies through covalent bonds. The compositions and methods provided herein address these and other needs in the art.

### SUMMARY OF THE INVENTION

In a first aspect, there is provided a covalent complex including: (i) an antigen binding domain including: (1) a central hole enclosed by the heavy chain variable (VH) region, the light chain variable (VL) region, the heavy chain constant (CH1) region and the light chain constant (CL) region of the antigen binding domain between a first cavity and a second cavity; and (2) a non-CDR peptide binding region including: (a) the first cavity lined by a first set of amino acid residues of the VH, VL, CH1, and CL regions of the antigen binding domain; (b) the second cavity lined by a second set of amino acid residues of the VH, VL, CH1, and CL regions of the antigen binding domain; or (c) a hole region enclosing the hole between the first cavity and the second cavity, the hole region lined by a third set of amino acid residues of the VH, VL, CH1, and CL regions of the antigen binding domain. The non-CDR peptide binding region includes a first cysteine; and (ii) a peptide compound including a thiol side chain amino acid covalently bound to the antigen binding domain through a disulfide linkage between the first cysteine and the thiol side chain amino acid.

In another aspect, a peptide compound of formula:

R¹-X0-X1-X2-X3-X4-X5-X6-X7-X8-X9-X10-X11-X12-R² (I)

is provided. In formula (I), X0 is Ser or null. X1 is Ser, Cys, Gly, β-alanine, diaminopropionic acid, β-azidoalanine or null. X2 is Gln or null. X3 is Phe, Tyr, β,β′-diphenyl-Ala, His, Asp, 2-bromo-L-phenylalanine, 3-bromo-L-phenylalanine, 4-bromo-L-phenylalanine, Asn, Gln, a modified Phe, a hydratable carbonyl-containing residue or a boronic acid-containing residue. X4 is Asp or Asn. X5 is Leu, β,β′-diphenyl-Ala, Phe, Trp, Tyr, a non-natural analog of phenylalanine, tryptophan, or tyrosine, a hydratable carbonyl-containing residue or a boronic acid-containing residue. X6 is Cys, protected Cys or Ser. X7 is Cys, protected Cys, Thr, or Ser. X8 is protected Arg, Arg, Ala, or an amino acid comprising a side chain of the formula -L^{3A}-L^{3B}-R³, wherein L^{3A} is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, L^{3B} is a chemical linker and R³ is a steric hindering chemical moiety. X9 is Cys, protected Cys, Arg or Ala. X10 is Leu, Gln, Glu, β,β′-diphenyl-Ala, Phe, Trp, Tyr; a non-natural analog of phenylalanine, tryptophan, or tyrosine, a hydratable carbonyl-containing residue, or a boronic acid-containing residue. X11 is the Cys, protected Cys, Gln, Lys or Arg. X12 is Ser, Cys, Gly, 7-aminoheptanoic acid, β-alanine, diaminopropionic acid, propargylglycine, isoaspartic acid, or null. R¹ is null, -L^{10A}-L^{10B}-R¹⁰, an amino acid peptide sequence optionally substituted with -L^{10A}-L^{10B}-R₁₀. R² is null, -L^{20A}-L^{20B}-R²⁰, an amino acid peptide sequence optionally substituted with -L^{20A}-L^{20B}-R²⁰. L^{10A}, L^{10B}, L^{20A}, L^{20B} are independently a bond, a peptidyl linker, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. R¹⁰ and R²⁰ are independently a reactive moiety, a diagnostic moiety, a therapeutic moiety or a detectable moiety. X1 and X12 are optionally joined together to form a cyclic peptidyl moiety. R¹ and X11 are optionally joined together to form a cyclic peptidyl moiety.

In another aspect, a peptide compound of formula:

R¹-X0-X1-X2-X3-X4-X5-X6-X7-X8-X9-X10-X11-X12-X13-X14-X15-R² (II)

is provided. In formula (II), X0 is Ser or null. X1 is Ser, Cys, Gly, β-alanine, diaminopropionic acid, β-azidoalanine, or null. X2 is Gln or null. X3 is Phe, Tyr, β,β′-diphenyl-Ala, His, Asp, 2-bromo-L-phenylalanine, 3-bromo-L-phenylalanine, 4-bromo-L-phenylalanine, Asn, Gln, a modified Phe, a hydratable carbonyl-containing residue, or a boronic acid-containing residue. X4 is Asp or Asn. X5 is Leu, β,β′-diphenyl-Ala, Phe, Trp, Tyr, a non-natural analog of phenylalanine, tryptophan, or tyrosine, a hydratable carbonyl-containing residue, or a boronic acid-containing residue. X6 is Ser. X7 is Cys, protected Cys, Thr, or Ser. X8 is Arg, Ala, or an amino acid comprising a side chain of the formula -L^{3A}-L^{3B}-R³, wherein L^{3A} is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, L^{3B} is a chemical linker and R³ is a steric hindering chemical moiety. X9 is Cys, protected Cys, Arg or Ala. X10 is Leu, Gln, Glu, β,β′-diphenyl-Ala, Phe, Trp, Tyr, a non-natural analog of phenylalanine, tryptophan, or tyrosine, a hydratable carbonyl-containing residue, or a boronic acid-containing residue. X11 is Cys, protected Cys, Gln, Lys or Arg. X12 is Ser, Cys, Gly, 7-aminoheptanoic acid, β-alanine, diaminopropionic acid, propargylglycine, isoaspartic acid, or null. X13 is Gly or Ser. X14 and X15 are independently Gly, Ser, Ala, Cys or protected Cys. R¹ is null, -L^{10A}-L^{10B}-R¹⁰, an amino acid peptide sequence optionally substituted with -L^{10A}-L^{10B}-R₁₀. R² is null, -L^{20A}-L^{20B}-R²⁰, an amino acid peptide sequence optionally substituted with -L^{20A}-L^{20B}-R²⁰. L^{10A} L^{10B}, L^{20A}, L^{20B} are independently a bond, a peptidyl linker, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. R¹⁰ and R²⁰ are independently a reactive moiety, a diagnostic moiety, a therapeutic moiety or a detectable moiety. X1 and X12 are optionally joined together to form a cyclic peptidyl moiety.

In another aspect, an antigen binding domain is provided. The antigen binding domain includes: (1) a central hole enclosed by the heavy chain variable (VH) region, the light chain variable (VL) region, the heavy chain constant (CH1) region and the light chain constant (CL) region of the antigen binding domain between a first cavity and a second cavity; and (2) a non-CDR peptide binding region including: (a) the first cavity lined by a first set of amino acid residues of the VH, VL, CH1, and CL regions of the antigen binding domain, wherein the first set of amino acid residues includes a cysteine at a position corresponding to Kabat position 102, 142 or 143 of the VL region; (b) the second cavity lined by a second set of amino acid residues of the VH, VL, CH1, and CL regions of the antigen binding domain, wherein the second set of amino acid residues includes a cysteine at a position corresponding to Kabat position 208 or 158 of the VH region; or (c) a hole region enclosing the hole between the first cavity and the second cavity, the hole region lined by a third set of amino acid residues of the VH, VL, CH1, and CL regions of the antigen binding domain, wherein the third set of amino acid residues includes a cysteine at a position corresponding to Kabat position 174 or 175 of the VH region.

### BRIEF DESCRIPTION OF THE DRAWINGS

FIG. 1. Crystal structure of a trastuzumab meditope-enabled antibody (memAb) with a meditope bound within the meditope binding pocket.

FIG. 2A-2B. Cartoon and electron density map depiction of the formation of a disulfide bond between cysteine (Cys) residues of the Cys-modified (Ser6Cys) meditope (Cys-meditope) and the Cys-modified (Ala175Cys) trastuzumab meditope-enabled Fragment antigen-biding domain (Cys-meFab). FIG. 2A) Cartoon depicting the reaction between a linear Cys-meditope and Cys-meFab. The Cys residues of the meFab and meditope form a disulfide bond resulting in covalent linkage of the meFab and meditope. FIG. 2B) Electron density map and atomic structure of the crystalized Cys-meditope/Cys-meFab complex. Oval and arrows indicate the formation of a disulfide bond.

FIG. 3A-3H. Surface plasmon resonance (SPR) sensograms and crystal structures of meditope peptide variants binding to immobilized memAb variants. FIG. 3A) Close-up of Leu5 of original meditope bound to original trastuzumab memAb. FIG. 3B) Close-up of long 5-diphenylalanine meditope binding to original trastuzumab memAB. FIG. 3C) Close-up of 183 in the light chain of the original trastuzumab memAb. FIG. 3D) Mutation of 183 to glutamate and its juxtaposition to Arg9 of the meditope. FIG. 3E-H) SPR sensograms collected at 37° C. FIG. 3E) SPR sensogram and binding affinity of original meditope and original trastuzumab memAb. FIG. 3F) SPR sensogram and binding affinity of 5-diphenylalanine meditope and original trastuzumab memAb. A ˜25 fold increase in affinity occurs by replacing Leu5 with 5-diphenylalanine in the meditope. FIG. 3G) SPR sensogram and binding affinity of original meditope and mutated 183 trastuzumab memAb. A ˜25 increase in affinity is observed with 183 mutated to glutamate. FIG. 3H) SPR sensogram and binding affinity of 5-diphenylalanine meditope and mutated trastuzumab memAb. The combination produces an 1160 fold increase in the overall affinity.

FIG. 4A-4B. Disulfide bonds form between Cys-meditopes and trastuzumab Cys-meFabs with the reaction going to completion in approximately 3 hours. FIG. 4A) Graph showing the reaction rate based on mass spectrometry analysis. The reaction is effectively complete within 165 min. FIG. 4B) Electron density map and atomic structure of the crystalized Cys-meditope/Cys-meFab complex indicating the presence of a disulfide bond.

FIG. 5. Differential scanning fluorimetry (DSF) revealed an increase in the thermal melting point when Cys-meditopes and Cys-meFabs were allowed to interact. The graph shows the melting point for Cys-meFab alone and in combination with the Cys-meditope. The increase in melting point when the Cys-meFab and Cys-meditope were allowed to react suggests formation of a disulfide bond.

FIG. 6. Mass spectrometry indicating successful synthesis of AlexaFluor647 (AF647) conjugated to Cys-meditopes with thiopyridine (SEQ ID NO:1).

FIG. 7. AF647 thiopyridine-meditope/trastuzumab Cys-meFabs complexes bind to SKBR3 cells.

FIG. 8. Quantification of antibody binding to cells indicates that meditope-enablement and templated disulfide binding do not affect antigen binding.

FIG. 9A-9E. pH does not affect the formation of a disulfide bridge between Cys-meditopes and trastuzumab Cys-meFabs. Mass spectrometry data indicates presence of disulfide bridged Cys-meditope/Cys-meFab complexes regardless of the pH at which the reaction occurred. FIG. 9A) pH 6.5. FIG. 9B) pH 7.0. FIG. 9C) pH 7.5. FIG. 9D) pH 8.0. FIG. 9E) pH 8.5.

FIG. 10. Schematic showing how the templated Fab-meditope technology can be expanded to create heterodimeric antibodies and/or compositions of Fab fragments, biologics, and therapeutics. The creation of DBCO and azide meditopes can facilitate these technological expansions (SEQ ID NO:1).

FIG. 11A-11B. DBCO conjugated thiopyridine-meditopes form a disulfide bond with Cys-meFabs. FIG. 11A) Mass spectrometry results indicates successful synthesis of DBCO conjugated thiopyridine-meditopes (SEQ ID NO:1). FIG. 11B) Mass spectrometry data indicates DBCO conjugated thiopyridine-meditopes form disulfide bridges with Cys-meFabs.

FIG. 12A-12B. The azide conjugated thiopyridine-meditope forms a disulfide bond with the Cys-meFab. FIG. 12A) Mass spectrometry results indicate successful synthesis of azide conjugated thiopyridine-meditopes (SEQ ID NO:1). FIG. 12B) Mass spectrometry data indicates azide conjugated thiopyridine-meditopes form disulfide bridges with Cys-meFab.

FIG. 13A-13B. The azide-conjugated thiopyridine-meditope forms a disulfide bond with the meditope-enabled antibody CA19.9 mutated to include Cys at positon 175. FIG. 13A) Mass spectrometry data indicates azide-conjugated thiopyridine-meditopes (SEQ ID NO:1) form disulfide bridges with the Cys-meditope-enabled antibody CA19.9. FIG. 13B) Expanded view of the mass spectrometry data showing the peak at the expected mass.

FIG. 14A-14C. Substitution of cysteine at various meFab heavy and light chain positions on the back-side of the meFab does not affect Her2 affinity. FIG. 14A) SPR sensogram and binding affinity of the 183E meFAb. FIG. 14B) SPR sensogram and binding affinity of K208C meFab. FIG. 14C) SPR sensogram and binding affinity of T158C meFab.

FIG. 15. Mass spectrometry and chemical structure of the Arg8 octylthiol meditope (SEQ ID NO:2).

FIG. 16. Mass spectrometry reveals T158C Cys-meFabs readily form covalent linkage with Cys-meditopes through a disulfide bridge.

FIG. 17. Crystal structure of a trastuzumab meFab. Left panel shows a front view of the Cys-meFab including a thiopyridine-meditope. LC and HC denote light chain and heavy chain portions of the meFab, respectively. Right panel shows 900 rotation of the meFab. This view shows the extension of the thiopyridine-meditope through the Fab hole.

FIG. 18. Cross-sectional schematic of an azide conjugated thiopyridine-meditope interacting with a meFab. The azide conjugated thiopyridine-meditope can be further conjugated to high affinity peptides or small molecules.

FIG. 19. Cysteine can be substituted at positon 143 of the meFab light chain (LC) to guide disulfide formation with a Cys-meditope.

FIG. 20A-20B. Meditopes can be used to direct disulfide linkage to the light change. FIG. 20A) Depiction of proximity of the Cys-meditope to a light chain (LC) 102 cysteine (C). FIG. 20B) Depiction of proximity of the Cys-meditope to a light chain (LC) 142 cysteine (C).

FIG. 21. Mass spectrometry analysis of meditope including a thiopyridine at Cys13.

FIG. 22A-22B. The Cys6 thiopyridine-meditope forms a disulfide bond with 175C of the meditope-enabled antibody. FIG. 22A) Mass spectrometry data indicates formation of disulfide bridges. FIG. 22B) Expanded view of the mass spectrometry data showing the peak at the expected mass.

FIG. 23. The position of the meditope tag on anti-CD16 nanobody affects ADCC activity. An ADCC assay was performed with SKBR-3 cells using the following covalent complexes or control antibodies: cys-memAb (IgG1) (control antigen binding domain including a non-CDR peptide binding region as provided herein); CD16-Fab C-term. complex (a covalent complex provided herein, wherein the antigen binding domain is a meditope-enabled trastuzumab domain (a meditope-enabled antigen binding domain including the trastuzumab paratope) including a non-CDR peptide binding region as provided herein and wherein R²⁰ of the peptide compound is a CD16 nanobody moiety); CD16-Fab N-term. complex (a covalent complex provided herein, wherein the antigen binding domain is a meditope-enabled trastuzumab domain including a non-CDR peptide binding region as provided herein and wherein R¹⁰ of the peptide compound is a CD16 nanobody moiety); cys-Fab (a meditope-enabled trastuzumab domain including a non-CDR peptide binding region as provided herein) and CD16 (a CD16 nanobody).

FIGS. 24A-24E. Biophysical characterization of disulfide formation using the cysteine meditope. FIG. 24A. A schematic of the meditope-antibody templated disulfide formation: Cysteine residues were introduced at the interface of the meditope and the heavy chain of meTras 183E to drive the formation of a disulfide bond. FIG. 24B. Diffraction data indicates the formation of disulfide bond between the 175Cys heavy chain and the SQFDA(Ph)₂CTRRLQSGGSK meditope. The light chain is shown. FIG. 24C. LC/MS was used to follow the formation of the disulfide bond formation between 175Cys Fab and SQFDA(Ph)₂CTRRLQSGGSK. Top panel highlighting the initial sample, a midpoint (60 mins) and the fully reacted sample (120 mins) with mass shift corresponding to a single meditope (1846 Da). Bottom panel shows the reaction rate at different time points. FIG. 24D. Differential scanning fluorimetry indicates a large increased thermal denaturation temperature (Tm) of the 175C Fab after disulfide conjugation. The hash mark indicates the inflection point (e.g., Tm) for each construct. FIG. 24E. Surface plasmon resonance using 183E, 175Cys, and 175Cys Trastuzumab Fab conjugated with SQFDA(Ph)₂CTRRLQSGGSK as ligands with the extracellular domain of HER2 as the analyte fixed to the SPR chip indicates that the antigen affinity is indistinguishable from the parental Fab despite the introduction of cysteine 175 or the disulfide bond to the cys-meditope.

FIGS. 25A-25F. Functionalization using bio-orthogonal chemical groups. FIG. 25A. DBCO-Polyethylenegycol polymer (PEG-30k), added to 175Cys Fab-SQFDA(Ph)₂CTRRLQSGGSK-azido meditope through copper free click chemistry, produced a substantial shift under non-reducing conditions on an SDS-PAGE gel. FIG. 25B. 175Cys variants of meditope-enabled trastuzumab Fab and three distinct αCD3 Fabs were reacted with SQFDA(Ph)₂CTRRLQSGGSK bearing tetrazine or TCO, respectively, and mixed at 1:1 ratio. Each combination produced a substantial shift under non-reducing conditions on an SDS-PAGE gel, indicating the formation of a BiTE. Under strong reducing conditions, the components migrated at the expected mass. FIG. 25C. 175Cys trastuzumab IgGs conjugated with DM1- or MMAE-cysteine meditopes at near stoichiometric concentrations (e.g., 2.2 meditope-toxins to 1 IgG). Lysis of SKBR3, a HER2 positive tumor cell line, by the cys-meditope, trastuzumab ADCs were quantified. The EC50 is comparable to clinical trastuzumab DM1. Tumor cell lysis by clinical trastuzumab (e.g., naked antibody) is much less effective. FIG. 25D. Analytic cytometry shows 175Cys IgG binds SKBR3 cells. Clinical trastuzumab, A175C IgG, and A175C IgG carrying SQFDA(Ph)₂CTRRLQSGGSK-Alexa647 meditope were incubated with SKBR3 cells, stained with anti-human IgG Fc secondary antibody conjugated to Alexa488, and analyzed by FACs. Cells without treatment are shown. FIG. 25E. SKBR3 cells were stained with trastuzumab 175Cys IgG conjugated to SQFDA(Ph)₂CTRRLQSGGSK-Alexa647 meditope. FIG. 25F. MCF-7 tumor bearing mice were imaged with trastuzumab 175Cys IgG conjugated with SQFDA(Ph)₂CTRRLQSGGSK-Alexa647 meditope. The mouse on the left has a tumor in the shoulder, and the mouse on the right has a tumor in the lower flank. Tumors were removed and imaged ex vivo after 24 hours.

FIGS. 26A-26I. Functionalization of Fabs using genetically encoded biologics. FIG. 26A. Schematic of moxGFP bearing the cysteine meditope sequence at the N-, C- or both (NC) termini. The moxGFP with meditope and the meditope at each termini. FIG. 26B. Conjugation of the meditope-GFP variants to 175Cys trastuzumab Fab produced the expected shift in mass under non-reducing condition using SDS-PAGE. FIG. 26C. SPR studies of the N-, C- and NC-/Fab complexes indicates that the conjugated GFP variants do not reduce antigen binding. Representative traces for each complex at 313 picomolar trace are shown. The slightly longer off rate from the NC-GFP/Fab likely indicates multivalent binding. Complete titrations are shown for each construct in FIGS. 33A-33D. FIG. 26D. Schematic of N-terminal and C-terminal meditope αCD16 nanobody conjugated to 175Cys trastuzumab Fab. The light chain and heavy chains are shown. The αCD16 domain is drawn and the CDRs are shown. The genetically fused meditope is shown. FIG. 26E. SDS-PAGE of 175Cys trastuzumab Fab with αCD16 containing an N- or C-terminal meditope tag produces the expected shift in mass under non-reducing conditions. Reduction of the complexes shows the complexes dissociates into their individual components. FIG. 26F. In vitro ADCC assay using bispecifics from FIG. 26D and IgG1 (indicated on the legend). The N-terminal αCD16/Fab potently activates ADCC pathway with SKBR3 cells as the target and Jurkat cells expressing FcγRIIIa and luciferase controlled by NFAT activation as the effector. FIG. 26G. Schematic of ZHER2 with meditope tag prior to reaction with 175Cys αCD3 Fab. As before, the light and heavy chains are shown. The meditope tag is shown and ZHER2 is shown. FIG. 26H. SDS-PAGE of the three 175Cys αCD3 Fabs with ZHER2 containing an N-terminal meditope tag also produces the expected shift under non-reducing conditions. FIG. 26I. In vitro Jurkat activation by mPACT'ed BiTEs from FIG. 26G using shows substantial activation whereas Fabs only do not.

FIG. 27. Maps of the meditope site for the A175C Tras Fabs. Shown are the maps for three meditopes: Ac-SQFDFCTRRLQSGGSK (SEQ ID NO:27), Ac-SQFDA(Ph)₂CTRRLQSGGSK (SEQ ID NO:28), and Ac-CQFDLSTRRLKC-Am (SEQ ID NO:44).

FIG. 28. Mass-spec results showing no reaction between Fab and meditope combinations that are unable to form disulfide. The top panel shows that the serine variant, Ac-SQFDA(Ph)₂STRRLQSGGSK (SEQ ID NO:24), does not react with the Ala175Cys Fab. When the Ala175Cys cysteine is blocked with iodoacetamide, the cysteine meditope (Ac-SQFDA(Ph)₂CTRRLQSGGSK (SEQ ID NO:28) no longer reacts (middle panel). Finally, the cysteine meditope (Ac-SQFDA(Ph)₂CTRRLQSGGSK) and the parental Fab are not reactive (bottom panel).

FIG. 29. Comparison of increase in melting temperature of I83E in the presence of different meditopes. I83E Fab was mixed with different ratios of each meditope, and the melting temperature was measured by DSF.

FIG. 30. αCD3/Trastuzumab Fab click products activate Jurkat cells in the presence of SKBR3 cells. NFAT activation leads to expression of luceriferase in the Jurkat cells. The NFAT pathway is only activated by the constructs containing both αCD3 and Trastuzumab Fabs clicked together. The Fabs alone fail to illicit a response.

FIG. 31. HPLC traces of the drug conjugates used in cellular studies. The top panel shows the MMAE conjugate, and the lower panel shows the DM1 conjugate.

FIG. 32. A175C Fab reacted with the fluorescent protein, mEos 3.2, carrying an n-terminal meditope tag. mEos 3.2 contains multiple cysteine residues, aside from the cysteine in the meditope, giving rise to a ladder effect in the absence of A175C Fab. The Fab appears completely exhausted by 4 hours, indicating complete complex formation.

FIGS. 33A-33D. SPR traces for HER2 binding of moxGFP/Trastuzumab 175Cys conjugates. FIG. 33A. moxGFP alone. FIG. 33B. N-moxGFP conjugate. FIG. 33C. c-moxGFP conjugate. FIG. 33D. NC-moxGFP conjugate.

FIGS. 34A-34B. The stability of the Tras Ala175Cys-αCD16 conjugate. In FIG. 34A, the conjugate was incubated in rat serum for 14 days at 37° C., and stained for the kappa light chain on the Fab. The disulfide bond appears intact after 14 days, as no free Fab appears. In FIG. 34B, the conjugate is exposed to increasing amounts of reduced glutathione in either its native state (left) or SDS denatured state (right). The native state is far more resistant to reduction indicating the stabilization of the disulfide.

FIG. 35. Thermal stability of αCD3 Fabs before and after conjugation with ZHER2.

FIG. 36. Jurkat cell activation by ZHER2-αCD3 BiTEs with MCF7 cells.

FIG. 37. Denaturing mass-spec of the T158C after reaction with 5-diphenyl, 8-octyl thiol meditope.

FIG. 38. Crystal structure with map for T158C meditope disulfide. The meditope, the light chain, and the heavy chain are shown.

### DETAILED DESCRIPTION OF THE INVENTION

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Where substituent groups are specified by their conventional chemical formulae, written from left to right, they equally encompass the chemically identical substituents that would result from writing the structure from right to left, e.g., —CH₂O-is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R¹— represents both —C(O)₂R′—and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH2)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

A fused ring heterocycloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

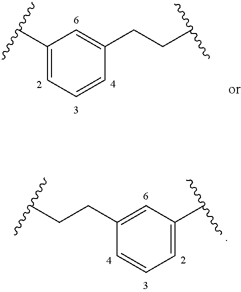 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,”, “cycloalkyl”, “heterocycloalkyl”, “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, =NR′, =N—OR′, —NR′R″, —SR′, -halogen, —SiR′R″R″, —OC(O)R′, —C(O)R′, —CO₂R′, —CONR′R″, —OC(O)NR′R″, —NR″C(O)R′, —NR′—C(O)NR″R″, —NR″C(O)₂R′, —NR—C(NR′R″R″′)=NR″ ″, —NR—C(NR′R″)=NR′″, —S(O)R′, —S(O)₂R′, —S(O)₂NR′R″, —NRSO₂R′, —NR′NR″R″, —ONR′R″, —NR′C(O)NR″NR″′R″ ″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R″′, and R″ ″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R″′, and R″ ″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R″, —OC(O)R′, —C(O)R′, —CO₂R′, —CONR′R″, —OC(O)NR′R″, —NR″C(O)R′, —NR′—C(O)NR″R″, —NR″C(O)₂R′, —NR—C(NR′R″R″′)=NR″ ″, —NR—C(NR′R″)=NR′″, —S(O)R′, —S(O)₂R′, —S(O)₂NR′R″, —NRSO₂R′, —NR′NR″R″, —ONR′R″, —NR′C(O)NR″NR″′R″ ″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R″′, and R″ ″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R″′, and R″ ″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted In some embodiments, each substituted group rings means that at least one ring is substituted and each substituent may optionally be different.

Where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13c}, R^{13D}, etc., wherein each of R^{13A}, R^{13B}, R^{13C}, R^{13D}, etc. is defined within the scope of the definition of R¹³ and optionally differently

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R″′)_{d}—, where s and d are independently integers of from 0 to 3, and X′ is —O—, —NR′—, —S—, —S(O)—, —S(O)₂—, or —S(O)₂NR′—. The substituents R, R′, R″, and R″′ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties: (A) oxo, halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and

(B) alkyl, heteroalkyl, cycloalkyl, heterocycloalkyl, aryl, heteroaryl, substituted with at least one substituent selected from:
(i) oxo, halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(ii) alkyl, heteroalkyl, cycloalkyl, heterocycloalkyl, aryl, heteroaryl, substituted with at least one substituent selected from:
   (a) oxo, halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, unsubstituted alkyl (e.g., C₁-C₈ alkyl, C₁-C₆alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and

(b) alkyl, heteroalkyl, cycloalkyl, heterocycloalkyl, aryl, heteroaryl, substituted with at least one substituent selected from: oxo, halogen, —CCl₃, —CBr₃, —CF₃, —Cl₃, —CN, —OH, —NH₂, —COOH, —CONH₂, —N₀₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

The term “oxo,” as used herein, means an oxygen that is double bonded to a carbon atom.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene. In some embodiments, the compound is a chemical species set forth in the Examples section, figures, or tables below.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted peptidyl moiety, substituted or unsubstituted peptide sequence, substituted or unsubstituted amino acid, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted peptidyl moiety, unsubstituted peptide sequence, unsubstituted amino acid, substituted or unsubstituted amino acid, is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted peptidyl moiety, substituted or unsubstituted peptide sequence, substituted or unsubstituted amino acid, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted peptidyl moiety, substituted peptide sequence, substituted amino acid, substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted peptidyl moiety, substituted peptide sequence, substituted amino acid, substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., a substituted peptidyl moiety, substituted peptide sequence, substituted amino acid, substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., a substituted peptidyl moiety, substituted peptide sequence, substituted amino acid, substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., a substituted peptidyl moiety, substituted peptide sequence, substituted amino acid, substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Where a moiety is substituted (e.g., a substituted peptidyl moiety, substituted peptide sequence, substituted amino acid, substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively), the moiety is substituted with at least one substituent (e.g., a substituent group, a size-limited substituent group, or lower substituent group) and each substituent is optionally different. Additionally, where multiple substituents are present on a moiety, each substituent may be optionally differently.

Certain compounds of the present disclosure possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center.

Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

As used herein, the term “conjugate” refers to the association between atoms or molecules. The association can be direct or indirect. For example, a conjugate between an antigen binding domain and a peptide compound can be direct, e.g., by covalent bond (e.g., a disulfide bond), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, conjugates are formed using conjugate chemistry including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

Useful reactive moieties or functional groups used for conjugate chemistries (including “click chemistries” as known in the art) herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold;
(h) amine or sulfhydryl groups, which can be, for example, acylated, alkylated or oxidized; (i) alkenes, which can undergo, for example, cycloadditions, acylation, Michael addition, etc.;
(j) epoxides, which can react with, for example, amines and hydroxyl compounds;
(k) phosphoramidites and other standard functional groups useful in nucleic acid synthesis;
(l) metal silicon oxide bonding;
(m) metal bonding to reactive phosphorus groups (e.g. phosphines) to form, for example, phosphate diester bonds; and
(n) sulfones, for example, vinyl sulfone.

Chemical synthesis of compositions by joining small modular units using conjugate (“click”) chemistry is well known in the art and described, for example, in H. C. Kolb, M. G. Finn and K. B. Sharpless ((2001). “Click Chemistry: Diverse Chemical Function from a Few Good Reactions”. Angewandte Chemie International Edition 40 (11): 2004-2021); R. A. Evans ((2007). “The Rise of Azide-Alkyne 1,3-Dipolar ‘Click’Cycloaddition and its Application to Polymer Science and Surface Modification”. Australian Journal of Chemistry 60 (6): 384-395; W. C. Guida et al. Med. Res. Rev. p 3 1996; Spiteri, Christian and Moses, John E. ((2010). “Copper-Catalyzed Azide-Alkyne Cycloaddition: Regioselective Synthesis of 1,4,5-Trisubstituted 1,2,3-Triazoles”. Angewandte Chemie International Edition 49 (1): 31-33); Hoyle, Charles E. and Bowman, Christopher N. ((2010). “Thiol-Ene Click Chemistry”. Angewandte Chemie International Edition 49 (9): 1540-1573); Blackman, Melissa L. and Royzen, Maksim and Fox, Joseph M. ((2008). “Tetrazine Ligation: Fast Bioconjugation Based on Inverse-Electron-Demand Diels-Alder Reactivity”. Journal of the American Chemical Society 130 (41): 13518-13519); Devaraj, Neal K. and Weissleder, Ralph and Hilderbrand, Scott A. ((2008). “Tetrazine Based Cycloadditions: Application to Pretargeted Live Cell Labeling”. Bioconjugate Chemistry 19 (12): 2297-2299); Stöckmann, Henning; Neves, Andre; Stairs, Shaun; Brindle, Kevin; Leeper, Finian ((2011). “Exploring isonitrile-based click chemistry for ligation with biomolecules”. Organic & Biomolecular Chemistry), all of which are hereby incorporated by reference in their entirety and for all purposes.

The reactive functional groups and reactive moieties can be chosen such that they do not participate in, or interfere with, the chemical stability of the antigen binding domain and the peptide compound described herein.

The term “reactive moiety” as provided herein refers to a chemically functional group of a molecule (e.g., compound or antigen binding domain provided herein), which is capable of forming a covalent or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like) (e.g., covalent or non-covalent bonds) with another reactive moiety of the same or a different molecule. In embodiments, the reactive moiety is a click chemistry reactive group or click chemistry reactive moiety (i.e., a reactive moiety or functional group useable for conjugate chemistries (including “click chemistries” as known in the art)). As described above a click chemistry reactive group is a chemically functional group useful for conjugate chemistry. Thus, in embodiments, the reactive moiety is an azide moiety. In embodiments, the reactive moiety has the structure of —N═N⁺═N⁻. In embodiments, the reactive moiety is alkyne.

In embodiments, the reactive moiety is DBCO. The term “DBCO” as provided herein refers in a customary sense to dibenzocyclooctyl identified by PubChem No. 77078258 or any reactive group including DBCO. In embodiments, the reactive moiety has or includes the structure:

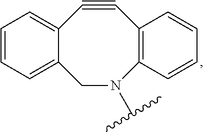 [SMILES: *N1Cc2ccccc2C#Cc2ccccc21]

wherein [symbol] indicates the point of attament to the reminder of the molecule. In embodiments, the reactive moiety is 30 kDa pegylated-DBCO.

In embodiments, the reactive moiety is a trans-cyclooctene (TCO) moiety. The term “TCO” as provided herein refers in a customary sense to trans-cyclooctene identified by PubChem No. 89994470 or any reactive group including TCO. In embodiments, the reactive moiety has or includes the structure:

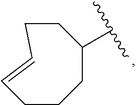 [SMILES: *C1CC/C=C/CCC1]

wherein [symbol] indicates the point of attament to the reminder of the molecule.

In embodiments, the reactive moiety is a tetrazine moiety. The term “tetrazine” as provided herein refers in a customary sense to tetrazine identified by PubChem No. 9263 or any reactive group including tetrazine. In embodiments, the reactive moiety has or includes the structure:

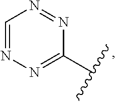 [SMILES: *c1nncnn1]

wherein [symbol] indicates the point of attament to the reminder of the molecule.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

The symbol “ [symbol] ” denotes the point of attachment of a chemical moiety to the remainder of a molecule or chemical formula.

Descriptions of compounds (peptide compounds) of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

A “therapeutic agent” or “therapeutic moiety” as used herein refers to an agent (e.g., compound or composition) that when administered to a subject will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

The term “peptidyl” and “peptidyl moiety” refers to a peptide attached to the remainder of the molecule (e.g. R¹, R², or -L^{3A}-L^{3B}-R³ of the peptide compound of formula (I), (IA), (IB), (II) or (IIA)). A peptidyl moiety may be substituted with a chemical linker that serves to attach the peptidyl moiety to R¹, R², or -L^{3A}-L^{3B}-R³ of the peptide compound of formula (I) or formula (II). The peptidyl moiety may also be substituted with additional chemical moieties (e.g., additional R substituents). In embodiments, the peptidyl moiety forms part of the peptide compound of formula (I). In embodiments, the peptidyl moiety forms part of the peptide compound of formula (II). The term “meditope” as used herein refers to a peptidyl moiety included in the peptide compound as described herein. Thus, in embodiments, a meditope is a peptidyl moiety.

The peptidyl moiety (e.g., meditope) may be a linear or a cyclic peptide moiety. Various methods for cyclization of a peptide moiety may be used, e.g., to address in vivo stability and to enable chemoselective control for subsequent conjugation chemistry. In some embodiments, the cyclization strategy is a lactam cyclization strategy, including head-to-tail (head-tail) lactam cyclization (between the terminal residues of the acyclic peptide) and/or lactam linkage between other residues. Lactam formation may also be affected by incorporating residues such as glycine, β-Ala, and/or 7-aminoheptanoic acid, and the like, into the acyclic peptide cyclization precursors to produce different lactam ring sizes and modes of connectivity. Additional cyclization strategies such as “click” chemistry and olefin metathesis also can be used. Such methods of peptide and peptidomimetic cyclization are well known in the art. In embodiments, the peptidyl moiety (e.g., meditope) is a linear peptidyl moiety (e.g., linear meditope). In embodiments, the peptidyl moiety (e.g., meditope) is a cyclic peptidyl moiety (e.g., cyclic meditope).

The term “peptide compound” refers to a compound including a peptidyl portion. In embodiments, the peptide compound includes a peptide or peptidyl moiety directly (covalently) or indirectly (non-covalently) attached to one or more chemical substituents (e.g., R¹, R², or -L^{3A}-L^{3B}-R³). In embodiments, the peptide compound includes a peptide or peptidyl moiety covalently attached to one or more chemical substituents. In embodiments, the peptide compound includes a peptidyl moiety. In embodiments, the peptide compound is a compound of formula (I). In embodiments, the peptide compound is a compound of formula (II).

A “label,” “detectable agent,” or “detectable moiety” is a composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, 64Cu, ⁶⁷Cu, ⁶⁷Ga, ⁶⁸Ga, ⁷⁷As, ⁸⁶y, ⁹⁰y. ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹p, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴, ¹⁹⁸Au, ¹⁹⁹Au, ²¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, 64Cu, ⁶⁷Cu, ⁶⁷Ga, ⁶⁸Ga, ⁷⁷As, ⁸⁶y, ⁹⁰y. ⁸⁹Sr, ⁸⁹Zr, ⁹⁴Tc, ⁹⁴Tc, ⁹⁹mTc, ⁹⁹Mo, ¹⁰⁵Pd, 105Rh ¹¹¹Ag, ¹¹¹In, ¹²³I, ¹²⁴I, ¹²⁵I, ¹³¹I, ¹⁴²Pr, ¹⁴³Pr, ¹⁴⁹Pm ¹⁵³Sm, ¹⁵⁴⁻¹⁵⁸¹Gd, ¹⁶¹Tb, ¹⁶⁶Dy, ¹⁶⁶Ho ¹⁶⁹Er, ¹⁷⁵Lu ¹⁷⁷Lu, ¹⁸⁶Re, ¹⁸⁸Re, ¹⁸⁹Re, ¹⁹⁴I, ¹⁹⁸Au, ¹⁹⁹Au, ²¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

A “labeled protein or polypeptide” is one that is bound, either covalently, through a linker or a chemical bond, or noncovalently, through ionic, van der Waals, electrostatic, or hydrogen bonds to a label such that the presence of the labeled protein or polypeptide may be detected by detecting the presence of the label bound to the labeled protein or polypeptide. Alternatively, methods using high affinity interactions may achieve the same results where one of a pair of binding partners binds to the other, e.g., biotin, streptavidin.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. For example, a selected residue in a selected antibody (or antigen binding domain) corresponds to light chain threonine at Kabat position 40, when the selected residue occupies the same essential spatial or other structural relationship as a light chain threonine at Kabat position 40. In some embodiments, where a selected protein is aligned for maximum homology with the light chain of an antibody (or antigen binding domain), the position in the aligned selected protein aligning with threonine 40 is said to correspond to threonine 40. Instead of a primary sequence alignment, a three dimensional structural alignment can also be used, e.g., where the structure of the selected protein is aligned for maximum correspondence with the light chain threonine at Kabat position 40, and the overall structures compared. In this case, an amino acid that occupies the same essential position as threonine 40 in the structural model is said to correspond to the threonine 40 residue.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids sequences encode any given amino acid residue. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different from the general chemical structure of a nucleic acid, but that functions in a manner similar to a naturally occurring nucleic acid. Examples of such analogues include, without limitation, phosphorothiolates, phosphoramidates, methyl phosphonates, chiral-methyl phosphonates, 2-O-methyl ribonucleotides, and peptide-nucleic acids (PNAs).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length. The present invention includes polypeptides that are substantially identical to any of SEQ ID NOs:1-21.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion, the gene is positioned in a predictable manner between two transposon linker sequences that allow insertion into the host genome as well as subsequent excision. Stable expression of a transfected gene can further be accomplished by infecting a cell with a lentiviral vector, which after infection forms part of (integrates into) the cellular genome thereby resulting in stable expression of the gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetofection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The term “modulation”, “modulate”, or “modulator” are used in accordance with their plain ordinary meaning and refer to the act of changing or varying one or more properties. “Modulator” refers to a composition that increases or decreases the level of a target molecule or the function of a target molecule or the physical state of the target of the molecule. “Modulation” refers to the process of changing or varying one or more properties. For example, as applied to the effects of a modulator on a biological target, to modulate means to change by increasing or decreasing a property or function of the biological target or the amount of the biological target.

As defined herein, the term “inhibition”, “inhibit”, “inhibiting” and the like in reference to a protein-inhibitor (e.g. antagonist) interaction means negatively affecting (e.g. decreasing) the activity or function of the protein relative to the activity or function of the protein in the absence of the inhibitor. In embodiments inhibition refers to reduction of a disease or symptoms of disease. Thus, in embodiments, inhibition includes, at least in part, partially or totally blocking stimulation, decreasing, preventing, or delaying activation, or inactivating, desensitizing, or down-regulating signal transduction or enzymatic activity or the amount of a protein. The amount of inhibition may be 10%, 20%, 30%, 40%, 50%, 60%, 70%, 80%, 90%, 100% or less in comparison to a control in the absence of the antagonist. In embodiments, the inhibition is 1.5-fold, 2-fold, 3-fold, 4-fold, 5-fold, 10-fold, or more than the expression or activity in the absence of the antagonist.

As defined herein, the term “activation”, “activate”, “activating” and the like in reference to a protein-activator (e.g. agonist) interaction means positively affecting (e.g. increasing) the activity or function of the relative to the activity or function of the protein in the absence of the activator (e.g. composition described herein). Thus, in embodiments, activation may include, at least in part, partially or totally increasing stimulation, increasing or enabling activation, or activating, sensitizing, or up-regulating signal transduction or enzymatic activity or the amount of a protein decreased in a disease. The amount of activation may be 10%, 20%, 30%, 40%, 50%, 60%, 70%, 80%, 90%, 100% or more in comparison to a control in the absence of the agonist. In embodiments, the activation is 1.5-fold, 2-fold, 3-fold, 4-fold, 5-fold, 10-fold, or more than the expression or activity in the absence of the agonist.

The term “recombinant” when used with reference, for example, to a cell, a nucleic acid, a protein, or a vector, indicates that the cell, nucleic acid, protein or vector has been modified by or is the result of laboratory methods. Thus, for example, recombinant proteins include proteins produced by laboratory methods. Recombinant proteins can include amino acid residues not found within the native (non-recombinant) form of the protein or can be include amino acid residues that have been modified, e.g., labeled.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody plays a significant role in determining the specificity and affinity of binding. In some embodiments, antibodies or fragments of antibodies may be derived from different organisms, including humans, mice, rats, hamsters, camels, etc. Antibodies of the invention may include antibodies that have been modified or mutated at one or more amino acid positions to improve or modulate a desired function of the antibody (e.g. glycosylation, expression, antigen recognition, effector functions, antigen binding, specificity, etc.).

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e. fragment crystallizable region) is the “base” or “tail” of an immunoglobulin and is typically composed of two heavy chains that contribute two or three constant domains depending on the class of the antibody. By binding to specific proteins the Fc region ensures that each antibody generates an appropriate immune response for a given antigen. The Fc region also binds to various cell receptors, such as Fc receptors, and other immune molecules, such as complement proteins.

The term “antigen” as provided herein refers to molecules capable of binding to the antibody binding domain provided herein, wherein the binding site is not the peptide binding site.

Antibodies exist, for example, as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH—CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially the antigen binding portion with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of a mAb is the region of its antigen to which the mAb binds. Two antibodies bind to the same or overlapping epitope if each competitively inhibits (blocks) binding of the other to the antigen. That is, a 1x, 5x, 10x, 20x or 100x excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

An “antibody variant” as provided herein refers to a polypeptide capable of binding to an antigen and including one or more structural domains of an antibody or fragment thereof. Non-limiting examples of antibody variants include single-domain antibodies or nanobodies, affibodies (polypeptides smaller than monoclonal antibodies (e.g., about 6 kDA) and capable of binding antigens with high affinity and imitating monoclonal antibodies, monospecific Fab2, bispecific Fab2, trispecific Fab3, monovalent IgGs, scFv, bispecific diabodies, trispecific triabodies, scFv-Fc, minibodies, IgNAR, V-NAR, hcIgG, VhH, or peptibodies. A “nanobody” or “single domain antibody” as described herein is commonly well known in the art and refers to an antibody fragment consisting of a single monomeric variable antibody domain. Like a whole antibody, it is able to bind selectively to a specific antigen. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)).

Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

A “therapeutic antibody” as provided herein refers to any antibody or functional fragment thereof (e.g., a nanobody) that is used to treat cancer, autoimmune diseases, transplant rejection, cardiovascular disease or other diseases or conditions such as those described herein. Non-limiting examples of therapeutic antibodies include murine antibodies, murinized or humanized chimera antibodies or human antibodies including, but not limited to, Erbitux (cetuximab), ReoPro (abciximab), Simulect (basiliximab), Remicade (infliximab); Orthoclone OKT3 (muromonab-CD3); Rituxan (rituximab), Bexxar (tositumomab) Humira (adalimumab), Campath (alemtuzumab), Simulect (basiliximab), Avastin (bevacizumab), Cimzia (certolizumab pegol), Zenapax (daclizumab), Soliris (eculizumab), Raptiva (efalizumab), Mylotarg (gemtuzumab), Zevalin (ibritumomab tiuxetan), Tysabri (natalizumab), Xolair (omalizumab), Synagis (palivizumab), Vectibix (panitumumab), Lucentis (ranibizumab), and Herceptin (trastuzumab).

Techniques for conjugating therapeutic agents to antibodies are well known (see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2“d Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)). As used herein, the term “antibody-drug conjugate” or “ADC” refers to a therapeutic agent conjugated or otherwise covalently bound to an antibody.

The phrase “specifically (or selectively) binds to an antibody” or “specifically (or selectively) immunoreactive with,” when referring to a protein or peptide refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions typically requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity). protein).

A “ligand” refers to an agent, e.g., a polypeptide or other molecule, capable of binding to a receptor.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a peptide compound as described herein and an antigen binding domain. In embodiments, contacting includes, for example, allowing a compound described herein to interact with an antigen binding domain resulting in covalent linkage (e.g., through a disulfide bond).

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with a compound, pharmaceutical composition, or method provided herein. In embodiments, the disease is cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma).

The terms “treating”, or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being.

The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease. In embodiments, “treating” refers to treatment of cancer.

An “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “therapeutically effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like, that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

Pharmaceutical compositions may include compositions wherein the active ingredient (e.g. compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule, and/or reducing, eliminating, or slowing the progression of disease symptoms.

### Covalent Complexes

Provided herein are, inter alia, compositions including monoclonal antibodies (mAbs) and antibody fragments that are covalently bound through a disulfide linkage to a peptide compound (e.g., a compound of formula (I), (IA), (IB), (II) or (IIA)). The disulfide linkage is formed between a cysteine residue of the antibody or Fab and a peptide compound amino acid having a side chain that includes a thiol moiety (i.e., a thiol side chain amino acid). The cysteine residue may be part of a the heavy chain variable (VH) region, the light chain variable (VL) region, the heavy chain constant (CH1) region or the light chain constant (CL) of the antibody or Fab. The thiol side chain amino acid may be a cysteine, a protected cysteine (a cysteine covalently attached to a protecting group) or an arginine substituted with a thiol-substituent (an octyl-thiol-substituted arginine). Surprisingly, Applicants have found that the covalent attachment of the peptide compound (e.g., peptide compound of formula (I), (IA), (IB), (II) or (IIA)) to the antibody or fragment thereof improves the thermal stability and therapeutic properties of the antibody or Fab. Further, Applicants have found that the antibody or Fab provided herein binds to an antigen with increased affinity when bound through a disulfide linkage to the peptide compound. Thus, the functionalized antibodies provided herein are endowed with the ability to better target and affect a site or cell. In addition, a large variety of diagnostic, therapeutic, and detectable agents may be conjugated to the peptide compound provided herein including embodiments thereof, thereby making the covalent complexes provided herein useful agents for a variety of therapeutic and diagnostic purposes.

In a first aspect, there is provided a covalent complex including: (i) an antigen binding domain including: (1) a central hole enclosed by the heavy chain variable (VH) region, the light chain variable (VL) region, the heavy chain constant (CH1) region and the light chain constant (CL) region of the antigen binding domain between a first cavity and a second cavity; and (2) a non-CDR peptide binding region including: (a) the first cavity lined by a first set of amino acid residues of the VH, VL, CH1, and CL regions of the antigen binding domain; (b) the second cavity lined by a second set of amino acid residues of the VH, VL, CH1, and CL regions of the antigen binding domain; or (c) a hole region enclosing the hole between the first cavity and the second cavity, the hole region lined by a third set of amino acid residues of the VH, VL, CH1, and CL regions of the antigen binding domain; wherein the non-CDR peptide binding region includes a first cysteine; and (ii) a peptide compound including a thiol side chain amino acid covalently bound to the antigen binding domain through a disulfide linkage between the first cysteine and the thiol side chain amino acid.

The “heavy chain variable (VH) region” as provided herein is a domain which includes the variable region of a heavy chain of an antibody or a fragment thereof. Likewise, the “light chain variable (VL) region” as provided herein is a domain including the variable region of a light chain of an antibody or a fragment thereof. In embodiments, the heavy chain variable (VH) region is the variable region of the heavy chain of an antibody. In embodiments, the heavy chain variable (VH) region is the variable region of the heavy chain of an antibody fragment. In embodiments, the heavy chain variable (VH) region is the variable region of the heavy chain of a Fab. In embodiments, the light chain variable (VL) region is the variable region of the light chain of an antibody. In embodiments, the light chain variable (VL) region is the variable region of the light chain of an antibody fragment. In embodiments, the light chain variable (VL) region is the variable region of the light chain of a Fab.

An “antigen binding domain” as provided herein is a region of an antibody that binds to an antigen (epitope). As described above, the antigen binding domain is generally composed of one constant and one variable domain of each of the heavy and the light chain (VL, VH, CL and CH1, respectively). The paratope or antigen-binding site is formed on the N-terminus of the antigen binding domain. The two variable domains of an antigen binding domain typically bind the epitope on an antigen. In embodiments, the antigen binding domain forms part of an antibody. In embodiments, the antigen binding domain forms part of a therapeutic antibody. In embodiments, the antigen binding domain forms part of a Fab. In embodiments, the antigen binding domain is a Fab.

In embodiments, the antigen binding domain includes a heavy chain constant region (CH) and a light chain constant region (CL). In embodiments, the heavy chain constant region (CH) is the constant region of the heavy chain of an antibody or fragment thereof. In embodiments, the light chain constant region (CL) is the constant region of the light chain of an antibody or fragment thereof. In embodiments, the heavy chain constant region (CH) is the constant region of a Fab. In embodiments, the light chain constant region (CL) is the constant region of the light chain of a Fab.

In embodiments, the heavy chain constant region (CH) is the constant region of a F(ab)′2 dimer. In embodiments, the light chain constant region (CL) is the constant region of the light chain of a F(ab)′2 dimer. In embodiments, the antigen binding domain includes an Fc domain. In embodiments, the antigen binding domain is a humanized antigen binding domain. In embodiments, the antigen binding domain is a humanized mouse antigen binding domain.

In embodiments, the antigen binding domain is a meditope-enabled trastuzumab domain, a meditope-enabled pertuzumab domain, a meditope-enabled M5A domain or a meditope-enabled rituximab domain. In embodiments, the antigen binding domain is a humanized meditope-enabled rituximab domain.

In embodiments, the antigen binding domain provided herein including embodiments thereof competes for antigen binding with, specifically binds to the same antigen or epitope as, and/or contains one, more, or all CDRs (or CDRs comprising at least at or about 75, 80, 85, 90, 91, 92, 93, 94, 95, 96, 97, 98, or 99% identity to the CDRs), e.g., including a heavy chain CDR 1, 2, and/or 3 and/or a light chain CDR1, 2, and/or 3, of one or more known antibodies, including any commercially available antibody, such as abagovomab, abciximab, adalimumab, adecatumumab, alemtuzumab, altumomab, altumomab pentetate, anatumomab, anatumomab mafenatox, arcitumomab, atlizumab, basiliximab, bectumomab, ectumomab, belimumab, benralizumab, bevacizumab, brentuximab, canakinumab, capromab, capromab pendetide, catumaxomab, certolizumab, clivatuzumab tetraxetan, daclizumab, denosumab, eculizumab, edrecolomab, efalizumab, etaracizumab, ertumaxomab, fanolesomab, Fbta05, fontolizumab, gemtuzumab, girentuximab, golimumab, ibritumomab, igovomab, infliximab, ipilimumab, labetuzumab, mepolizumab, muromonab, muromonab-CD3, natalizumab, necitumumab, nimotuzumab, ofatumumab, omalizumab, oregovomab, palivizumab, panitumumab, ranibizumab, rituximab, satumomab, sulesomab, ibritumomab, ibritumomab tiuxetan, tocilizumab, tositumomab, trastuzumab, Trbs07, ustekinumab, visilizumab, votumumab, zalutumumab, and/or brodalumab; and/or anrukinzumab, bapineuzumab, dalotuzumab, demcizumab, ganitumab, inotuzumab, mavrilimumab, moxetumomab pasudotox, rilotumumab, sifalimumab, tanezumab, tralokinumab, tremelimumab, urelumab, the antibody produced by the hybridoma 10B5 (see Edelson & Unanue, Curr Opin Immunol, 2000 August; 12(4):425-31), B6H12.2 (abeam) or other anti-CD47 antibody (see Chao et al., Cell, 142, 699-713, Sep. 3, 2010).

In embodiments, the antigen binding domain specifically binds to an antigen selected from the group consisting of: CD16, CA-125, glycoprotein (GP) IIb/IIIa receptor, TNF-alpha, CD52, TAG-72, Carcinoembryonic antigen (CEA), interleukin-6 receptor (IL-6R), IL-2, interleukin-2 receptor a-chain (CD25), CD22, B-cell activating factor, interleukin-5 receptor (CD125), VEGF, VEGF-A, CD30, IL-1beta, prostate specific membrane antigen (PSMA), CD3, EpCAM, EGF receptor (EGFR), MUC1, human interleukin-2 receptor, Tac, RANK ligand, a complement protein, e.g., C5, EpCAM, CD11a, e.g., human CD11a, an integrin, e.g., alpha-v beta-3 integrin, vitronectin receptor alpha v beta 3 integrin, HER2, neu, CD3, CD15, CD20 (small and/or large loops), Interferon gamma, CD33, CA-IX, TNF alpha, CTLA-4, carcinoembryonic antigen, IL-5, CD3 epsilon, CAM, Alpha-4-integrin, IgE, e.g., IgE Fc region, an RSV antigen, e.g., F protein of respiratory syncytial virus (RSV), TAG-72, NCA-90 (granulocyte cell antigen), IL-6, GD2, GD3, IL-12, IL-23, IL-17, CTAA16.88, IL13, interleukin-1 beta, beta-amyloid, IGF-1 receptor (IGF-1R), delta-like ligand 4 (DLL4), alpha subunit of granulocyte macrophage colony stimulating factor receptor, hepatocyte growth factor, IFN-alpha, nerve growth factor, IL-13, CD326, Programmed cell death 1 ligand 1 (PD-L1, a.k.a. CD274, B7-H1), CD47, and CD137.

In embodiments, the antigen binding domain is an anti-CD16, anti-HER2, anti-CD19 protein, anti-CD20 protein, anti-CD22 protein, anti-CD30 protein, anti-CD33 protein, anti-CD44v6/7/8 protein, anti-CD123 protein, anti-CEA protein, anti-EGP-2 protein, anti-EGP-40 protein, anti-erb-B2 protein, anti-erb-B2,3,4 protein, anti-FBP protein, anti-fetal acetylcholine receptor protein, anti-GD2 protein, anti-GD3 protein, anti-Her2/neu protein, anti-IL-13R-a2 protein, anti-KDR protein, anti k-light chain protein, anti-LeY protein, anti-L1 cell adhesion molecule protein, anti-MAGE-A1 protein, anti-mesothelin protein, anti-murine CMV infected cell protein, anti-MUC2 protein, anti-NKGD2 protein, anti, oncofetal antigen protein, anti-PCSA protein, anti-PSMA protein, anti-TAA (targeted by mAb IfE) protein, anti-EGFR protein, anti-TAG-72 protein or anti-VEGF-72 protein. In embodiments, the antigen binding domain is not cetuximab.

In embodiments, the antigen binding domain includes the sequence of SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20 or SEQ ID NO:21. In embodiments, the antigen binding domain includes the sequence of SEQ ID NO:5. In embodiments, the antigen binding domain includes the sequence of SEQ ID NO:6. In embodiments, the antigen binding domain includes the sequence of SEQ ID NO:7. In embodiments, the antigen binding domain includes the sequence of SEQ ID NO:8. In embodiments, the antigen binding domain includes the sequence of SEQ ID NO:9. In embodiments, the antigen binding domain includes the sequence of SEQ ID NO:10. In embodiments, the antigen binding domain includes the sequence of SEQ ID NO:11. In embodiments, the antigen binding domain includes the sequence of SEQ ID NO:12. In embodiments, the antigen binding domain includes the sequence of SEQ ID NO:13. In embodiments, the antigen binding domain includes the sequence of SEQ ID NO:14. In embodiments, the antigen binding domain includes the sequence of SEQ ID NO:15. In embodiments, the antigen binding domain includes the sequence of SEQ ID NO:16. In embodiments, the antigen binding domain includes the sequence of SEQ ID NO:17. In embodiments, the antigen binding domain includes the sequence of SEQ ID NO:18. In embodiments, the antigen binding domain includes the sequence of SEQ ID NO:19. In embodiments, the antigen binding domain includes the sequence of SEQ ID NO:20. In embodiments, the antigen binding domain includes the sequence of SEQ ID NO:21.

The “central hole” as provided herein refers, with respect to the three-dimensional structure of an antigen binding domain (e.g., Fab), to a hole within the antigen binding domain (e.g., Fab) and is located between a first and a second cavity. The central hole as well as the first and second cavity of the antigen binding domain is lined by portions of the heavy and light chain variable and constant regions. The central hole, the first cavity and the second cavity are thus lined by amino acid residues of the VH, VL, CH1, and CL regions, respectively. The amino acid residues of the VH, VL, CH1, and CL region enclosing the central hole form a hole region. The amino acid residues lining the first cavity are referred to herein as “first set of amino acid residues.” The amino acid residues lining the second cavity are referred to herein as “second set of amino acid residues.” And the amino acid residues lining the hole region are referred to herein as “third set of amino acid residues.” The amino acid residues included in the first, second and third set of amino acid residues (i.e., the amino acid residues of the first cavity, the second cavity and the hole region) are amino acid residues of the VH, VL, CH1, and CL regions and do not form part of the CDRs. The amino acid residues included in the first cavity, the second cavity or the hole region are capable of forming a disulfide linkage with the peptide compound provided herein including embodiments thereof. Thus, the first cavity, the second cavity and the hole region provided herein form part of a non-CDR peptide binding region. The “non-CDR peptide binding region” is a region of the antigen binding domain, which is capable of binding to the peptide compound provided herein including embodiments thereof. The non-CDR peptide binding region provided herein is a region within the antigen binding domain that does not include CDR residues of the heavy chains and CDR residues of the light chains. In embodiments, the non-CDR peptide binding region includes FR residues of the heavy chains and FR residues of the light chains. In embodiments, the non-CDR peptide binding region includes framework region amino acid residues. The first cavity of the non-CDR peptide binding region provided herein may also be referred to as a “meditope binding site.” In embodiments, the meditope binding site includes the hole region.

In embodiments, amino acids of the first set of amino acid residues (amino acid residues of the first cavity) interact with the peptide compound provided herein including embodiments thereof (e.g., peptide compound of formula (I), (IA), (IB), (II) or (IIA)). In embodiments, amino acids of the second set of amino acid residues (amino acid residues of the second cavity) interact with the peptide compound provided herein including embodiments thereof (e.g., peptide compound of formula (I), (IA), (IB), (II) or (IIA)). In embodiments, amino acids of the third set of amino acid residues (amino acid residues of the hole region) interact with the peptide compound provided herein including embodiments thereof (e.g., peptide compound of formula (I), (IA), (IB), (II) or (IIA)). In embodiments, amino acids of the first and the second set of amino acid residues (amino acid residues of the first and the second cavity) interact with the peptide compound provided herein including embodiments thereof (e.g., peptide compound of formula (I), (IA), (IB), (II) or (IIA)). In embodiments, amino acids of the first and the third set of amino acid residues (amino acid residues of the first cavity and the hole region) interact with the peptide compound provided herein including embodiments thereof (e.g., peptide compound of formula (I), (IA), (IB), (II) or (IIA)). In embodiments, amino acids of the second and the third set of amino acid residues (amino acid residues of the second cavity and the hole region) interact with the peptide compound provided herein including embodiments thereof (e.g., peptide compound of formula (I), (IA), (IB), (II) or (IIA)). In embodiments, amino acids of the first, second and the third set of amino acid residues (amino acid residues of the first and second cavity and the hole region) interact with the peptide compound provided herein including embodiments thereof (e.g., peptide compound of formula (I), (IA), (IB), (II) or (IIA)).

In embodiments, the peptide compound that binds to the non-CDR peptide binding region does not impact (e.g. measurably impact) the binding of the antigen binding domain to the epitope. In other words, in embodiments, occupancy of this site does not affect antigen binding. In embodiments, the non-CDR peptide binding region interacts with the peptidyl moiety (e.g., a meditope) of the peptide compound provided herein including embodiments thereof (e.g., a compound of formula (I), (IA), (IB), (II) or (IIA)). The amino acid residues capable of interacting with the peptide compound including a peptidyl moiety (e.g. a meditope) may form part of the first cavity, the second cavity, the hole region or any combination thereof. The non-CDR peptide binding region may be engineered into any appropriate antibody thereby forming an antibody domain (antigen binding domain) with the non-CDR peptide binding region. An antigen binding domain including a non-CDR peptide binding region is also referred to herein as meditope-enabled antibody, meditope-enabled domain or meditope-enabled antibody region. An antigen binding domain (e.g., antibody, antibody domain) including a non-CDR peptide binding domain provided herein (i.e., capable of forming a disulfide bridge with a protein compound (e.g., meditope)) is also referred to herein as “cysteine-meditope-enabled antibody”, “cysteine-meditope-enabled domain” or “cysteine-meditope-enabled antibody region.”

As described herein, the antigen binding domain provided herein includes a central hole and non-CDR binding region including a first cavity, a second cavity and a hole region. The first cavity provided herein is also referred to as a “meditope binding site.” In embodiments, the non-CDR peptide binding region includes framework region amino acid residues. In embodiments, the non-CDR peptide binding region includes FR residues of the heavy chain or the light chain. In embodiments, the non-CDR peptide binding region includes FR residues of the heavy chain and the light chain. In embodiments, the non-CDR peptide binding region includes a residue at a position corresponding to Kabat position 83, a residue at a position corresponding to Kabat position 30 or a residue at a position corresponding to Kabat position 52. In embodiments, the non-CDR peptide binding region includes a residue at a position corresponding to Kabat position 40, a residue at a position corresponding to Kabat position 41, a residue at a position corresponding to Kabat position 30, a residue at a position corresponding to Kabat position 52, a residue at a position corresponding to Kabat position 83, or a residue at a position corresponding to Kabat position 85. In embodiments, the non-CDR peptide binding region includes a residue at a position corresponding to Kabat position 40. In embodiments, the non-CDR peptide binding region includes a residue at a position corresponding to Kabat position 41. In embodiments, the non-CDR peptide binding region includes a residue at a position corresponding to Kabat position 30. In embodiments, the non-CDR peptide binding region includes a residue at a position corresponding to Kabat position 52. In embodiments, the non-CDR peptide binding region includes a residue at a position corresponding to Kabat position 83. In embodiments, the non-CDR peptide binding region includes a residue at a position corresponding to Kabat position 85. In embodiments, residues forming a non-CDR peptide binding region are described in published US application US20120301400 A1, which is hereby incorporate by reference in its entirety and for all purposes.

In embodiments, the non-CDR peptide binding region is formed by amino acid residues at positions 8, 9, 10, 38, 39, 40, 41, 42, 43, 44, 45, 82, 83, 84, 85, 86, 87, 99, 100, 101, 102, 103, 104, 105, 142, 162, 163, 164, 165, 166, 167, 168, and 173 of the VL region and 6, 9, 38, 39, 40, 41, 42, 43, 44, 45, 84, 86, 87, 88, 89, 90, 91, 103, 104, 105, 106, 107, 108, 111, 110, 147, 150, 151, 152, 173, 174, 175, 176, 177, 185, 186, and 187 of the VH region, according to Kabat numbering. In embodiments, the non-CDR peptide binding region has a light chain sequence including P8, V9 or 19, 110 or L10, Q38, R39, T40, N41 G42, S43, P44, R45, D82, 183, A84, D85, Y86, Y87, G99, A100, G101, T102, K103, L104, E105, R142, S162, V163, T164, E165, Q166, D167, S168, and/or Y173, according to Kabat numbering, and/or has a heavy chain having Q6, P9, R38, Q39, S40, P41, G42, K43, G44, L45, S84, D86, T87, A88, 189, Y90, Y91, W103, G104, Q105, G106, T107, L108, V111, T110, Y147, E150, P151, V152, T173, F174, C175, A176, V177, Y185, S186, and/or L187, according to Kabat numbering.

In embodiments, the non-CDR peptide binding region includes a Glu at position 83 of the VL region, according to Kabat numbering. In embodiments, the non-CDR peptide binding region includes a Thr or Ser at position 40 of the VH region, according to Kabat numbering. In embodiments, the non-CDR peptide binding region includes an Asn at position 41 of the VL region, according to Kabat numbering. In embodiments, the non-CDR peptide binding region includes an Asp or Asn at position 85 of the VL region, according to Kabat numbering.

The non-CDR peptide binding region provided herein includes a first cysteine, which forms a disulfide linkage with a thiol side chain amino acid included in the peptide compound provided herein, thereby covalently attaching the peptide compound to the antigen binding domain. In embodiments, the first cysteine forms part of the first set of amino acid residues (amino acid residues of the first cavity), the second set of amino acid residues (amino acid residues of the second cavity) or the third set of amino acid residues (amino acid residues of the hole region). Thus, in embodiments, the first set of amino acid residues includes the first cysteine at a position corresponding to Kabat position 102, 142 or 143 of the VL region. In embodiments, the first set of amino acid residues includes the first cysteine at a position corresponding to Kabat position 102 of the VL region. In embodiments, the first set of amino acid residues includes the first cysteine at a position corresponding to Kabat position 142 of the VL region. In embodiments, the first set of amino acid residues includes the first cysteine at a position corresponding to Kabat position 143 of the VL region.

In embodiments, the second set of amino acid residues includes the first cysteine at a position corresponding to Kabat position 208 or 158 of the VH region. In embodiments, the second set of amino acid residues includes the first cysteine at a position corresponding to Kabat position 208 of the VH region. In embodiments, the second set of amino acid residues includes the first cysteine at a position corresponding to Kabat position 158 of the VH region.

In embodiments, the third set of amino acid residues includes the first cysteine at a position corresponding to Kabat position 174 or 175 of the VH region. In embodiments, the third set of amino acid residues includes the first cysteine at a position corresponding to Kabat position 174 of the VH region. In embodiments, the third set of amino acid residues includes the first cysteine at a position corresponding to Kabat position 175 of the VH region.

The covalent complexes provided herein include an antigen binding domain (e.g., a Fab) covalently attached to a peptide compound (e. g., a peptide compound of formula (I) or formula (II)) through a disulfide linkage. A “disulfide linkage”, “disulfide bridge” or “disulfide bond” as provided herein refers to a covalent bond formed by reacting two thiol moieties. The first of the two reacting thiol moieties forms part of the antigen binding domain provided herein and the second thiol moiety forms part of the peptide compound provided herein. In embodiments, the covalent complex provided herein has the structure of R^{A}—S—S—R^{B}, wherein R^{A} is an antigen binding domain and R^{B} is a peptide compound. The disulfide linkage is formed between a cysteine of the antigen binding domain (first cysteine) and a thiol side chain amino acid (e.g., a cysteine or a substituted arginine) included in the peptide compound. Any of the first cysteines provided herein may form a disulfide linkage with any of the thiol side chain amino acids included in the peptide compound.

A “thiol side chain amino acid” as provided herein is an amino acid which includes a side chain with a sulfur atom, wherein the sulfur forms part of a disulfide linkage, and may also be referred to herein as a “sulfur-containing side chain amino acid.” A thiol side chain amino acid as referred to herein includes a sulfur atom derived from a reacted —SH substituent (i.e., a thiol group or thiol substituent which is a group or substituent including a thiol). Thus, the thiol side chain amino acid provided herein may also be referred to as sulfur amino acid side chain. The sulfur atom of a thiol side chain amino acid is formed through reaction of a side chain thiol group with a thiol group of a second reactant (e.g., the side chain thiol group of the first cysteine). In embodiments, the sulfur atom forms part of the side chain of an amino acid (e.g., a cysteine side chain). In embodiments, the sulfur atom forms part of a substituted amino acid side chain (e.g., a substituted arginine side chain). Where the sulfur atom forms part of a substituted amino acid side chain, the amino acid side chain may be substituted with a substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. In embodiments, the substituted amino acid side chain is substituted with octyl-thiol. In embodiments, the octyl-thiol has the formula:

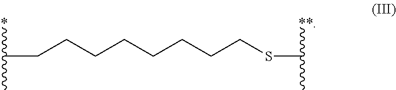

In formula (III) * denotes the attachment point with the amino acid side chain and ** denotes the point of attachment with the first cysteine. In embodiments, the substituted amino acid side chain is a substituted arginine side chain. In embodiments, the substituted arginine includes the compound of formula (III). In embodiments, the substituted arginine is an octyl-thiol-substituted arginine. In embodiments, the octyl-thiol-substituted arginine includes the compound of formula (III). In embodiments, the thiol side chain amino acid is a cysteine.

In embodiments, the peptide compound has the formula:

R¹-X0-X1-X2-X3-X4-X5-X6-X7-X8-X9-X10-X11-X12-R² (I)

In formula (I), X0 is Ser or null. X1 is Ser, Cys, Gly, β-alanine, diaminopropionic acid, β-azidoalanine, or null. X2 is Gln or null. X3 is Phe, Tyr, β,β′-diphenyl-Ala, His, Asp, 2-bromo-L-phenylalanine, 3-bromo-L-phenylalanine, 4-bromo-L-phenylalanine, Asn, Gln, a modified Phe, a hydratable carbonyl-containing residue, or a boronic acid-containing residue. X4 is Asp or Asn. X5 is Leu, β,β′-diphenyl-Ala, Phe, Trp, Tyr, a non-natural analog of phenylalanine, tryptophan, or tyrosine, a hydratable carbonyl-containing residue, or a boronic acid-containing residue. X6 is the thiol side chain amino acid or serine. X7 is the thiol side chain amino acid, Thr, or Ser. X8 is the thiol side chain amino acid, Arg, Ala, or an amino acid comprising a side chain of the formula -L^{3A}-L^{3B}-R³, wherein L^{3A} is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, L^{3B} is a chemical linker and R³ is a steric hindering chemical moiety. X9 is the thiol side chain amino acid, Arg or Ala. X10 is Leu, Gln, Glu, β,β′-diphenyl-Ala, Phe, Trp, Tyr; a non-natural analog of phenylalanine, tryptophan, or tyrosine, a hydratable carbonyl-containing residue, or a boronic acid-containing residue. X11 is the thiol side chain amino acid, Gln, Lys or Arg. X12 is Ser, Cys, Gly, 7-aminoheptanoic acid, β-alanine, diaminopropionic acid, propargylglycine, isoaspartic acid, or null. R¹ is null, -L^{10A}-L^{10B}-R¹⁰, an amino acid peptide sequence (also referred to herein as a peptidyl moiety) optionally substituted with -L^{10A}-L^{10B}-R¹⁰. R² is null, -L^{20A}-L^{20B}-R²⁰, an amino acid peptide sequence optionally substituted with -L^{20A}-L^{20B}-R²⁰. L^{10A} L^{10B}, L^{20A}, L^{20B} are independently a bond, a peptidyl linker, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. R¹⁰ and R²⁰ are independently a reactive moiety, a diagnostic moiety, a therapeutic moiety or a detectable moiety. X1 and X12 are optionally joined together to form a cyclic peptidyl moiety. R¹ and X11 are optionally joined together to form a cyclic peptidyl moiety.

In embodiments, X0 is Ser. In embodiments, X0 is null. In embodiments, X1 is Ser. In embodiments, X1 is Cys. In embodiments, X1 is Gly. In embodiments, X1 is β-alanine. In embodiments, X1 is diaminopropionic acid. In embodiments, X1 is β-azidoalanine. In embodiments, X1 is null. In embodiments, X2 is Gln. In embodiments, X2 is null. In embodiments, X3 is Phe. In embodiments, X3 is Tyr. In embodiments, X3 is β,β′-diphenyl-Ala. In embodiments, X3 is His. In embodiments, X3 is Asp. In embodiments, X3 is 2-bromo-L-phenylalanine. In embodiments, X3 is 3-bromo-L-phenylalanine. In embodiments, X3 is 4-bromo-L-phenylalanine. In embodiments, X3 is Asn. In embodiments, X3 is Gln. In embodiments, X3 is a modified Phe. In embodiments, X3 is a hydratable carbonyl-containing residue. In embodiments, X3 is a boronic acid-containing residue. In embodiments, X4 is Asp. In embodiments, X4 is Asn. In embodiments, X5 is Leu. In embodiments, X5 is β,β′-diphenyl-Ala. In embodiments, X5 is Phe. In embodiments, X5 is Trp. In embodiments, X5 is Tyr. In embodiments, X5 is a non-natural analog of phenylalanine. In embodiments, X5 is a non-natural analog of tryptophan. In embodiments, X5 is a non-natural analog of tyrosine. In embodiments, X5 is a hydratable carbonyl-containing residue. In embodiments, X5 is a boronic acid-containing residue. In embodiments, X6 is the thiol side chain amino acid. In embodiments, X6 is serine. In embodiments, X7 is the thiol side chain amino acid. In embodiments, X7 is Thr. In embodiments, X7 is Ser. In embodiments, X8 is the thiol side chain amino acid. In embodiments, X8 is Arg. In embodiments, X8 is Ala. In embodiments, X8 is an amino acid comprising a side chain of the formula -L^{3A}-L^{3B}-R³, wherein L^{3A} is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, L^{3B} is a chemical linker and R³ is a steric hindering chemical moiety. In embodiments, X9 is the thiol side chain amino acid. In embodiments, X9 is Arg. In embodiments, X9 is Ala. In embodiments, X10 is Leu. In embodiments, X10 is Gln. In embodiments, X10 is Glu. In embodiments, X10 is β,β′-diphenyl-Ala. In embodiments, X10 is Phe. In embodiments, X10 is Trp. In embodiments, X10 is Tyr. In embodiments, X10 is a non-natural analog of phenylalanine. In embodiments, X10 is a non-natural analog of tryptophan. In embodiments, X10 is a non-natural analog of tyrosine. In embodiments, X10 is a hydratable carbonyl-containing residue. In embodiments, X10 is a boronic acid-containing residue. In embodiments, X11 is the thiol side chain amino acid. In embodiments, X11 is Gln. In embodiments, X11 is Lys. In embodiments, X11 is Arg. In embodiments, X12 is Ser. In embodiments, X12 is Cys. In embodiments, X12 is Gly. In embodiments, X12 is 7-aminoheptanoic acid. In embodiments, X12 is β-alanine. In embodiments, X12 is diaminopropionic acid. In embodiments, X12 is propargylglycine. In embodiments, X12 is isoaspartic acid. In embodiments, X12 is null. In embodiments, R¹ is null. In embodiments, R¹ is -L^{10A}-L^{10B}-R¹⁰. In embodiments, R¹ is an amino acid peptide sequence (also referred to herein as a peptidyl moiety) optionally substituted with -L^{10A}-L^{10B}-R¹⁰. In embodiments, R² is null. In embodiments, R² is -L^{20A}-L^{20B}-R²⁰. In embodiments, R² is an amino acid peptide sequence optionally substituted with -L^{20A}-L²⁰-R²⁰ In embodiments, L^{10A} L^{10B}, L^{20A}, L^{20B} are independently a bond, a peptidyl linker, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. In embodiments, R¹⁰ and R²⁰ are independently a reactive moiety, a diagnostic moiety, a therapeutic moiety or a detectable moiety. In embodiments, R¹⁰ is a reactive moiety. In embodiments, R²⁰ is a reactive moiety. In embodiments, R¹⁰ is a diagnostic moiety. In embodiments, R²⁰ is a diagnostic moiety. In embodiments, R¹⁰ is a therapeutic moiety. In embodiments, R²⁰ is a therapeutic moiety. In embodiments, R¹⁰ is a detectable moiety. In embodiments, R²⁰ is a detectable moiety. In embodiments, X1 and X12 are optionally joined together to form a cyclic peptidyl moiety. In embodiments, R¹ and X11 are optionally joined together to form a cyclic peptidyl moiety.

In embodiments, the peptide compound has the formula:

R¹-Ser-X2-X3-X4-β,β′-diphenylAla-Cys-Thr-X8-Arg-X10-X11-Ser-R² (IA).

In formula (IA), X2 is Gln or null. X3 is Phe, Tyr, β,β′-diphenyl-Ala, His, Asp, 2-bromo-L-phenylalanine, 3-bromo-L-phenylalanine, 4-bromo-L-phenylalanine, Asn, Gln, a modified Phe, a hydratable carbonyl-containing residue, or a boronic acid-containing residue. X4 is Asp or Asn. X8 is the thiol side chain amino acid, Arg, Ala, or an amino acid comprising a side chain of the formula -L^{3A}-L^{3B}-R³, wherein L^{3A} is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, L^{3B} is a chemical linker and R³ is a steric hindering chemical moiety. X10 is Leu, Gln, Glu, β,β′-diphenyl-Ala, Phe, Trp, Tyr; a non-natural analog of phenylalanine, tryptophan, or tyrosine, a hydratable carbonyl-containing residue, or a boronic acid-containing residue. X11 is Gln, Lys or Arg.

In embodiments, X2 is Gln. In embodiments, X2 is null. In embodiments, X3 is Phe. In embodiments, X3 is Tyr. In embodiments, X3 is β,β′-diphenyl-Ala. In embodiments, X3 is His. In embodiments, X3 is Asp. In embodiments, X3 is 2-bromo-L-phenylalanine. In embodiments, X3 is 3-bromo-L-phenylalanine. In embodiments, X3 is 4-bromo-L-phenylalanine. In embodiments, X3 is Asn. In embodiments, X3 is Gln. In embodiments, X3 is a modified Phe. In embodiments, X3 is a hydratable carbonyl-containing residue. In embodiments, X3 is a boronic acid-containing residue. In embodiments, X4 is Asp. In embodiments, X4 is Asn. In embodiments, X8 is the thiol side chain amino acid. In embodiments, X8 is Arg. In embodiments, X8 is Ala. In embodiments, X8 is an amino acid comprising a side chain of the formula -L^{3A}-L^{3B}-R³, wherein L^{3A} is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, L^{3B} is a chemical linker and R³ is a steric hindering chemical moiety. In embodiments, X10 is Leu. In embodiments, X10 is Gln. In embodiments, X10 is Glu. In embodiments, X10 is β,β′-diphenyl-Ala. In embodiments, X10 is Phe. In embodiments, X10 is Trp. In embodiments, X10 is Tyr. In embodiments, X10 is a non-natural analog of phenylalanine. In embodiments, X10 is a non-natural analog of tryptophan. In embodiments, X10 is a non-natural analog of tyrosine. In embodiments, X10 is a hydratable carbonyl-containing residue. In embodiments, X10 is a boronic acid-containing residue. In embodiments, X11 is Gln. In embodiments, X11 is Lys. In embodiments, X11 is Arg.

In embodiments, the peptide compound has the formula:

R¹-Gln-X3-X4-β,β′-diphenylAla-Ser-Thr-Arg-X9-X10-Lys-Ser-R² (IB).

In formula (IB), X3 is Phe, Tyr, β,β′-diphenyl-Ala, His, Asp, 2-bromo-L-phenylalanine, 3-bromo-L-phenylalanine, 4-bromo-L-phenylalanine, Asn, Gln, a modified Phe, a hydratable carbonyl-containing residue, or a boronic acid-containing residue. X4 is Asp or Asn. X9 is Arg or Ala. X10 is Leu, Gln, Glu, β,β′-diphenyl-Ala, Phe, Trp, Tyr; a non-natural analog of phenylalanine, tryptophan, or tyrosine, a hydratable carbonyl-containing residue, or a boronic acid-containing residue.

In embodiments, X3 is Phe. In embodiments, X3 is Tyr. In embodiments, X3 is β,β′-diphenyl-Ala. In embodiments, X3 is His. In embodiments, X3 is Asp. In embodiments, X3 is 2-bromo-L-phenylalanine. In embodiments, X3 is 3-bromo-L-phenylalanine. In embodiments, X3 is 4-bromo-L-phenylalanine. In embodiments, X3 is Asn. In embodiments, X3 is Gln. In embodiments, X3 is a modified Phe. In embodiments, X3 is a hydratable carbonyl-containing residue. In embodiments, X3 is a boronic acid-containing residue. In embodiments, X4 is Asp. In embodiments, X4 is Asn. In embodiments, X9 is Arg. In embodiments, X9 is Ala. In embodiments, X10 is Leu. In embodiments, X10 is Gln. In embodiments, X10 is Glu. In embodiments, X10 is β,β′-diphenyl-Ala. In embodiments, X10 is Phe. In embodiments, X10 is Trp. In embodiments, X10 is Tyr. In embodiments, X10 is a non-natural analog of phenylalanine. In embodiments, X10 is a non-natural analog of tryptophan. In embodiments, X10 is a non-natural analog of tyrosine. In embodiments, X10 is a hydratable carbonyl-containing residue. In embodiments, X10 is a boronic acid-containing residue.

In embodiments, the peptide compound has the formula:

R¹-X0-X1-X2-X3-X4-X5-X6-X7-X8-X9-X10-X11-X12-X13-X14-X15-R² (II).

In formula (II), X0 is Ser or null. X1 is Ser, Cys, Gly, β-alanine, diaminopropionic acid, β-azidoalanine, or null. X2 is Gln or null. X3 is Phe, Tyr, β,β′-diphenyl-Ala, His, Asp, 2-bromo-L-phenylalanine, 3-bromo-L-phenylalanine, 4-bromo-L-phenylalanine, Asn, Gln, a modified Phe, a hydratable carbonyl-containing residue, or a boronic acid-containing residue. X4 is Asp or Asn. X5 is Leu, β,β′-diphenyl-Ala, Phe, Trp, Tyr, a non-natural analog of phenylalanine, tryptophan, or tyrosine, a hydratable carbonyl-containing residue, or a boronic acid-containing residue. X6 is Ser. X7 is the thiol side chain amino acid, Thr, or Ser. X8 is Arg, Ala, or an amino acid comprising a side chain of the formula -L^{3A}-L^{3B}-R³, wherein L^{3A} is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, L^{3B} is a chemical linker and R³ is a steric hindering chemical moiety. X9 is the thiol side chain amino acid, Arg or Ala. X10 is Leu, Gln, Glu, β,β′-diphenyl-Ala, Phe, Trp, Tyr; a non-natural analog of phenylalanine, tryptophan, or tyrosine, a hydratable carbonyl-containing residue, or a boronic acid-containing residue. X11 is the thiol side chain amino acid, Gln, Lys or Arg. X12 is Ser, Cys, Gly, 7-aminoheptanoic acid, β-alanine, diaminopropionic acid, propargylglycine, isoaspartic acid, or null. X13 is Gly or Ser. X14 and X15 are independently Gly, Ser, Ala or the thiol side chain amino acid. R¹ is null, -L^{10A}-L^{10B}-R¹⁰, an amino acid peptide sequence optionally substituted with -L^{10A}-L^{10B}-R¹⁰. R² is null, -L^{20A}-L^{20B}-R²⁰, an amino acid peptide sequence optionally substituted with -L^{20A}-L^{20B}-R²⁰. L^{10A} L^{10B}, L^{20A}, L^{20B} are independently a bond, a peptidyl linker, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. R¹⁰ and R²⁰ are independently a reactive moiety, a diagnostic moiety, a therapeutic moiety or a detectable moiety. X1 and X12 are optionally joined together to form a cyclic peptidyl moiety.

In embodiments, X0 is Ser. In embodiments, X0 is null. In embodiments, X1 is Ser. In embodiments, X1 is Cys. In embodiments, X1 is Gly. In embodiments, X1 is β-alanine. In embodiments, X1 is diaminopropionic acid. In embodiments, X1 is β-azidoalanine. In embodiments, X1 is null. In embodiments, X2 is Gln. In embodiments, X2 is null. In embodiments, X3 is Phe. In embodiments, X3 is Tyr. In embodiments, X3 is β,β′-diphenyl-Ala. In embodiments, X3 is His. In embodiments, X3 is Asp. In embodiments, X3 is 2-bromo-L-phenylalanine. In embodiments, X3 is 3-bromo-L-phenylalanine. In embodiments, X3 is 4-bromo-L-phenylalanine. In embodiments, X3 is Asn. In embodiments, X3 is Gln. In embodiments, X3 is a modified Phe. In embodiments, X3 is a hydratable carbonyl-containing residue. In embodiments, X3 is a boronic acid-containing residue. In embodiments, X4 is Asp. In embodiments, X4 is Asn. In embodiments, X5 is Leu. In embodiments, X5 is β,β′-diphenyl-Ala. In embodiments, X5 is Phe. In embodiments, X5 is Trp. In embodiments, X5 is Tyr. In embodiments, X5 is a non-natural analog of phenylalanine. In embodiments, X5 is a non-natural analog of tryptophan. In embodiments, X5 is a non-natural analog of tyrosine. In embodiments, X5 is a hydratable carbonyl-containing residue. In embodiments, X5 is a boronic acid-containing residue. In embodiments, X6 is Ser. In embodiments, X7 is the thiol side chain amino acid. In embodiments, X7 is Thr. In embodiments, X7 is Ser. In embodiments, X8 is Arg. In embodiments, X8 is Ala. In embodiments, X8 is an amino acid comprising a side chain of the formula -L^{3A}-L^{3B}-R³, wherein L^{3A} is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, L^{3B} is a chemical linker and R³ is a steric hindering chemical moiety. In embodiments, X9 is the thiol side chain amino acid. In embodiments, X9 is Arg. In embodiments, X9 is Ala. In embodiments, X10 is Leu. In embodiments, X10 is Gln. In embodiments, X10 is Glu. In embodiments, X10 is β,β′-diphenyl-Ala. In embodiments, X10 is Phe. In embodiments, X10 is Trp. In embodiments, X10 is Tyr. In embodiments, X10 is a non-natural analog of phenylalanine. In embodiments, X10 is a non-natural analog of tryptophan. In embodiments, X10 is a non-natural analog of tyrosine. In embodiments, X10 is a hydratable carbonyl-containing residue. In embodiments, X10 is a boronic acid-containing residue. In embodiments, X11 is the thiol side chain amino acid. In embodiments, X11 is Gln. In embodiments, X11 is Lys. In embodiments, X11 is Arg. In embodiments, X12 is Ser. In embodiments, X12 is Cys. In embodiments, X12 is Gly. In embodiments, X12 is 7-aminoheptanoic acid. In embodiments, X12 is β-alanine. In embodiments, X12 is diaminopropionic acid. In embodiments, X12 is propargylglycine. In embodiments, X12 is isoaspartic acid. In embodiments, X12 is null. In embodiments, X13 is Gly. In embodiments, X13 is Ser. X14 and X15 are independently Gly, Ser, Ala or the thiol side chain amino acid. In embodiments, X14 is Gly. In embodiments, X14 is Ser. In embodiments, X14 is Ala. In embodiments, X14 is the thiol side chain amino acid. In embodiments, X15 is Gly. In embodiments, X15 is Ser. In embodiments, X15 is Ala. In embodiments, X15 is the thiol side chain amino acid. In embodiments, R¹ is null. In embodiments, R¹ is -L^{10A}-L^{10B}-R¹⁰. In embodiments, R¹ is an amino acid peptide sequence optionally substituted with -L^{10A}-L^{10B}-R¹⁰. In embodiments, R² is null. In embodiments, R² is -L^{20A}-L^{20B}-R²⁰. In embodiments, R² is an amino acid peptide sequence optionally substituted with -L^{20A}-L^{20B}-R²⁰. In embodiments, L^{10A}, L^{10B}, L^{20A}, L^{20B} are independently a bond, a peptidyl linker, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. In embodiments, R¹⁰ and R²⁰ are independently a reactive moiety, a diagnostic moiety, a therapeutic moiety or a detectable moiety. In embodiments, R¹⁰ is a reactive moiety. In embodiments, R¹⁰ is a diagnostic moiety. In embodiments, R¹⁰ is a therapeutic moiety. In embodiments, R¹⁰ is a detectable moiety. In embodiments, R²⁰ is a reactive moiety. In embodiments, R²⁰ is a diagnostic moiety. In embodiments, R²⁰ is a therapeutic moiety. In embodiments, R²⁰ is a detectable moiety. In embodiments, X1 and X12 are optionally joined together to form a cyclic peptidyl moiety.

In embodiments, the peptide compound has the formula:

R¹—Ser-X2-Phe-X4-β,β′-diphenylAla-Ser-Thr-X8-Arg-Leu-Gln-Ser-X13-X14-X15-R² (IIA).

In formula (IIA), X2 is Gln or null. X4 is Asp or Asn. X8 is Arg, Ala, or an amino acid comprising a side chain of the formula -L^{3A}-L^{3B}-R³, wherein L^{3A} is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, L^{3B} is a chemical linker and R³ is a steric hindering chemical moiety. X13 is Gly or Ser. X14 and X15 are independently Gly, Ser, Ala or the thiol side chain amino acid.

In embodiments, X2 is Gln. In embodiments, X2 is null. In embodiments, X4 is Asp. In embodiments, X4 is Asn. In embodiments, X8 is Arg. In embodiments, X8 is Ala. In embodiments, X8 is an amino acid comprising a side chain of the formula -L^{3A}-L^{3B}-R³, wherein L^{3A} is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, L^{3B} is a chemical linker and R³ is a steric hindering chemical moiety. In embodiments, X13 is Gly. In embodiments, X13 is Ser. In embodiments, X14 and X15 are independently Gly, Ser, Ala or the thiol side chain amino acid. In embodiments, X14 is Gly. In embodiments, X14 is Ser. In embodiments, X14 is Ala. In embodiments, X14 is the thiol side chain amino acid.

In embodiments, R¹ of formula (I), (IA), (IB), (II) and (IIA) is substituted (e.g., substituted with a substituent group(s), a size-limited substituent or a lower substituent group(s)) or unsubstituted alkyl, substituted (e.g., substituted with a substituent group(s), a size-limited substituent or a lower substituent group(s)) or unsubstituted heteroalkyl, substituted (e.g., substituted with a substituent group(s), a size-limited substituent or a lower substituent group(s)) or unsubstituted cycloalkyl, substituted (e.g., substituted with a substituent group(s), a size-limited substituent or a lower substituent group(s)) or unsubstituted heterocycloalkyl, substituted (e.g., substituted with a substituent group(s), a size-limited substituent or a lower substituent group(s)) or unsubstituted aryl or substituted (e.g., substituted with a substituent group(s), a size-limited substituent or a lower substituent group(s)) or unsubstituted heteroaryl.

In embodiments, R¹ of formula (I), (IA), (IB), (II) and (IIA) is (IIA) is substituted or unsubstituted (e.g., C₁-C₂₀, C₁-C₁₀, C₁-C₅) alkyl, substituted or unsubstituted (e.g., 2 to 20 membered, 2 to 10 membered, 2 to 5 membered) heteroalkyl, substituted or unsubstituted (e.g., C₃-C₅, C₃-C₆, C₃-C₅) cycloalkyl, substituted or unsubstituted (e.g., 3 to 8 membered, 3 to 6 membered, 3 to 5 membered) heterocycloalkyl, substituted or unsubstituted (e.g., C₆-C₁₀, C₆-C₅, C₆-C₅) aryl or substituted or unsubstituted (e.g., 5 to 10 membered, 5 to 8 membered, 5 to 6 membered,) heteroaryl.

In embodiments, R¹ of formula (I), (IA), (IB), (II) and (IIA) is unsubstituted (e.g., C₁-C₂₀, C₁-C₁₀, C₁-C₅) alkyl, unsubstituted (e.g., 2 to 20 membered, 2 to 10 membered, 2 to 5 membered) heteroalkyl, unsubstituted (e.g., C₃-C₅, C₃-C₆, C₃-C₅) cycloalkyl, unsubstituted (e.g., 3 to 8 membered, 3 to 6 membered, 3 to 5 membered) heterocycloalkyl, unsubstituted (e.g., C₆-C₁₀, C₆-C₅, C₆-C₅) aryl or unsubstituted (e.g., 5 to 10 membered, 5 to 8 membered, 5 to 6 membered,) heteroaryl.

In embodiments, R² of formula (I), (IA), (IB), (II) and (IIA) is substituted (e.g., substituted with a substituent group(s), a size-limited substituent or a lower substituent group(s)) or unsubstituted alkyl, substituted (e.g., substituted with a substituent group(s), a size-limited substituent or a lower substituent group(s)) or unsubstituted heteroalkyl, substituted (e.g., substituted with a substituent group(s), a size-limited substituent or a lower substituent group(s)) or unsubstituted cycloalkyl, substituted (e.g., substituted with a substituent group(s), a size-limited substituent or a lower substituent group(s)) or unsubstituted heterocycloalkyl, substituted (e.g., substituted with a substituent group(s), a size-limited substituent or a lower substituent group(s)) or unsubstituted aryl or substituted (e.g., substituted with a substituent group(s), a size-limited substituent or a lower substituent group(s)) or unsubstituted heteroaryl.

In embodiments, R² of formula (I), (IA), (IB), (II) and (IIA) is (IIA) is substituted or unsubstituted (e.g., C₁-C₂₀, C₁-C₁₀, C₁-C₅) alkyl, substituted or unsubstituted (e.g., 2 to 20 membered, 2 to 10 membered, 2 to 5 membered) heteroalkyl, substituted or unsubstituted (e.g., C₃-C₅, C₃-C₆, C₃-C₅) cycloalkyl, substituted or unsubstituted (e.g., 3 to 8 membered, 3 to 6 membered, 3 to 5 membered) heterocycloalkyl, substituted or unsubstituted (e.g., C₆-C₁₀, C₆-C₅, C₆-C₅) aryl or substituted or unsubstituted (e.g., 5 to 10 membered, 5 to 8 membered, 5 to 6 membered,) heteroaryl.

In embodiments, R² of formula (I), (IA), (IB), (II) and (IIA) is unsubstituted (e.g., C₁-C₂₀, C₁-C₁₀, C₁-C₅) alkyl, unsubstituted (e.g., 2 to 20 membered, 2 to 10 membered, 2 to 5 membered) heteroalkyl, unsubstituted (e.g., C₃-C₅, C₃-C₆, C₃-C₅) cycloalkyl, unsubstituted (e.g., 3 to 8 membered, 3 to 6 membered, 3 to 5 membered) heterocycloalkyl, unsubstituted (e.g., C₆-C₁₀, C₆-C₅, C₆-C₅) aryl or unsubstituted (e.g., 5 to 10 membered, 5 to 8 membered, 5 to 6 membered,) heteroaryl.

In embodiments, R³ of formula (I), (IA), (IB), (II) and (IIA) is substituted (e.g., substituted with a substituent group(s), a size-limited substituent or a lower substituent group(s)) or unsubstituted alkyl, substituted (e.g., substituted with a substituent group(s), a size-limited substituent or a lower substituent group(s)) or unsubstituted heteroalkyl, substituted (e.g., substituted with a substituent group(s), a size-limited substituent or a lower substituent group(s)) or unsubstituted cycloalkyl, substituted (e.g., substituted with a substituent group(s), a size-limited substituent or a lower substituent group(s)) or unsubstituted heterocycloalkyl, substituted (e.g., substituted with a substituent group(s), a size-limited substituent or a lower substituent group(s)) or unsubstituted aryl or substituted (e.g., substituted with a substituent group(s), a size-limited substituent or a lower substituent group(s)) or unsubstituted heteroaryl.

In embodiments, R³ of formula (I), (IA), (IB), (II) and (IIA) is (IIA) is substituted or unsubstituted (e.g., C₁-C₂₀, C₁-C₁₀, C₁-C₅) alkyl, substituted or unsubstituted (e.g., 2 to 20 membered, 2 to 10 membered, 2 to 5 membered) heteroalkyl, substituted or unsubstituted (e.g., C₃-C₅, C₃-C₆, C₃-C₅) cycloalkyl, substituted or unsubstituted (e.g., 3 to 8 membered, 3 to 6 membered, 3 to 5 membered) heterocycloalkyl, substituted or unsubstituted (e.g., C₆-C₁₀, C₆-C₅, C₆-C₅) aryl or substituted or unsubstituted (e.g., 5 to 10 membered, 5 to 8 membered, 5 to 6 membered,) heteroaryl.

In embodiments, R³ of formula (I), (IA), (IB), (II) and (IIA) is unsubstituted (e.g., C₁-C₂₀, C₁-C₁₀, C₁-C₅) alkyl, unsubstituted (e.g., 2 to 20 membered, 2 to 10 membered, 2 to 5 membered) heteroalkyl, unsubstituted (e.g., C₃-C₅, C₃-C₆, C₃-C₅) cycloalkyl, unsubstituted (e.g., 3 to 8 membered, 3 to 6 membered, 3 to 5 membered) heterocycloalkyl, unsubstituted (e.g., C₆-C₁₀, C₆-C₅, C₆-C₅) aryl or unsubstituted (e.g., 5 to 10 membered, 5 to 8 membered, 5 to 6 membered,) heteroaryl.

In embodiments, R¹⁰ of formula (I), (IA), (IB), (II) and (IIA) is substituted (e.g., substituted with a substituent group(s), a size-limited substituent or a lower substituent group(s)) or unsubstituted alkyl, substituted (e.g., substituted with a substituent group(s), a size-limited substituent or a lower substituent group(s)) or unsubstituted heteroalkyl, substituted (e.g., substituted with a substituent group(s), a size-limited substituent or a lower substituent group(s)) or unsubstituted cycloalkyl, substituted (e.g., substituted with a substituent group(s), a size-limited substituent or a lower substituent group(s)) or unsubstituted heterocycloalkyl, substituted (e.g., substituted with a substituent group(s), a size-limited substituent or a lower substituent group(s)) or unsubstituted aryl or substituted (e.g., substituted with a substituent group(s), a size-limited substituent or a lower substituent group(s)) or unsubstituted heteroaryl.

In embodiments, R¹⁰ of formula (I), (IA), (IB), (II) and (IIA) is (IIA) is substituted or unsubstituted (e.g., C₁-C₂₀, C₁-C₁₀, C₁-C₅) alkyl, substituted or unsubstituted (e.g., 2 to 20 membered, 2 to 10 membered, 2 to 5 membered) heteroalkyl, substituted or unsubstituted (e.g., C₃-C₅, C₃-C₆, C₃-C₅) cycloalkyl, substituted or unsubstituted (e.g., 3 to 8 membered, 3 to 6 membered, 3 to 5 membered) heterocycloalkyl, substituted or unsubstituted (e.g., C₆-C₁₀, C₆-C₅, C₆-C₅) aryl or substituted or unsubstituted (e.g., 5 to 10 membered, 5 to 8 membered, 5 to 6 membered,) heteroaryl.

In embodiments, R¹⁰ of formula (I), (IA), (IB), (II) and (IIA) is unsubstituted (e.g., C₁-C₂₀, C₁-C₁₀, C₁-C₅) alkyl, unsubstituted (e.g., 2 to 20 membered, 2 to 10 membered, 2 to 5 membered) heteroalkyl, unsubstituted (e.g., C₃-C₅, C₃-C₆, C₃-C₅) cycloalkyl, unsubstituted (e.g., 3 to 8 membered, 3 to 6 membered, 3 to 5 membered) heterocycloalkyl, unsubstituted (e.g., C₆-C₁₀, C₆-C₅, C₆-C₅) aryl or unsubstituted (e.g., 5 to 10 membered, 5 to 8 membered, 5 to 6 membered,) heteroaryl.

In embodiments, R²⁰ of formula (I), (IA), (IB), (II) and (IIA) is substituted (e.g., substituted with a substituent group(s), a size-limited substituent or a lower substituent group(s)) or unsubstituted alkyl, substituted (e.g., substituted with a substituent group(s), a size-limited substituent or a lower substituent group(s)) or unsubstituted heteroalkyl, substituted (e.g., substituted with a substituent group(s), a size-limited substituent or a lower substituent group(s)) or unsubstituted cycloalkyl, substituted (e.g., substituted with a substituent group(s), a size-limited substituent or a lower substituent group(s)) or unsubstituted heterocycloalkyl, substituted (e.g., substituted with a substituent group(s), a size-limited substituent or a lower substituent group(s)) or unsubstituted aryl or substituted (e.g., substituted with a substituent group(s), a size-limited substituent or a lower substituent group(s)) or unsubstituted heteroaryl.

In embodiments, R²⁰ of formula (I), (IA), (IB), (II) and (IIA) is (IIA) is substituted or unsubstituted (e.g., C₁-C₂₀, C₁-C₁₀, C₁-C₅) alkyl, substituted or unsubstituted (e.g., 2 to 20 membered, 2 to 10 membered, 2 to 5 membered) heteroalkyl, substituted or unsubstituted (e.g., C₃-C₅, C₃-C₆, C₃-C₅) cycloalkyl, substituted or unsubstituted (e.g., 3 to 8 membered, 3 to 6 membered, 3 to 5 membered) heterocycloalkyl, substituted or unsubstituted (e.g., C₆-C₁₀, C₆-C₅, C₆-C₅) aryl or substituted or unsubstituted (e.g., 5 to 10 membered, 5 to 8 membered, 5 to 6 membered,) heteroaryl.

In embodiments, R²⁰ of formula (I), (IA), (IB), (II) and (IIA) is unsubstituted (e.g., C₁-C₂₀, C₁-C₁₀, C₁-C₅) alkyl, unsubstituted (e.g., 2 to 20 membered, 2 to 10 membered, 2 to 5 membered) heteroalkyl, unsubstituted (e.g., C₃-C₅, C₃-C₆, C₃-C₅) cycloalkyl, unsubstituted (e.g., 3 to 8 membered, 3 to 6 membered, 3 to 5 membered) heterocycloalkyl, unsubstituted (e.g., C₆-C₁₀, C₆-C₅, C₆-C₅) aryl or unsubstituted (e.g., 5 to 10 membered, 5 to 8 membered, 5 to 6 membered,) heteroaryl.

L^{3A} of formula (I), (IA), (IB), (II) and (IIA) may be —O—, —S—, —S(O)₂—, —C(O)—, —C(O)O—, —C(O)NH—, —S(O)₂NH—, —NH—, —N═CH—, —NHC(O)NH—, substituted (e.g., substituted with a substituent group(s), a size-limited substituent or a lower substituent group(s)) or unsubstituted alkylene, substituted (e.g., substituted with a substituent group(s), a size-limited substituent or a lower substituent group(s)) or unsubstituted heteroalkylene, substituted (e.g., substituted with a substituent group(s), a size-limited substituent or a lower substituent group(s)) or unsubstituted cycloalkylene, substituted (e.g., substituted with a substituent group(s), a size-limited substituent or a lower substituent group(s)) or unsubstituted heterocycloalkylene, substituted (e.g., substituted with a substituent group(s), a size-limited substituent or a lower substituent group(s)) or unsubstituted arylene or substituted (e.g., substituted with a substituent group(s), a size-limited substituent or a lower substituent group(s)) or unsubstituted heteroarylene.

L^{3A} of formula (I), (IA), (IB), (II) and (IIA) may be —O—, —S—, —C(O)—, —C(O)O—, —C(O)NH—, —S(O)₂NH—, —NH—, —NHC(O)NH—, substituted or unsubstituted (e.g., C₁-C₂₀, C₁-C₁₀, C₁-C₅) alkylene, substituted or unsubstituted (e.g., 2 to 20 membered, 2 to 10 membered, 2 to 5 membered) heteroalkylene, substituted or unsubstituted (e.g., C₃-C₅, C₃-C₆, C₃-C₅) cycloalkylene, substituted or unsubstituted (e.g., 3 to 8 membered, 3 to 6 membered, 3 to 5 membered) heterocycloalkylene, substituted or unsubstituted (e.g., C₆-C₁₀, C₆-C₅, C₆-C₅) arylene or substituted or unsubstituted (e.g., to 10 membered, 5 to 8 membered, 5 to 6 membered,) heteroarylene.

L^{3A} of formula (I), (IA), (IB), (II) and (IIA) may be —O—, —S—, —C(O)—, —C(O)O—, —C(O)NH—, —S(O)₂NH—, —NH—, —NHC(O)NH—, unsubstituted (e.g., C₁-C₂₀, C₁-C₁₀, C₁-C₅) alkylene, unsubstituted (e.g., 2 to 20 membered, 2 to 10 membered, 2 to 5 membered) heteroalkylene, unsubstituted (e.g., C₃-C₅, C₃-C₆, C₃-C₅) cycloalkylene, unsubstituted (e.g., 3 to 8 membered, 3 to 6 membered, 3 to 5 membered) heterocycloalkylene, unsubstituted (e.g., C₆-C₁₀, C₆-C₅, C₆-C₅) arylene unsubstituted (e.g., 5 to 10 membered, 5 to 8 membered, 5 to 6 membered,) heteroarylene.

L^{3B} of formula (I), (IA), (IB), (II) and (IIA) is a chemical linker. The chemical linker provided herein may be a covalent or noncovalent linker. The chemical linker provided herein may include a chemically reactive functional group to react with a second chemically reactive functional group, thereby forming a covalent linker. A chemical linker as referred to herein may include the resulting linker formed by reacting two reactive groups (moieties), e.g., a covalent reactive group as described herein (e.g., alkyne, thiol, azide, maleimide). In embodiments, the chemical linker is a 1,3 triazole linker (i.e., a linker comprising a 1,3-triazolene linker moiety, e.g., in combination with alkyl (substituted or unsubstituted), amide, ester, sulfonamide and the like, including combinations thereof). The linkers provided herein may be covalently attached to the non-CDR peptide binding region or the steric hindering chemical moiety (R³) applying methods well known in the art and compatible with the composition of the complex provided herein. The linker provided herein may include the conjugated product of reactive groups, at the point of attachment to e.g., the non-CDR peptide binding region or the steric hindering chemical moiety. Thus, the linker provided herein may be polyvalent and/or may be formed by conjugate chemistry techniques. Non-limiting examples of linkers useful for the compositions and methods provided herein are linkers that include alkyl groups (including substituted alkyl groups and alkyl groups containing heteroatom moieties and short alkyl groups), ester groups, amide groups, amine groups, epoxy groups and/or ethylene glycol or derivatives thereof. The linkers provided herein may include a sulfone group, forming sulfonamide, an ester group and/or an ether group (e.g., triethyl ether).

In embodiments, the chemical linker provided herein is a cleavable peptide linker, including a protease cleavage site. A “cleavage site” as used herein, refers to a recognizable site for cleavage of a portion of a linker described herein. Thus, a cleavage site may be found in the sequence of a cleavable peptide linker as described herein, including embodiments thereof. In embodiments, the cleavage site is an amino acid sequence that is recognized and cleaved by a cleaving agent (e.g., a peptidyl sequence). Exemplary cleaving agents include proteins, enzymes, DNAzymes, RNAzymes, metals, acids, and bases. In embodiments, the protease cleavage site is a tumor-associated protease cleavage site. A “tumor-associated protease cleavage site” as provided herein is an amino acid sequence recognized by a protease, whose expression is specific for a tumor cell or tumor cell environment thereof. In embodiments, the protease cleavage site is a matrix metalloprotease (MMP) cleavage site, a disintegrin and metalloprotease domain-containing (ADAM) metalloprotease cleavage site, a prostate specific antigen (PSA) protease cleavage site, a urokinase-type plasminogen activator (uPA) protease cleavage site, a membrane type serine protease 1 (MT-SP1) protease cleavage site or a legumain protease cleavage site. In embodiments, the matrix metalloprotease (MMP) cleavage site is a MMP 9 cleavage site, a MMP 13 cleavage site or a MMP 2 cleavage site. In embodiments, the disintegrin and metalloprotease domain-containing (ADAM) metalloprotease cleavage site is a ADAM 9 metalloprotease cleavage site, a ADAM 10 metalloprotease cleavage site or a ADAM 17 metalloprotease cleavage site.

The chemical linker as provided herein (e.g. L^{3B}), may be —O—, —S—, —C(O)—, —C(O)O—, —C(O)NH—, —S(O)₂NH—, —NH—, —NHC(O)NH—, substituted (e.g., substituted with a substituent group, a size-limited substituent or a lower substituent group) or unsubstituted alkylene, substituted (e.g., substituted with a substituent group, a size-limited substituent or a lower substituent group) or unsubstituted heteroalkylene, substituted (e.g., substituted with a substituent group, a size-limited substituent or a lower substituent group) or unsubstituted cycloalkylene, substituted (e.g., substituted with a substituent group, a size-limited substituent or a lower substituent group) or unsubstituted heterocycloalkylene, substituted (e.g., substituted with a substituent group, a size-limited substituent or a lower substituent group) or unsubstituted arylene or substituted (e.g., substituted with a substituent group, a size-limited substituent or a lower substituent group) or unsubstituted heteroarylene.

The chemical linker as provided herein (e.g. L^{3B}), may be —O—, —S—, —C(O)—, —C(O)O—, —C(O)NH—, —S(O)₂NH—, —NH—, —NHC(O)NH—, substituted or unsubstituted (e.g., C₁-C₂₀, C₁-C₁₀, C₁-C₅) alkylene, substituted or unsubstituted (e.g., 2 to 20 membered, 2 to 10 membered, 2 to 5 membered) heteroalkylene, substituted or unsubstituted (e.g., C₃-C₅, C₃-C₆, C₃-C₅) cycloalkylene, substituted or unsubstituted (e.g., 3 to 8 membered, 3 to 6 membered, 3 to 5 membered) heterocycloalkylene, substituted or unsubstituted (e.g., C₆-C₁₀, C₆-C₅, C₆-C₅) arylene or substituted or unsubstituted (e.g., 5 to 10 membered, 5 to 8 membered, 5 to 6 membered,) heteroarylene.

The chemical linker as provided herein (e.g. L^{3B}), may be —O—, —S—, —C(O)—, —C(O)O—, —C(O)NH—, —S(O)₂NH—, —NH—, —NHC(O)NH—, unsubstituted (e.g., C₁-C₂₀, C₁-C₁₀, C₁-C₅) alkylene, unsubstituted (e.g., 2 to 20 membered, 2 to 10 membered, 2 to 5 membered) heteroalkylene, unsubstituted (e.g., C₃-C₅, C₃-C₆, C₃-C₅) cycloalkylene, unsubstituted (e.g., 3 to 8 membered, 3 to 6 membered, 3 to 5 membered) heterocycloalkylene, unsubstituted (e.g., C₆-C₁₀, C₆-C₅, C₆-C₅) arylene or unsubstituted (e.g., 5 to 10 membered, 5 to 8 membered, 5 to 6 membered,) heteroarylene.

In embodiments, the chemical linker (L^{3B}) is a covalent linker. In embodiments, the chemical linker (L^{3B}) is a PEG linker. In embodiments, the chemical linker (L^{3B}) is a hydrocarbon linker. In embodiments, the chemical linker (L^{3B}) is a cleavable peptide linker.

L^{10A}, L^{10B}, L^{20A}, and/or L^{20B} of formula (I), (IA), (IB), (II) and (IIA) may independently be a bond, a peptidyl linker, —O—, —S—, —C(O)—, —C(O)O—, —C(O)NH—, —S(O)₂NH—, —NH—, —NHC(O)NH—, substituted (e.g., substituted with a substituent group, a size-limited substituent or a lower substituent group) or unsubstituted alkylene, substituted (e.g., substituted with a substituent group, a size-limited substituent or a lower substituent group) or unsubstituted heteroalkylene, substituted (e.g., substituted with a substituent group, a size-limited substituent or a lower substituent group) or unsubstituted cycloalkylene, substituted (e.g., substituted with a substituent group, a size-limited substituent or a lower substituent group) or unsubstituted heterocycloalkylene, substituted (e.g., substituted with a substituent group, a size-limited substituent or a lower substituent group) or unsubstituted arylene or substituted (e.g., substituted with a substituent group, a size-limited substituent or a lower substituent group) or unsubstituted heteroarylene.

In embodiments, L^{10A}, L^{10B}, L^{20A}, and/or L^{20B} are independently a bond, a peptidyl linker, —O—, —S—, —C(O)—, —C(O)O—, —C(O)NH—, —S(O)₂NH—, —NH—, —NHC(O)NH—, substituted or unsubstituted (e.g., C₁-C₂₀, C₁-C₁₀, C₁-C₅) alkylene, substituted or unsubstituted (e.g., 2 to 20 membered, 2 to 10 membered, 2 to 5 membered) heteroalkylene, substituted or unsubstituted (e.g., C₃-C₅, C₃-C₆, C₃-C₅) cycloalkylene, substituted or unsubstituted (e.g., 3 to 8 membered, 3 to 6 membered, 3 to 5 membered) heterocycloalkylene, substituted or unsubstituted (e.g., C₆-C₁₀, C₆-C₅, C₆-C₅) arylene or substituted or unsubstituted (e.g., 5 to 10 membered, 5 to 8 membered, 5 to 6 membered,) heteroarylene.

In embodiments, L^{10A}, L^{10B}, L^{20A}, and/or L^{20B} are independently a bond, a peptidyl linker, —O—, —S—, —C(O)—, —C(O)O—, —C(O)NH—, —S(O)₂NH—, —NH—, —NHC(O)NH—, unsubstituted (e.g., C₁-C₂₀, C₁-C₁₀, C₁-C₅) alkylene, unsubstituted (e.g., 2 to 20 membered, 2 to 10 membered, 2 to 5 membered) heteroalkylene, unsubstituted (e.g., C₃-C₅, C₃-C₆, C₃-C₅) cycloalkylene, unsubstituted (e.g., 3 to 8 membered, 3 to 6 membered, 3 to 5 membered) heterocycloalkylene, unsubstituted (e.g., C₆-C₁₀, C₆-C₅, C₆-C₅) arylene or unsubstituted (e.g., 5 to 10 membered, 5 to 8 membered, 5 to 6 membered,) heteroarylene.

L^{10A} of formula (I), (IA), (IB), (II) and (IIA) may be a bond, a peptidyl linker, —O—, —S—, —C(O)—, —C(O)O—, —C(O)NH—, —S(O)₂NH—, —NH—, —NHC(O)NH—, substituted (e.g., substituted with a substituent group, a size-limited substituent or a lower substituent group) or unsubstituted alkylene, substituted (e.g., substituted with a substituent group, a size-limited substituent or a lower substituent group) or unsubstituted heteroalkylene, substituted (e.g., substituted with a substituent group, a size-limited substituent or a lower substituent group) or unsubstituted cycloalkylene, substituted (e.g., substituted with a substituent group, a size-limited substituent or a lower substituent group) or unsubstituted heterocycloalkylene, substituted (e.g., substituted with a substituent group, a size-limited substituent or a lower substituent group) or unsubstituted arylene or substituted (e.g., substituted with a substituent group, a size-limited substituent or a lower substituent group) or unsubstituted heteroarylene.

In embodiments, L^{10A} is a bond, a peptidyl linker, —O—, —S—, —C(O)—, —C(O)O—, —C(O)NH—, —S(O)₂NH—, —NH—, —NHC(O)NH—, substituted or unsubstituted (e.g., C₁-C₂₀, C₁-C₁₀, C₁-C₅) alkylene, substituted or unsubstituted (e.g., 2 to 20 membered, 2 to 10 membered, 2 to 5 membered) heteroalkylene, substituted or unsubstituted (e.g., C₃-C₅, C₃-C₆, C₃-C₅) cycloalkylene, substituted or unsubstituted (e.g., 3 to 8 membered, 3 to 6 membered, 3 to 5 membered) heterocycloalkylene, substituted or unsubstituted (e.g., C₆-C₁₀, C₆-C₅, C₆-C₅) arylene or substituted or unsubstituted (e.g., to 10 membered, 5 to 8 membered, 5 to 6 membered,) heteroarylene.

In embodiments, L^{10A} is a bond, a peptidyl linker, —O—, —S—, —C(O)—, —C(O)O—, —C(O)NH—, —S(O)₂NH—, —NH—, —NHC(O)NH—, unsubstituted (e.g., C₁-C₂₀, C₁-C₁₀, C₁-C₅) alkylene, unsubstituted (e.g., 2 to 20 membered, 2 to 10 membered, 2 to 5 membered) heteroalkylene, unsubstituted (e.g., C₃-C₅, C₃-C₆, C₃-C₅) cycloalkylene, unsubstituted (e.g., 3 to 8 membered, 3 to 6 membered, 3 to 5 membered) heterocycloalkylene, unsubstituted (e.g., C₆-C₁₀, C₆-C₅, C₆-C₅) arylene or unsubstituted (e.g., 5 to 10 membered, 5 to 8 membered, 5 to 6 membered,) heteroarylene.

L^{10B} of formula (I), (IA), (IB), (II) and (IIA) may be a bond, a peptidyl linker, —O—, —S—, —C(O)—, —C(O)O—, —C(O)NH—, —S(O)₂NH—, —NH—, —NHC(O)NH—, substituted (e.g., substituted with a substituent group, a size-limited substituent or a lower substituent group) or unsubstituted alkylene, substituted (e.g., substituted with a substituent group, a size-limited substituent or a lower substituent group) or unsubstituted heteroalkylene, substituted (e.g., substituted with a substituent group, a size-limited substituent or a lower substituent group) or unsubstituted cycloalkylene, substituted (e.g., substituted with a substituent group, a size-limited substituent or a lower substituent group) or unsubstituted heterocycloalkylene, substituted (e.g., substituted with a substituent group, a size-limited substituent or a lower substituent group) or unsubstituted arylene or substituted (e.g., substituted with a substituent group, a size-limited substituent or a lower substituent group) or unsubstituted heteroarylene.

In embodiments, L^{10B} is a bond, a peptidyl linker, —O—, —S—, —C(O)—, —C(O)O—, —C(O)NH—, —S(O)₂NH—, —NH—, —NHC(O)NH—, substituted or unsubstituted (e.g., C₁-C₂₀, C₁-C₁₀, C₁-C₅) alkylene, substituted or unsubstituted (e.g., 2 to 20 membered, 2 to 10 membered, 2 to 5 membered) heteroalkylene, substituted or unsubstituted (e.g., C₃-C₅, C₃-C₆, C₃-C₅) cycloalkylene, substituted or unsubstituted (e.g., 3 to 8 membered, 3 to 6 membered, 3 to 5 membered) heterocycloalkylene, substituted or unsubstituted (e.g., C₆-C₁₀, C₆-C₅, C₆-C₅) arylene or substituted or unsubstituted (e.g., to 10 membered, 5 to 8 membered, 5 to 6 membered,) heteroarylene.

In embodiments, L^{10B} is a bond, a peptidyl linker, —O—, —S—, —C(O)—, —C(O)O—, —C(O)NH—, —S(O)₂NH—, —NH—, —NHC(O)NH—, unsubstituted (e.g., C₁-C₂₀, C₁-C₁₀, C₁-C₅) alkylene, unsubstituted (e.g., 2 to 20 membered, 2 to 10 membered, 2 to 5 membered) heteroalkylene, unsubstituted (e.g., C₃-C₅, C₃-C₆, C₃-C₅) cycloalkylene, unsubstituted (e.g., 3 to 8 membered, 3 to 6 membered, 3 to 5 membered) heterocycloalkylene, unsubstituted (e.g., C₆-C₁₀, C₆-C₅, C₆-C₅) arylene or unsubstituted (e.g., 5 to 10 membered, 5 to 8 membered, 5 to 6 membered,) heteroarylene.

L^{20A} of formula (I), (IA), (IB), (II) and (IIA) may be a bond, a peptidyl linker, —O—, —S—, —C(O)—, —C(O)O—, —C(O)NH—, —S(O)₂NH—, —NH—, —NHC(O)NH—, substituted (e.g., substituted with a substituent group, a size-limited substituent or a lower substituent group) or unsubstituted alkylene, substituted (e.g., substituted with a substituent group, a size-limited substituent or a lower substituent group) or unsubstituted heteroalkylene, substituted (e.g., substituted with a substituent group, a size-limited substituent or a lower substituent group) or unsubstituted cycloalkylene, substituted (e.g., substituted with a substituent group, a size-limited substituent or a lower substituent group) or unsubstituted heterocycloalkylene, substituted (e.g., substituted with a substituent group, a size-limited substituent or a lower substituent group) or unsubstituted arylene or substituted (e.g., substituted with a substituent group, a size-limited substituent or a lower substituent group) or unsubstituted heteroarylene.

In embodiments, L^{20A} is a bond, a peptidyl linker, —O—, —S—, —C(O)—, —C(O)O—, —C(O)NH—, —S(O)₂NH—, —NH—, —NHC(O)NH—, substituted or unsubstituted (e.g., C₁-C₂₀, C₁-C₁₀, C₁-C₅) alkylene, substituted or unsubstituted (e.g., 2 to 20 membered, 2 to 10 membered, 2 to 5 membered) heteroalkylene, substituted or unsubstituted (e.g., C₃-C₅, C₃-C₆, C₃-C₅) cycloalkylene, substituted or unsubstituted (e.g., 3 to 8 membered, 3 to 6 membered, 3 to 5 membered) heterocycloalkylene, substituted or unsubstituted (e.g., C₆-C₁₀, C₆-C₅, C₆-C₅) arylene or substituted or unsubstituted (e.g., to 10 membered, 5 to 8 membered, 5 to 6 membered,) heteroarylene.

In embodiments, L^{20A} is a bond, a peptidyl linker, —O—, —S—, —C(O)—, —C(O)O—, —C(O)NH—, —S(O)₂NH—, —NH—, —NHC(O)NH—, unsubstituted (e.g., C₁-C₂₀, C₁-C₁₀, C₁-C₅) alkylene, unsubstituted (e.g., 2 to 20 membered, 2 to 10 membered, 2 to 5 membered) heteroalkylene, unsubstituted (e.g., C₃-C₅, C₃-C₆, C₃-C₅) cycloalkylene, unsubstituted (e.g., 3 to 8 membered, 3 to 6 membered, 3 to 5 membered) heterocycloalkylene, unsubstituted (e.g., C₆-C₁₀, C₆-C₅, C₆-C₅) arylene or unsubstituted (e.g., 5 to 10 membered, 5 to 8 membered, 5 to 6 membered,) heteroarylene.

L^{20B} of formula (I), (IA), (IB), (II) and (IIA) may be a bond, a peptidyl linker, —O—, —S—, —C(O)—, —C(O)O—, —C(O)NH—, —S(O)₂NH—, —NH—, —NHC(O)NH—, substituted (e.g., substituted with a substituent group, a size-limited substituent or a lower substituent group) or unsubstituted alkylene, substituted (e.g., substituted with a substituent group, a size-limited substituent or a lower substituent group) or unsubstituted heteroalkylene, substituted (e.g., substituted with a substituent group, a size-limited substituent or a lower substituent group) or unsubstituted cycloalkylene, substituted (e.g., substituted with a substituent group, a size-limited substituent or a lower substituent group) or unsubstituted heterocycloalkylene, substituted (e.g., substituted with a substituent group, a size-limited substituent or a lower substituent group) or unsubstituted arylene or substituted (e.g., substituted with a substituent group, a size-limited substituent or a lower substituent group) or unsubstituted heteroarylene.

In embodiments, L^{20B} is a bond, a peptidyl linker, —O—, —S—, —C(O)—, —C(O)O—, —C(O)NH—, —S(O)₂NH—, —NH—, —NHC(O)NH—, substituted or unsubstituted (e.g., C₁-C₂₀, C₁-C₁₀, C₁-C₅) alkylene, substituted or unsubstituted (e.g., 2 to 20 membered, 2 to 10 membered, 2 to 5 membered) heteroalkylene, substituted or unsubstituted (e.g., C₃-C₅, C₃-C₆, C₃-C₅) cycloalkylene, substituted or unsubstituted (e.g., 3 to 8 membered, 3 to 6 membered, 3 to 5 membered) heterocycloalkylene, substituted or unsubstituted (e.g., C₆-C₁₀, C₆-C₅, C₆-C₅) arylene or substituted or unsubstituted (e.g., to 10 membered, 5 to 8 membered, 5 to 6 membered,) heteroarylene.

In embodiments, L^{20B} is a bond, a peptidyl linker, —O—, —S—, —C(O)—, —C(O)O—, —C(O)NH—, —S(O)₂NH—, —NH—, —NHC(O)NH—, unsubstituted (e.g., C₁-C₂₀, C₁-C₁₀, C₁-C₅) alkylene, unsubstituted (e.g., 2 to 20 membered, 2 to 10 membered, 2 to 5 membered) heteroalkylene, unsubstituted (e.g., C₃-C₅, C₃-C₆, C₃-C₅) cycloalkylene, unsubstituted (e.g., 3 to 8 membered, 3 to 6 membered, 3 to 5 membered) heterocycloalkylene, unsubstituted (e.g., C₆-C₁₀, C₆-C₅, C₆-C₅) arylene or unsubstituted (e.g., 5 to 10 membered, 5 to 8 membered, 5 to 6 membered,) heteroarylene.

R³ of formula (I), (IA), (IB), (II) and (IIA) is a steric hindering chemical moiety. A “steric hindering chemical moiety” provided herein is a moiety which is sterically hindered to pass through the central hole forming part of the antigen binding domain. The steric hindrance occurs between the steric hindering chemical moiety and the amino acids lining the central hole, thereby facilitating the mechanical interlock. Thus, the steric hindering chemical moiety is sufficient in size, dimension or volume to create steric hindrance (“plug”), thereby significantly decreasing (e.g., inhibiting or preventing) the ability of the steric hindering chemical moiety to pass through the hole towards the side of the antigen binding domain which forms the first cavity. In embodiments, the longest diameter of the central hole (e.g., the longest distance across the central hole measured from amino acid residue to amino acid residue by crystal structure) in which the steric hindering chemical moiety could pass is shorter than the longest dimension (e.g., diameter) of the steric hindering chemical moiety (also referred to herein as R³). In embodiments, the central hole (e.g., the longest diameter of the hole as measure in a crystal structure) is from about 3 to about 10 Å in size (e.g., in length, in diameter). In embodiments, the longest dimension of the steric hindering chemical moiety is more than about 3 to about 10 Å in size. For example, where the central hole is 8 Å in size (e.g., the longest diameter of the hole as measure in a crystal structure or diameter), the steric hindering chemical moiety is more than about 8 Å in size (i.e., the longest dimension is more than about 8 Å in size). Binding of the steric hindering chemical moiety to the remainder of the peptide compound is typically accomplished using click chemistry. In embodiments, a chemically reactive functional group (e.g., alkyne) is present on the steric hindering chemical moiety that is reacted with a conjugate (click) chemistry present on the chemical linker to be reacted. In embodiments, the steric hindering chemical moiety is a substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. In embodiments, the steric hindering chemical moiety is a substituted or unsubstituted diphenyl. The steric hindering chemical moiety may or may not bind or interact with the non-CDR peptide binding region. In embodiments, the steric hindering chemical moiety does not bind or interact with the non-CDR peptide binding region.

The peptide compounds of formula (I), (IA), (IB), (II) and (IIA) provided herein may include a therapeutic or diagnostic agent. Thus, in embodiments, R¹⁰ and R²⁰ are independently a therapeutic agent, a diagnostic agent or a detectable agent. The therapeutic agent, diagnostic agent or detectable agent (also referred to herein as R¹⁰ and/or R²⁰) may be attached through a non-covalent or covalent linker (also referred to herein as L^{10A} L^{10B}, L^{20A}, or L^{20B}) to the peptide compound provided herein including embodiments thereof.

The peptide compounds of formula (I), (IA), (IB), (II) and (IIA) provided herein may include a click chemistry reactive moiety. Thus, in embodiments, R¹, R², R¹⁰ and R²⁰ are independently a reactive moiety. The reactive moiety (also referred to herein as R¹, R², R¹⁰ and/or R²⁰) may be attached through a non-covalent or covalent linker (also referred to herein as L^{10A} L^{10B}L^{20A}, or L^{20B}) to the peptide compound provided herein including embodiments thereof. In embodiments, R¹, R², R¹⁰ and R²⁰ are independently a reactive moiety and the reactive moiety is reacted with a second reactive moiety (second R¹, second R², second R¹⁰ or a second R²⁰) of a second peptide compound provided herein including embodiments thereof. Thus, in embodiments, R¹ is a reactive moiety. In embodiments, R² is a reactive moiety. In embodiments, R¹⁰ is a reactive moiety. In embodiments, R²⁰ is a reactive moiety. The reactive moiety (e.g., R¹, R², R¹⁰ and R²⁰) of the first peptide compound provided herein may react with a second reactive moiety (e.g., R¹, R², R¹⁰ and R²⁰) of the second peptide compound (e.g., through click chemistry) thereby forming a covalent linker covalently connecting the first peptide compound with the second peptide compound. In embodiments, the first peptide compound is a peptide compound as provided herein including embodiments thereof. In embodiments, the second peptide compound is a peptide compound as provided herein including embodiments thereof. In embodiments, the first peptide compound forms a first covalent complex with (e.g., is covalently bound through a disulfide linkage to) a first antigen binding domain (e.g., an anti-CD3 antigen binding domain, an anti-CD16 antigen binding domain) and the second peptide compound forms a second covalent complex with (e.g., is covalently bound through a disulfide linkage to) a second antigen binding domain (e.g., an anti-HER2 antigen binding domain) thereby forming a multi-specific antigen binding complex. Therefore, the compositions provided, herein may be used to form antigen binding conjugates capable of binding two or more antigens, wherein the two or more antigens may be chemically different.

The peptide compounds provided herein (e.g., peptide compounds of formula (I), (IA), (IB), (II) and (IIA)) may include a therapeutic moiety (also referred to herein as R¹⁰ or R²⁰). The therapeutic moiety may be a protein moiety. In embodiments, the protein moiety is an antibody variant moiety. In embodiments, the antibody variant moiety is a variable heavy chain nanobody moiety (a nanobody moiety including a variable heavy chain domain). In embodiments, the antibody variant moiety is a variable light chain nanobody moiety (a nanobody moiety including a variable light chain domain). In embodiments, the antibody variant moiety is an anti-CD16 nanobody moiety. In embodiments, the antibody variant moiety is an anti-HER2 affibody moiety. The term “moiety” as referred to herein is a protein or peptide (e.g., nanobody) attached to the remainder of the molecule (e.g. R¹, R², or -L^{3A}-L^{3B}-R³ of the peptide compound of formula (I), (IA), (IB), (II) or (IIA)). The therapeutic moiety (also referred to herein as R¹⁰ or R²⁰) may be covalently attached to the remainder of the molecule through a linker, L^{11A}, L^{10B}, L^{20A}, or L^{20B}. For the complexes and peptide compounds provided herein the therapeutic moiety (e.g., a nanobody, affibody) may be attached to the C-terminus of the peptide compound, to the N-terminus of the peptide compound or a portion of the peptide compound between (connecting) the C-terminus and the N-terminus. In embodiments, the therapeutic moiety is attached to the C-terminus of the peptide compound. In embodiments, the therapeutic moiety is attached to the N-terminus of the peptide compound. Thus, in embodiments, R¹⁰ is a therapeutic moiety. In embodiments, R²⁰ is a therapeutic moiety. In embodiments, the linker L^{10A} L^{10B} L^{20A}, or L^{20B} is independently a peptidyl linker. In embodiments, the linker L^{10A}, L^{10B}, L^{20A}, or L^{20B} is at least 2 amino acids in length. In embodiments, the linker L^{10A}, L^{10B}, L^{20A}, or L^{20B} is at least 4 amino acids in length. In embodiments, the linker L^{10A}, L^{10B}, L^{20A}, or L^{20B} is about 2 amino acids in length. In embodiments, the linker L^{10A}, L^{10B}, L^{20A}, or L^{20B} is about 4 amino acids in length. In embodiments, the linker L^{10A}, L^{10B}, L^{20A}, or L^{20B} is 2 amino acids in length. In embodiments, the linker L^{11A}, L^{10B}, L^{20A} or L^{20B} is 4 amino acids in length. In embodiments, R¹⁰ is a therapeutic moiety and R² is null. In embodiments, R²⁰ is a therapeutic moiety and R¹ is null.

In embodiments, the linker L^{11A}, L^{10B}, L^{20A}, or L^{20B} is from about 2 to about 10 amino acids in length. In embodiments, the linker L^{10A} L^{10B} L^{20A}, or L^{20B} is from about 3 to about 10 amino acids in length. In embodiments, the linker L^{10A}, L^{10B}, L^{20A}, or L^{20B} is from about 4 to about amino acids in length. In embodiments, the linker L^{11A}, L^{10B}, L^{20A}, or L^{20B} is from about 5 to about 10 amino acids in length. In embodiments, the linker L^{11A}, L^{10B}, L^{20A}, or L^{20B} is from about 6 to about 10 amino acids in length. In embodiments, the linker L^{10A}, L^{10B}, L^{20A}, or L^{20B} is from about 7 to about 10 amino acids in length. In embodiments, the linker L^{10A}, L^{10B}, L^{20A}, or L^{20B} is from about 8 to about 10 amino acids in length.

In embodiments, the linker L^{10A}, L^{10B}, L^{20A}, or L^{20B} is from about 2 to about 9 amino acids in length. In embodiments, the linker L^{10A}, L^{10B}, L^{20A}, or L^{20B} is from about 3 to about 9 amino acids in length. In embodiments, the linker L^{10A}, L^{11B}, L^{20A}, or L^{20B} is from about 4 to about 9 amino acids in length. In embodiments, the linker L^{10A}, L^{10B}, L^{20A}, or L^{20B} is from about 5 to about 9 amino acids in length. In embodiments, the linker L^{10A}, L^{10B}, L^{20A}, or L^{20B} is from about 6 to about 9 amino acids in length. In embodiments, the linker L^{10A}, L^{11B}, L^{20A}, or L^{20B} is from about 7 to about 9 amino acids in length.

In embodiments, the linker L^{10A}, L^{10B}, L^{20A}, or L^{20B} is from about 2 to about 8 amino acids in length. In embodiments, the linker L^{10A}, L^{10B}, L^{20A}, or L^{20B} is from about 3 to about 8 amino acids in length. In embodiments, the linker L^{10A}, L^{11B}, L^{20A}, or L^{20B} is from about 4 to about 8 amino acids in length. In embodiments, the linker L^{10A}, L^{10B}, L^{20A}, or L^{20B} is from about 5 to about 8 amino acids in length. In embodiments, the linker L^{10A}, L^{10B}, L^{20A}, or L^{20B} is from about 6 to about 8 amino acids in length. In embodiments, the linker L^{10A}, L^{11B}, L^{20A}, or L^{20B} is from about 2 to about 7 amino acids in length. In embodiments, the linker L^{10A}, L^{11B}, L^{20A}, or L^{20B} is from about 3 to about 7 amino acids in length. In embodiments, the linker L^{10A}, L^{11B}, L^{20A}, or L^{20B} is from about 4 to about 7 amino acids in length. In embodiments, the linker L^{10A}, L^{10B}, L^{20A}, or L^{20B} is from about 5 to about 7 amino acids in length. In embodiments, the linker L^{10A}, L^{10B}, L^{20A}, or L^{20B} is from about 2 to about 6 amino acids in length. In embodiments, the linker L^{10A}, L^{11B}, L^{20A} or L^{20B} is from about 3 to about 6 amino acids in length. In embodiments, the linker L^{10A}, L^{11B}, L^{20A} or L^{20B} is from about 4 to about 6 amino acids in length. In embodiments, the linker L^{10A}, L^{11B}, L^{20A}, or L^{20B} is from about 2 to about 5 amino acids in length. In embodiments, the linker L^{10A}, L^{10B}, L^{20A}, or L^{20B} is from about 3 to about 5 amino acids in length. In embodiments, the linker L^{10A}, L^{10B} L^{20A}, or L^{20B} is from about 2 to about 4 amino acids in length.

In embodiments, the therapeutic moiety is a nanobody moiety and the nanobody moiety is attached to the C-terminus of the peptide compound. Thus, in embodiments, R²⁰ is an anti-CD16 nanobody moiety and L^{20A} or L^{20B} is from about 2 to about 10 amino acids in length. In embodiments, R²⁰ is an anti-CD16 nanobody moiety and L^{20A} or L^{20B} is from about 4 to about 6 amino acids in length. In embodiments, R²⁰ is an anti-CD16 nanobody moiety and L^{20A} or L^{20B} is 4 amino acids in length. In embodiments, R²⁰ is an anti-CD16 nanobody moiety and L^{20A} or L^{20B} is 5 amino acids in length. In embodiments, R²⁰ is an anti-CD16 nanobody moiety and L^{20A} or L^{20B} is 6 amino acids in length. In embodiments, R²⁰ is an anti-CD16 nanobody moiety and L^{20A} or L^{20B} is 7 amino acids in length. In embodiments, R²⁰ is an anti-CD16 nanobody moiety and L^{20A} or L^{20B} is 8 amino acids in length. In embodiments, R²⁰ is an anti-CD16 nanobody moiety and L^{20A} or L^{20B} is 9 amino acids in length. In embodiments, R²⁰ is an anti-CD16 nanobody moiety and L^{20A} or L^{20B} is 10 amino acids in length.

In embodiments, the therapeutic moiety is a nanobody moiety and the nanobody moiety is attached to the N-terminus of the peptide compound. Thus, in embodiments, R¹⁰ is an anti-CD16 nanobody moiety and L^{10A} or L^{10B} are independently from about 2 to about 10 amino acids in length. In embodiments, R¹⁰ is an anti-CD16 nanobody moiety and L^{10A} or L^{10B} are independently from about 4 to about 6 amino acids in length. In embodiments, R¹⁰ is an anti-CD16 nanobody moiety and L^{10A} or L^{10B} are independently 4 amino acids in length. In embodiments, R¹⁰ is an anti-CD16 nanobody moiety and L^{10A} or L^{10B} are independently 5 amino acids in length. In embodiments, R¹⁰ is an anti-CD16 nanobody moiety and L^{10A} or L^{10B} are independently 6 amino acids in length. In embodiments, R¹⁰ is an anti-CD16 nanobody moiety and L^{10A} or L^{10B} are independently 7 amino acids in length. In embodiments, R¹⁰ is an anti-CD16 nanobody moiety and L^{10A} or L^{10B} are independently 8 amino acids in length. In embodiments, R¹⁰ is an anti-CD16 nanobody moiety and L^{10A} or L^{10B} are independently 9 amino acids in length. In embodiments, R¹⁰ is an anti-CD16 nanobody moiety and L^{10A} or L^{10B} are independently 10 amino acids in length.

For the peptide compounds provided herein including embodiments thereof, R²⁰ may be a therapeutic moiety. In embodiments, R²⁰ is a protein moiety. In embodiments, R²⁰ is a nanobody moiety. In embodiments, R²⁰ is a variable heavy chain nanobody moiety. In embodiments, R²⁰ is an anti-CD16 nanobody moiety. In embodiments, L^{20A} or L^{20B} is independently a peptidyl linker. In embodiments, L^{20A} is a peptidyl linker. In embodiments, L^{20B} is a peptidyl linker. In embodiments, L^{20A} or L^{20B} is independently from about 2 to about 10 amino acids in length. In embodiments, L^{20A} or L^{20B} is independently from about 4 to about 6 amino acids in length. In embodiments, L^{20A} is from about 2 to about 10 amino acids in length. In embodiments, L^{20A} is from about 4 to about 6 amino acids in length. In embodiments, L^{20B} is from about 2 to about 10 amino acids in length. In embodiments, L^{20B} is from about 4 to about 6 amino acids in length.

In embodiments, the therapeutic moiety is an affibody (a single chain antigen binding polypeptide) moiety and the affibody moiety is attached to the N-terminus of the peptide compound. Thus, in embodiments, R¹⁰ is an anti-HER2 affibody moiety (also referred to herein as zHER2 moiety) and L^{10A} or L^{10B} are independently from about 2 to about 10 amino acids in length. In embodiments, R¹⁰ is an a anti-HER2 affibody moiety and L^{10A} or L^{10B} are independently from about 4 to about 6 amino acids in length. In embodiments, R¹⁰ is an anti-HER2 affibody moiety and L^{10A} or L^{10B} are independently 4 amino acids in length. In embodiments, R¹⁰ is an anti-HER2 affibody moiety and L^{10A} or L^{10B} are independently 5 amino acids in length. In embodiments, R¹⁰ is an anti-HER2 affibody moiety and L^{10A} or L^{10B} are independently 6 amino acids in length. In embodiments, R¹⁰ is an anti-HER2 affibody moiety and L^{10A} or L^{10B} are independently 7 amino acids in length. In embodiments, R¹⁰ is an anti-HER2 affibody moiety and L^{10A} or L^{10B} are independently 8 amino acids in length. In embodiments, R¹⁰ is an anti-HER2 affibody moiety and L^{10A} or L^{10B} are independently 9 amino acids in length. In embodiments, R¹⁰ is an anti-HER2 affibody moiety and L^{10A} or L^{10B} are independently 10 amino acids in length.

For the peptide compounds provided herein including embodiments thereof, R²⁰ may be a therapeutic moiety. In embodiments, R²⁰ is a protein moiety. In embodiments, R²⁰ is an affibody moiety. In embodiments, R²⁰ is an anti-HER2 affibody moiety. In embodiments, L^{20A} or L^{20B} is independently a peptidyl linker. In embodiments, L^{20A} is a peptidyl linker. In embodiments, L^{20B} is a peptidyl linker. In embodiments, L^{20A} or L^{20B} is independently from about 2 to about 10 amino acids in length. In embodiments, L^{20A} or L^{20B} is independently from about 4 to about 6 amino acids in length. In embodiments, L^{20A} is from about 2 to about 10 amino acids in length. In embodiments, L^{20A} is from about 4 to about 6 amino acids in length. In embodiments, L^{20B} is from about 2 to about 10 amino acids in length. In embodiments, L^{20B} is from about 4 to about 6 amino acids in length.

In embodiments, the peptide compounds of formula (I), (IA), (IB), (II) and (IIA) include a therapeutic moiety (also referred to herein as R¹⁰ or R²⁰) covalently attached to the remainder of the molecule through a linker, L^{10A}, L^{10B}, L^{20A}, or L^{20B}. In embodiments, R¹⁰ and R²⁰ are independently a therapeutic moiety. The term “therapeutic moiety” as provided herein is used in accordance with its plain ordinary meaning and refers to a monovalent compound having a therapeutic benefit (e.g., prevention, eradication, amelioration of the underlying disorder being treated) when given to a subject in need thereof. Therapeutic moieties as provided herein may include, without limitation, peptides, proteins, nucleic acids, nucleic acid analogs, small molecules, antibodies, nanobodies, enzymes, prodrugs, cytotoxic agents (e.g. toxins) including, but not limited to ricin, doxorubicin, daunorubicin, taxol, ethidium bromide, mitomycin, etoposide, tenoposide, vincristine, vinblastine, colchicine, dihydroxy anthracin dione, actinomycin D, diphtheria toxin, Pseudomonas exotoxin (PE) A, PE40, abrin, and glucocorticoid. In embodiments, the therapeutic moiety is an anti-cancer agent or chemotherapeutic agent as described herein. In embodiments, the therapeutic moiety is a nucleic acid moiety, a peptide moiety or a small molecule drug moiety. In embodiments, the therapeutic moiety is a nucleic acid moiety. In embodiments, the therapeutic moiety is an antibody moiety. In embodiments, the therapeutic moiety is a peptide moiety. In embodiments, the therapeutic moiety is a small molecule drug moiety. In embodiments, the therapeutic moiety is a nuclease. In embodiments, the therapeutic moiety is an immunostimulator. In embodiments, the therapeutic moiety is a toxin. In embodiments, the therapeutic moiety is a nuclease. In embodiments, the therapeutic moiety is auristatin. In embodiments, the therapeutic moiety is mertansine.

The peptide compounds of formula (I), (IA), (IB), (II) and (IIA) may include an imaging or detectable moiety. In embodiments, R¹⁰ and R²⁰ are independently a detectable moiety. An “imaging or detectable moiety” as provided herein is a monovalent compound detectable by spectroscopic, photochemical, biochemical, immunochemical, chemical, or other physical means. In embodiments, the imaging moiety is covalently attached to peptide compound. Exemplary imaging moieties are without limitation ³²P, radionuclides, positron-emitting isotopes, fluorescent dyes, fluorophores, antibodies, bioluminescent molecules, chemiluminescent molecules, photoactive molecules, metals, electron-dense reagents, enzymes (e.g., as commonly used in an ELISA), magnetic contrast agents, quantum dots, nanoparticles, biotin, digoxigenin, haptens and proteins or other entities which can be made detectable, e.g., by incorporating a radiolabel into a peptide or antibody specifically reactive with a target peptide. Any method known in the art for conjugating an antibody to the moiety may be employed, e.g., using methods described in Hermanson, Bioconjugate Techniques 1996, Academic Press, Inc., San Diego. Exemplary fluorophores include fluorescein, rhodamine, GFP, coumarin, FITC, ALEXA fluor, Cy3, Cy5, BODIPY, and cyanine dyes. Exemplary radionuclides include Fluorine-18, Gallium-68, and Copper-64. Exemplary magnetic contrast agents include gadolinium, iron oxide and iron platinum, and manganese. In embodiments, the imaging moiety is a bioluminescent molecule. In embodiments, the imaging moiety is a photoactive molecule. In embodiments, the imaging moiety is a metal. In embodiments, the imaging moiety is a nanoparticle.

In embodiments of formula (I), (IA), (IB), (II) or (IIA), X0 is Ser. In embodiments, X0 is null. In embodiments, X1 is Ser. In embodiments, X1 is Cys. In embodiments, X1 is Gly. In embodiments, X1 is β-alanine. In embodiments, X1 is diaminopropionic acid. In embodiments, X1 is β-azidoalanine. In embodiments, X1 is null.

In embodiments, X2 is Gln. In embodiments, X2 is null.

In embodiments, X3 is Phe. In embodiments, X3 is Tyr. In embodiments, X3 is β,β′-diphenyl-Ala. In embodiments, X3 is His. In embodiments, X3 is Asp. In embodiments, X3 is 2-bromo-L-phenylalanine. In embodiments, X3 is 3-bromo-L-phenylalanine. In embodiments, X3 is 4-bromo-L-phenylalanine. In embodiments, X3 is Asn. In embodiments, X3 is Gln. In embodiments, X3 is a modified Phe. In embodiments, X3 is a hydratable carbonyl-containing residue. In embodiments, X3 is a boronic acid-containing residue.

In embodiments, X4 is Asp. In embodiments, X4 is Asn.

In embodiments, X5 is Leu. In embodiments, X5 is β,β′-diphenyl-Ala. In embodiments, X5 is Phe. In embodiments, X5 is Trp. In embodiments, X5 is Tyr. In embodiments, X5 is a non-natural analog of phenylalanine. In embodiments, X5 is tryptophan. In embodiments, X5 is tyrosine. In embodiments, X5 is a hydratable carbonyl-containing residue. In embodiments, X5 is a boronic acid-containing residue.

In embodiments, X6 is Cys. In embodiments, X6 is protected Cys. In embodiments, X6 is Ser. In embodiments, X6 is the thiol side chain amino acid.

In embodiments, X7 is Cys. In embodiments, X7 is protected Cys. In embodiments, X7 is the thiol side chain amino acid. In embodiments, X7 is Thr. In embodiments, X7 is Ser.

In embodiments, X8 is protected Arg. In embodiments, X8 is the thiol side chain amino acid. In embodiments, X8 is Arg. In embodiments, X8 is Ala. In embodiments, X8 is an amino acid comprising a side chain of the formula -L^{3A}-L^{3B}-R³, wherein L^{3A} is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, L^{3B} is a chemical linker and R³ is a steric hindering chemical moiety.

In embodiments, X9 is Cys. In embodiments, X9 is protected Cys. In embodiments, X9 is the thiol side chain amino acid. In embodiments, X9 is Arg. In embodiments, X9 is Ala.

In embodiments, X10 is Leu. In embodiments, X10 is Gln. In embodiments, X10 is Glu. In embodiments, X10 is β,β′-diphenyl-Ala. In embodiments, X10 is Phe. In embodiments, X10 is Trp. In embodiments, X10 is Tyr. In embodiments, X10 is a non-natural analog of phenylalanine. In embodiments, X10 is tryptophan. In embodiments, X10 is tyrosine. In embodiments, X10 is a hydratable carbonyl-containing residue. In embodiments, X10 is a boronic acid-containing residue.

In embodiments, X11 is Cys. In embodiments, X11 is protected Cys. In embodiments, X11 is the thiol side chain amino acid. In embodiments, X11 is Gln. In embodiments, X11 is Lys.

In embodiments, X11 is Arg.

In embodiments, X12 is Ser. In embodiments, X12 is Cys. In embodiments, X12 is protected Cys. In embodiments, X12 is the thiol side chain amino acid. In embodiments, X12 is Gly. In embodiments, X12 is 7-aminoheptanoic acid. In embodiments, X12 is β-alanine. In embodiments, X12 is diaminopropionic acid. In embodiments, X12 is propargylglycine. In embodiments, X12 is isoaspartic acid. In embodiments, X12 is null.

In embodiments of formula (II), X13 is Gly. In embodiments, X13 is Ser.

In embodiments, X14 is Gly. In embodiments, X14 is Ser. In embodiments, X14 is Ala. In embodiments, X14 is Cys. In embodiments, X14 is protected Cys. In embodiments, X14 is the thiol side chain amino acid.

In embodiments, X15 is Gly. In embodiments, X15 is Ser. In embodiments, X15 is Ala. In embodiments, X15 is Cys. In embodiments, X15 is protected Cys. In embodiments, X15 is the thiol side chain amino acid.

In embodiments, X13 and X14 are independently Gly, Ala, Pro, Gln, Asn, Lys, Arg, Glu, Asp, or His.

In embodiments, X6 is the thiol side chain amino acid. In a further embodiment, the thiol side chain amino acid is cysteine. In embodiments, X7 is the thiol side chain amino acid. In a further embodiment, the thiol side chain amino acid is cysteine. In embodiments, X8 is the thiol side chain amino acid. In a further embodiment, the thiol side chain amino acid is substituted arginine. In embodiments, X9 is the thiol side chain amino acid. In a further embodiment, the thiol side chain amino acid is cysteine. In embodiments, X11 is the thiol side chain amino acid. In a further embodiment, the thiol side chain amino acid is cysteine. In embodiments, X11 is the thiol side chain amino acid. In a further embodiment, the thiol side chain amino acid is cysteine.

In embodiments, the first cysteine is at a position corresponding to Kabat position 175 and X6 is the thiol side chain amino acid. In further embodiments, the thiol side chain amino acid is cysteine. In embodiments, the first cysteine is at a position corresponding to Kabat position 174 and X6 is the thiol side chain amino acid. In further embodiments, the thiol side chain amino acid is cysteine.

In embodiments, the first cysteine is at a position corresponding to Kabat position 158 and X8 is the thiol side chain amino acid. In further embodiments, the thiol side chain amino acid is a substituted arginine. In embodiments, the first cysteine is at a position corresponding to Kabat position 208 and X8 is the thiol side chain amino acid. In further embodiments, the thiol side chain amino acid is a substituted arginine.

In embodiments, the first cysteine is at a position corresponding to Kabat position 142 and X15 is the thiol side chain amino acid. In further embodiments, the thiol side chain amino acid is cysteine. In embodiments, the first cysteine is at a position corresponding to Kabat position 143 and X15 is the thiol side chain amino acid. In further embodiments, the thiol side chain amino acid is cysteine.

In embodiments, the first cysteine is at a position corresponding to Kabat position 175 and the peptide compound includes the sequence of SEQ ID NO:1. In embodiments, the first cysteine is at a position corresponding to Kabat position 175 and the peptide compound includes the sequence of SEQ ID NO:4. In embodiments, the first cysteine is at a position corresponding to Kabat position 175 and the peptide compound includes the sequence of SEQ ID NO:22. In embodiments, the first cysteine is at a position corresponding to Kabat position 175 and the peptide compound includes the sequence of SEQ ID NO:27. In embodiments, the first cysteine is at a position corresponding to Kabat position 175 and the peptide compound includes the sequence of SEQ ID NO:23. In embodiments, the first cysteine is at a position corresponding to Kabat position 175 and the peptide compound includes the sequence of SEQ ID NO:28. In embodiments, the first cysteine is at a position corresponding to Kabat position 175 and the peptide compound includes the sequence of SEQ ID NO:44. In embodiments, the first cysteine is at a position corresponding to Kabat position 174 and the peptide compound includes the sequence of SEQ ID NO:1. In embodiments, the first cysteine is at a position corresponding to Kabat position 174 and the peptide compound includes the sequence of SEQ ID NO:4. In embodiments, the first cysteine is at a position corresponding to Kabat position 175 and the peptide compound is the sequence of SEQ ID NO:1. In embodiments, the first cysteine is at a position corresponding to Kabat position 175 and the peptide compound is the sequence of SEQ ID NO:4. In embodiments, the first cysteine is at a position corresponding to Kabat position 174 and the peptide compound is the sequence of SEQ ID NO:1. In embodiments, the first cysteine is at a position corresponding to Kabat position 174 and the peptide compound is the sequence of SEQ ID NO:4.

In embodiments, the first cysteine is at a position corresponding to Kabat position 158 and the peptide compound includes the sequence of SEQ ID NO:2. In embodiments, the first cysteine is at a position corresponding to Kabat position 208 and the peptide compound includes the sequence of SEQ ID NO:2. In embodiments, the first cysteine is at a position corresponding to Kabat position 158 and the peptide compound is the sequence of SEQ ID NO:2. In embodiments, the first cysteine is at a position corresponding to Kabat position 208 and the peptide compound is the sequence of SEQ ID NO:2.

In embodiments, the first cysteine is at a position corresponding to Kabat position 142 and the peptide compound includes the sequence of SEQ ID NO:3. In embodiments, the first cysteine is at a position corresponding to Kabat position 143 and the peptide compound includes the sequence of SEQ ID NO:3. In embodiments, the first cysteine is at a position corresponding to Kabat position 142 and the peptide compound is the sequence of SEQ ID NO:3. In embodiments, the first cysteine is at a position corresponding to Kabat position 143 and the peptide compound is the sequence of SEQ ID NO:3.

In embodiments of formula (I), the thiol side chain amino acid at position X6 is Cys. In embodiments, X8 is Arg. In embodiments, X0 is null. In embodiments, X1 and X12 are independently Ser. In embodiments, X1 and X12 are Ser. In embodiments, X5 is β,β′-diphenyl-Ala. In embodiments, R² is a 1 to 100 amino acid peptide sequence. In embodiments, R¹ is null and R² is a 1 to 100 amino acid peptide sequence. In embodiments, R² is -Gly-Gly-Lys. In embodiments, X1 and X12 are optionally joined together to form a cyclic peptidyl moiety. In embodiments, the peptide compound is a linear peptide compound. A “linear peptide compound” as provided herein is a peptide compound including a linear peptidyl moiety. A linear peptide compound as provided herein does not include a cyclic peptidyl moiety. In embodiments, the peptide compound includes the sequence of SEQ ID NO:1. In embodiments, the peptide compound includes the sequence of SEQ ID NO:22, SEQ ID NO:23, SEQ ID NO:27, SEQ ID NO:28, or SEQ ID NO:44.

In embodiments of formula (I), X6 is Ser. In embodiments, X5 is β,β′-diphenyl-Ala. In embodiments, the thiol side chain amino acid at position X8 is a substituted arginine. In embodiments, the substituted arginine is an octyl-thiol-substituted arginine. In embodiments, X0 and X1 are null. In embodiments, X11 is lysine. In embodiments, R² is a 1 to 100 amino acid peptide sequence. In embodiments, R¹ is null and R² is a 1 to 100 amino acid peptide sequence. In embodiments, R² is -Gly-Gly-Lys. In embodiments, R¹ and X11 are optionally joined together to form a cyclic peptidyl moiety. In embodiments, the peptide compound includes the sequence of SEQ ID NO:2.

In embodiments of formula (I), X6 is Ser. In embodiments, X5 is β,β′-diphenyl-Ala. In embodiments, X8 is Arg. In embodiments, X12 is Ser. In embodiments, R² is a 1 to 100 amino acid peptide sequence. In embodiments, R² is -Ser-Gly-X15-Gly-Lys, wherein X15 is the thiol side chain amino acid. In embodiments, X15 is Cys. In embodiments, X1 and X12 are optionally joined together to form a cyclic peptidyl moiety. In embodiments, the peptide compound includes the sequence of SEQ ID NO:3.

In embodiments of formula (I), the thiol side chain amino acid at position X6 is Cys. In embodiments, X8 is Arg. In embodiments, X0 is null. In embodiments, X1 and X12 are independently Ser. In embodiments, X1 and X12 are Ser. In embodiments, X5 is Phe. In embodiments, R² is a 1 to 100 amino acid peptide sequence. In embodiments, R¹ is null or —C(O)—CH₃ and R² is a 1 to 100 amino acid peptide sequence. In embodiments, R² is -Gly-Gly-Ser-Lys. In embodiments, X1 and X12 are optionally joined together to form a cyclic peptidyl moiety. In embodiments, the peptide compound is a linear peptide compound. In embodiments, the peptide compound includes the sequence of SEQ ID NO:22 or SEQ ID NO:27.

In embodiments of formula (I), the thiol side chain amino acid at position X6 is Cys. In embodiments, X8 is Arg. In embodiments, X0 is null. In embodiments, X1 and X12 are independently Ser. In embodiments, X1 and X12 are Ser. In embodiments, X5 is β,β′-diphenyl-Ala. In embodiments, R² is a 1 to 100 amino acid peptide sequence. In embodiments, R¹ is null or —C(O)—CH₃ and R² is a 1 to 100 amino acid peptide sequence. In embodiments, R² is -Gly-Gly-Ser-Lys. In embodiments, X1 and X12 are optionally joined together to form a cyclic peptidyl moiety.

In embodiments, the peptide compound is a linear peptide compound. In embodiments, the peptide compound includes the sequence of SEQ ID NO:23 or SEQ ID NO:28.

In embodiments of formula (I), X1 is Cys. In embodiments, X8 is Arg. In embodiments, X0 is null. In embodiments, X1 is null. In embodiments, X12 is Cys. In embodiments, X5 is Leu. In embodiments, R¹ is —C(O)—CH₃. In embodiments, the peptide compound is a linear peptide compound. In embodiments, the peptide compound includes the sequence of SEQ ID NO:44.

In embodiments of formula (I), (IA), (IB), (II) or (IIA), R² is a 1 to 100 amino acid peptide sequence. In embodiments, R² is a 1 to 90 amino acid peptide sequence. In embodiments, R² is a 1 to 80 amino acid peptide sequence. In embodiments, R² is a 1 to 70 amino acid peptide sequence. In embodiments, R² is a 1 to 60 amino acid peptide sequence. In embodiments, R² is a 1 to 50 amino acid peptide sequence. In embodiments, R² is a 1 to 40 amino acid peptide sequence. In embodiments, R² is a 1 to 30 amino acid peptide sequence. In embodiments, R² is a 1 to 20 amino acid peptide sequence. In embodiments, R² is a 1 to 10 amino acid peptide sequence. In embodiments, R¹ is null and R² is a 1 to 100 amino acid peptide sequence. In embodiments, R¹ is null and R² is a 1 to 90 amino acid peptide sequence. In embodiments, R¹ is null and R² is a 1 to 80 amino acid peptide sequence. In embodiments, R¹ is null and R² is a 1 to 70 amino acid peptide sequence. In embodiments, R¹ is null and R² is a 1 to 60 amino acid peptide sequence. In embodiments, R¹ is null and R² is a 1 to 50 amino acid peptide sequence. In embodiments, R¹ is null and R² is a 1 to 40 amino acid peptide sequence. In embodiments, R¹ is null and R² is a 1 to 30 amino acid peptide sequence. In embodiments, R¹ is null and R² is a 1 to 20 amino acid peptide sequence. In embodiments, R¹ is null and R² is a 1 to 10 amino acid peptide sequence. In embodiments of formula (I), (IA), (IB), (II) or (IIA), R¹ is a 1 to 100 amino acid peptide sequence. In embodiments, R¹ is a 1 to 90 amino acid peptide sequence. In embodiments, R¹ is a 1 to 80 amino acid peptide sequence. In embodiments, R¹ is a 1 to 70 amino acid peptide sequence. In embodiments, R¹ is a 1 to 60 amino acid peptide sequence. In embodiments, R¹ is a 1 to 50 amino acid peptide sequence. In embodiments, R¹ is a 1 to 40 amino acid peptide sequence. In embodiments, R¹ is a 1 to 30 amino acid peptide sequence. In embodiments, R¹ is a 1 to 20 amino acid peptide sequence. In embodiments, R¹ is a 1 to 10 amino acid peptide sequence. In embodiments, R¹ is null and R¹ is a 1 to 100 amino acid peptide sequence. In embodiments, R² is null and R¹ is a 1 to 90 amino acid peptide sequence. In embodiments, R² is null and R¹ is a 1 to 80 amino acid peptide sequence. In embodiments, R² is null and R¹ is a 1 to 70 amino acid peptide sequence. In embodiments, R² is null and R¹ is a 1 to 60 amino acid peptide sequence. In embodiments, R² is null and R¹ is a 1 to 50 amino acid peptide sequence. In embodiments, R² is null and R¹ is a 1 to 40 amino acid peptide sequence. In embodiments, R² is null and R¹ is a 1 to 30 amino acid peptide sequence. In embodiments, R² is null and R¹ is a 1 to 20 amino acid peptide sequence. In embodiments, R² is null and R¹ is a 1 to 10 amino acid peptide sequence.

In embodiments of formula (II), R² is a 1 to 10 amino acid peptide sequence. In embodiments of formula (II), R¹ is null and R² is a 1 to 10 amino acid peptide sequence. In embodiments, R² is -Gly-Lys. In embodiments, the peptide compound includes the sequence of SEQ ID NO:3.

In embodiments, R¹⁰ and R²⁰ are independently substituted (e.g., substituted with a substituent group, a size-limited substituent or a lower substituent group) or unsubstituted alkyl, substituted (e.g., substituted with a substituent group, a size-limited substituent or a lower substituent group) or unsubstituted heteroalkyl, substituted (e.g., substituted with a substituent group, a size-limited substituent or a lower substituent group) or unsubstituted cycloalkyl, substituted (e.g., substituted with a substituent group, a size-limited substituent or a lower substituent group) or unsubstituted heterocycloalkyl, substituted (e.g., substituted with a substituent group, a size-limited substituent or a lower substituent group) or unsubstituted aryl or substituted (e.g., substituted with a substituent group, a size-limited substituent or a lower substituent group) or unsubstituted heteroaryl.

R¹⁰ and R²⁰ may independently be substituted or unsubstituted (e.g., C₁-C₂₀, C₁-C₁₀, C₁-C₅) alkyl, substituted or unsubstituted (e.g., 2 to 20 membered, 2 to 10 membered, 2 to 5 membered) heteroalkyl, substituted or unsubstituted (e.g., C₃-C₅, C₃-C₆, C₃-C₅) cycloalkyl, substituted or unsubstituted (e.g., 3 to 8 membered, 3 to 6 membered, 3 to 5 membered) heterocycloalkyl, substituted or unsubstituted (e.g., C₆-C₁₀, C₆-C₅, C₆-C₅) aryl or substituted or unsubstituted (e.g., 5 to 10 membered, 5 to 8 membered, 5 to 6 membered,) heteroaryl.

R¹⁰ and R²⁰ may independently be unsubstituted (e.g., C₁-C₂₀, C₁-C₁₀, C₁-C₅) alkyl, unsubstituted (e.g., 2 to 20 membered, 2 to 10 membered, 2 to 5 membered) heteroalkyl, unsubstituted (e.g., C₃-C₅, C₃-C₆, C₃-C₅) cycloalkyl, unsubstituted (e.g., 3 to 8 membered, 3 to 6 membered, 3 to 5 membered) heterocycloalkyl, unsubstituted (e.g., C₆-C₁₀, C₆-C₅, C₆-C₅) aryl or unsubstituted (e.g., 5 to 10 membered, 5 to 8 membered, 5 to 6 membered,) heteroaryl.

In embodiments, the antigen binding domain includes a fragment antigen-binding (Fab) domain. In embodiments, the antigen binding domain includes an Fc domain. In embodiments, the antigen binding domain is a fragment antigen-binding (Fab) domain. In embodiments, the antigen binding domain is a humanized antigen binding domain.

In embodiments, the non-CDR peptide binding region is formed by amino acid residues at positions 8, 9, 10, 38, 39, 40, 41, 42, 43, 44, 45, 82, 83, 84, 85, 86, 87, 99, 100, 101, 102, 103, 104, 105, 142, 162, 163, 164, 165, 166, 167, 168, and 173 of the VL region and 6, 9, 38, 39, 40, 41, 42, 43, 44, 45, 84, 86, 87, 88, 89, 90, 91, 103, 104, 105, 106, 107, 108, 111, 110, 147, 150, 151, 152, 173, 174, 175, 176, 177, 185, 186, and 187 of the VH region, according to Kabat numbering.

In embodiments, the non-CDR peptide binding region includes a Glu at position 83 of the VL region, according to Kabat numbering. In embodiments, the non-CDR peptide binding region includes a Thr or Ser at position 40 of the VH region, according to Kabat numbering. In embodiments, the non-CDR peptide binding region includes an Asn at position 41 of the VL region, according to Kabat numbering. In embodiments, the non-CDR peptide binding region includes an Asp or Asn at position 85 of the VL region, according to Kabat numbering. In embodiments, the antigen binding domain binds to an antigen with increased affinity relative to the absence of the peptide compound.

In embodiments, the antigen binding domain binds to an antigen with increased affinity relative to the absence of the peptide compound. Where the antigen binding domain binds to an antigen with increased affinity relative to the absence of the peptide compound, the binding of the antigen binding domain to the antigen is stronger in the presence of the peptide compound than in the absence of the peptide compound.

In embodiments, the antigen binding domain binds to an antigen with a K_{D} of less than 100 nM. In embodiments, the antigen binding domain binds to an antigen with a K_{D} of less than 95 nM. In embodiments, the antigen binding domain binds to an antigen with a K_{D} of less than 90 nM. In embodiments, the antigen binding domain binds to an antigen with a K_{D} of less than 85 nM. In embodiments, the antigen binding domain binds to an antigen with a K_{D} of less than 80 nM. In embodiments, the antigen binding domain binds to an antigen with a K_{D} of less than 75 nM. In embodiments, the antigen binding domain binds to an antigen with a K_{D} of less than 70 nM. In embodiments, the antigen binding domain binds to an antigen with a K_{D} of less than 65 nM. In embodiments, the antigen binding domain binds to an antigen with a K_{D} of less than 60 nM. In embodiments, the antigen binding domain binds to an antigen with a K_{D} of less than 55 nM. In embodiments, the antigen binding domain binds to an antigen with a K_{D} of less than 50 nM. In embodiments, the antigen binding domain binds to an antigen with a K_{D} of less than 45 nM. In embodiments, the antigen binding domain binds to an antigen with a K_{D} of less than 40 nM. In embodiments, the antigen binding domain binds to an antigen with a K_{D} of less than 35 nM. In embodiments, the antigen binding domain binds to an antigen with a K_{D} of less than 30 nM. In embodiments, the antigen binding domain binds to an antigen with a K_{D} of less than 25 nM. In embodiments, the antigen binding domain binds to an antigen with a K_{D} of less than 20 nM. In embodiments, the antigen binding domain binds to an antigen with a K_{D} of less than 15 nM. In embodiments, the antigen binding domain binds to an antigen with a K_{D} of less than 10 nM. In embodiments, the antigen binding domain binds to an antigen with a K_{D} of less than 9 nM. In embodiments, the antigen binding domain binds to an antigen with a K_{D} of less than 8 nM. In embodiments, the antigen binding domain binds to an antigen with a K_{D} of less than 7 nM. In embodiments, the antigen binding domain binds to an antigen with a K_{D} of less than 6 nM. In embodiments, the antigen binding domain binds to an antigen with a K_{D} of less than 5 nM. In embodiments, the antigen binding domain binds to an antigen with a K_{D} of less than 4 nM. In embodiments, the antigen binding domain binds to an antigen with a K_{D} of less than 3 nM. In embodiments, the antigen binding domain binds to an antigen with a K_{D} of less than 2 nM. In embodiments, the antigen binding domain binds to an antigen with a K_{D} of less than 1 nM.

### Peptide Compounds

The peptide compounds provided herein may be linear or cyclic compounds (i.e., compounds including linear or cyclic peptidyl moieties) and may include a steric hindering chemical moiety, a therapeutic or a diagnostic moiety. In embodiments, the peptide compound is cyclized (e.g. cyclized through amino acid side chain moieties). For the peptide compounds described in this section the same definitions and embodiments are applicable as for the peptide compounds defined in the section describing covalent complexes with the exception that the peptide compounds described here are not complexed. Thus, in another aspect, a peptide compound of formula:

R¹-X0-X1-X2-X3-X4-X5-X6-X7-X8-X9-X10-X11-X12-R² (I)

is provide. In formula (I), X0 is Ser or null. X1 is Ser, Cys, Gly, β-alanine, diaminopropionic acid, β-azidoalanine, or null. X2 is Gln or null. X3 is Phe, Tyr, β,β′-diphenyl-Ala, His, Asp, 2-bromo-L-phenylalanine, 3-bromo-L-phenylalanine, 4-bromo-L-phenylalanine, Asn, Gln, a modified Phe, a hydratable carbonyl-containing residue, or a boronic acid-containing residue. X4 is Asp or Asn. X5 is Leu, β,β′-diphenyl-Ala, Phe, Trp, Tyr, a non-natural analog of phenylalanine, tryptophan, or tyrosine, a hydratable carbonyl-containing residue, or a boronic acid-containing residue. X6 is Cys, protected Cys or Ser. X7 is Cys, protected Cys, Thr, or Ser. X8 is protected Arg, Arg, Ala, or an amino acid comprising a side chain of the formula -L^{3A}-L^{3B}-R³, wherein L^{3A} is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, L^{3B} is a chemical linker and R³ is a steric hindering chemical moiety. X9 is Cys, protected Cys, Arg or Ala. X10 is Leu, Gln, Glu, β,β′-diphenyl-Ala, Phe, Trp, Tyr; a non-natural analog of phenylalanine, tryptophan, or tyrosine, a hydratable carbonyl-containing residue, or a boronic acid-containing residue. X11 is Cys, protected Cys, Gln, Lys or Arg. X12 is Ser, Cys, protected Cys, Gly, 7-aminoheptanoic acid, β-alanine, diaminopropionic acid, propargylglycine, isoaspartic acid, or null. R¹ is null, -L^{10A}-L^{10B}-R¹⁰, an amino acid peptide sequence optionally substituted with -L^{10A}-L^{10B}-R¹⁰. R² is null, -L^{20A}-L^{20B}-R²⁰, an amino acid peptide sequence optionally substituted with -L^{20A}-L^{20B}-R²⁰. L^{10A}, L^{10B}, L^{20A}, L^{20B} are independently a bond, a peptidyl linker, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. R¹⁰ and R²⁰ are independently a reactive moiety, a diagnostic moiety, a therapeutic moiety or a detectable moiety. X1 and X12 are optionally joined together to form a cyclic peptidyl moiety. R¹ and X11 are optionally joined together to form a cyclic peptidyl moiety.

A “protected amino acid residue” (e.g., a protected Cys or protected Arg) as provided herein refers to an amino acid which is covalently attached to a protecting or leaving group. The protecting or leaving group may be attached to the side chain of the amino acid. The terms “protecting group” or “leaving group” are used based on their general meaning well known in the chemical arts. Exemplary leaving groups include without limitation any of the amino acid protecting groups described in Isidro-Llobet et al. (Chem. Rev., 2009, 109 (6), pp 2455-2504) and Andreu et al. (Methods in Molecular Biology, Vol. 35, Chapter 7, Peptide Synthesis Protocols, 1994, Humana Press Inc.), which are hereby incorporated in their entirety and for all purposes. In embodiments, the protected Cys includes a thio-pyrimidine moiety. In embodiments, the protected Cys includes a thio-pyridine moiety. In embodiments, the thio-pyridine moiety is covalently attached (through a disulfide bond) to the side chain of the Cys. Thus, in embodiments, the protected Cys is a thio-pyridine-substituted Cys. In embodiments, the thio-pyridine moiety has the formula:

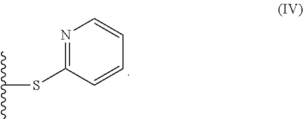 [SMILES: *Sc1ccccn1]

In formula (IV), [symbol] denotes the point of attachment to the amino acid side chain.

In embodiments of formula (I), X6 is Cys. In embodiments of formula (I), X6 is protected Cys. In embodiments, X8 is Arg. In embodiments, X0 is null. In embodiments, X1 and X12 are independently Ser. In embodiments, X1 and X12 are Ser. In embodiments, X5 is β,β′-diphenyl-Ala. In embodiments, R² is a 1 to 100 amino acid peptide sequence. In embodiments, R¹ is null and R² is a 1 to 100 amino acid peptide sequence. In embodiments, R² is -Gly-Gly-Lys. In embodiments, X1 and X12 are optionally joined together to form a cyclic peptidyl moiety. In embodiments, the peptide compound includes the sequence of SEQ ID NO:1.

In embodiments of formula (I), X6 is Ser. In embodiments, X5 is β,β′-diphenyl-Ala. In embodiments, X8 is Arg. In embodiments, X12 is Ser. In embodiments, R² is a 1 to 100 amino acid peptide sequence. In embodiments, R¹ is null and R² is a 1 to 100 amino acid peptide sequence. In embodiments, R² is -Ser-Gly-X15-Gly-Lys, wherein X15 is Cys or protected Cys. In embodiments, X15 is Cys. In embodiments, X15 is protected Cys. In embodiments, X1 and X12 are optionally joined together to form a cyclic peptidyl moiety. In embodiments, the peptide compound includes the sequence of SEQ ID NO:3.

In embodiments of formula (I), the thiol side chain amino acid at position X6 is Cys. In embodiments, X8 is Arg. In embodiments, X0 is null. In embodiments, X1 and X12 are independently Ser. In embodiments, X1 and X12 are Ser. In embodiments, X5 is Phe. In embodiments, R² is a 1 to 100 amino acid peptide sequence. In embodiments, R¹ is null or —C(O)—CH₃ and R² is a 1 to 100 amino acid peptide sequence. In embodiments, R² is -Gly-Gly-Ser-Lys. In embodiments, X1 and X12 are optionally joined together to form a cyclic peptidyl moiety. In embodiments, the peptide compound is a linear peptide compound. In embodiments, the peptide compound includes the sequence of SEQ ID NO:22 or SEQ ID NO:27.

In embodiments of formula (I), the thiol side chain amino acid at position X6 is Cys. In embodiments, X8 is Arg. In embodiments, X0 is null. In embodiments, X1 and X12 are independently Ser. In embodiments, X1 and X12 are Ser. In embodiments, X5 is β,β′-diphenyl-Ala. In embodiments, R² is a 1 to 100 amino acid peptide sequence. In embodiments, R¹ is null or —C(O)—CH₃ and R² is a 1 to 100 amino acid peptide sequence. In embodiments, R² is -Gly-Gly-Ser-Lys. In embodiments, X1 and X12 are optionally joined together to form a cyclic peptidyl moiety. In embodiments, the peptide compound is a linear peptide compound. In embodiments, the peptide compound includes the sequence of SEQ ID NO:23 or SEQ ID NO:28.

In embodiments of formula (I), X1 is Cys. In embodiments, X8 is Arg. In embodiments, X0 is null. In embodiments, X1 is null. In embodiments, X12 is Cys. In embodiments, X5 is Leu. In embodiments, R¹ is —C(O)—CH₃. In embodiments, the peptide compound is a linear peptide compound. In embodiments, the peptide compound includes the sequence of SEQ ID NO:44.

In embodiments, the peptide compound has the structure

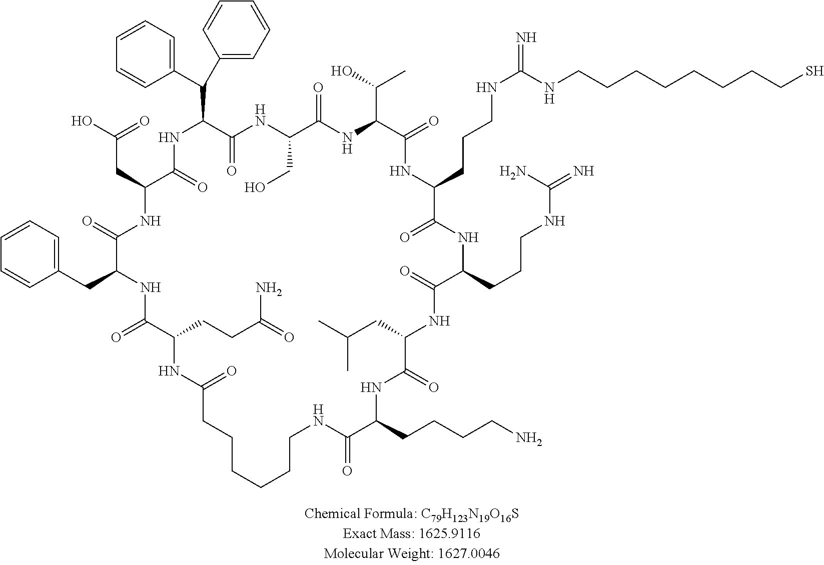 [SMILES: CC(C)C[C@@H]1NC(=O)[C@H](CCCNC(=N)N)NC(=O)[C@H](CCCNC(=N)NCCCCCCCCS)NC(=O)[C@H]([C@@H](C)O)NC(=O)[C@H](CO)NC(=O)[C@H](C(c2ccccc2)c2ccccc2)NC(=O)[C@H](CC(=O)O)NC(=O)[C@H](Cc2ccccc2)NC(=O)[C@H](CCC(N)=O)NC(=O)CCCCCCNC(=O)[C@H](CCCCN)NC1=O]

In another aspect, a peptide compound of formula:

R¹—X0-X1-X2-X3-X4-X5-X6-X7-X8-X9-X10-X11-X12-X13-X14-X15-R² (II)

is provided. In formula (II), X0 is Ser or null. X1 is Ser, Cys, Gly, β-alanine, diaminopropionic acid, β-azidoalanine, or null. X2 is Gln or null. X3 is Phe, Tyr, β,β′-diphenyl-Ala, His, Asp, 2-bromo-L-phenylalanine, 3-bromo-L-phenylalanine, 4-bromo-L-phenylalanine, Asn, Gln, a modified Phe, a hydratable carbonyl-containing residue, or a boronic acid-containing residue. X4 is Asp or Asn. X5 is Leu, β,β′-diphenyl-Ala, Phe, Trp, Tyr, a non-natural analog of phenylalanine, tryptophan, or tyrosine, a hydratable carbonyl-containing residue, or a boronic acid-containing residue. X6 is Ser. X7 is Cys, protected Cys, Thr, or Ser. X8 is Arg, Ala, or an amino acid comprising a side chain of the formula -L^{3A}-L^{3B}-R³, wherein L^{3A} is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, L^{3B} is a chemical linker and R³ is a steric hindering chemical moiety. X9 is Cys, protected Cys, Arg or Ala. X10 is Leu, Gln, Glu, β,β′-diphenyl-Ala, Phe, Trp, Tyr; a non-natural analog of phenylalanine, tryptophan, or tyrosine, a hydratable carbonyl-containing residue, or a boronic acid-containing residue. X11 is Cys, protected Cys, Gln, Lys or Arg. X12 is Ser, Cys, protected Cys, Gly, 7-aminoheptanoic acid, β-alanine, diaminopropionic acid, propargylglycine, isoaspartic acid, or null. X13 is Gly or Ser. X14 and X15 are independently Gly, Ser, Ala, Cys or protected Cys. R¹ is null, -L^{10A}-L^{10B}-R¹⁰, an amino acid peptide sequence optionally substituted with -L^{10A}-L^{10B}-R₁₀. R² is null, -L^{20A}-L^{20B}-R²⁰, an amino acid peptide sequence optionally substituted with -L^{20A}-L^{20B}-R²⁰. L^{10A}, L^{10B}, L^{20A}, L^{20B} are independently a bond, a peptidyl linker, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. R¹⁰ and R²⁰ are independently a reactive moiety, a diagnostic moiety, a therapeutic moiety or a detectable moiety. X1 and X12 are optionally joined together to form a cyclic peptidyl moiety.

In embodiments, X15 is Cys. In embodiments, X15 is protected Cys. In embodiments, R² is a 1 to 100 amino acid peptide sequence. In embodiments, R¹ is null and R² is a 1 to 100 amino acid peptide sequence. In embodiments, R² is -Gly-Lys. In embodiments, the peptide compound includes the sequence of SEQ ID NO:3.

In embodiments, the peptide compound has the sequence of SEQ ID NO:1. In embodiments, the peptide compound has the sequence of SEQ ID NO:4. In embodiments, the peptide compound has the sequence of SEQ ID NO.23. In embodiments, the peptide compound has the sequence of SEQ ID NO.28. In one embodiment, X0 is null, X1 is Ser, X2 is Gln, X3 is Phe, X4 is Asp, X5 is β,β′-diphenyl-Ala, X6 is Cys, X7 is Thr, X8 and X9 are Arg, X10 is Leu, X11 is Gln, X12 is Ser, R¹ is —C(O)CH₃ and R² is -Gly-Gly-Lys or -Gly-Gly-Ser-Lys

In one embodiment, X0 is null, X1 is Ser, X2 is Gln, X3 is Phe, X4 is Asp, X5 is β,β′-diphenyl-Ala, X6 is thio-pyridine-substituted Cys, X7 is Thr, X8 and X9 are Arg, X10 is Leu, X11 is Gln, X12 is Ser, R¹ is —C(O)CH₃ and R² is -Gly-Gly-Lys.

In one embodiment, X0 is null, X1 is Ser, X2 is Gln, X3 is Phe, X4 is Asp, X5 is β,β′-diphenyl-Ala, X6 is Cys, X7 is Thr, X8 and X9 are Arg, X10 is Leu, X11 is Gln, X12 is Ser, R¹ is —C(O)CH₃ and R² is -L^{20A}-L^{20B}-R²⁰, wherein L^{20A} is a peptidyl linker having the sequence -Gly-Gly-Lys-, -L^{20B} is a bond and —R²⁰ is a detectable moiety.

In one embodiment, X0 is null, X1 is Ser, X2 is Gln, X3 is Phe, X4 is Asp, X5 is β,β′-diphenyl-Ala, X6 is thio-pyridine-substituted Cys, X7 is Thr, X8 and X9 are Arg, X10 is Leu, X11 is Gln, X12 is Ser, R¹ is —C(O)CH₃ and R² is -L^{20A}-L^{20B}-R²⁰, wherein L^{20A} is a peptidyl linker having the sequence -Gly-Gly-Lys-, -L^{20B} is a bond and —R²⁰ is a detectable moiety.

In one embodiment, X0 is null, X1 is Ser, X2 is Gln, X3 is Phe, X4 is Asp, X5 is β,β′-diphenyl-Ala, X6 is Cys, X7 is Thr, X8 and X9 are Arg, X10 is Leu, X11 is Gln, X12 is Ser, R¹ is —C(O)CH₃ and R² is -L^{20A}-L^{20B}-R²⁰, wherein L^{20A} is a peptidyl linker having the sequence -Gly-Gly-Lys-, -L^{20B} is a bond and —R²⁰ is a reactive moiety.

In one embodiment, X0 is null, X1 is Ser, X2 is Gln, X3 is Phe, X4 is Asp, X5 is β,β′-diphenyl-Ala, X6 is thio-pyridine-substituted Cys, X7 is Thr, X8 and X9 are Arg, X10 is Leu, X11 is Gln, X12 is Ser, R¹ is —C(O)CH₃ and R² is -L^{20A}-L^{20B}-R²⁰, wherein L^{20A} is a peptidyl linker having the sequence -Gly-Gly-Lys-, -L^{20B} is a bond and —R²⁰ is a reactive moiety.

In embodiments, the peptide compound has the sequence of SEQ ID NO:2. In one embodiment, X0 is null, X1 is null, X2 is Gln, X3 is Phe, X4 is Asp, X5 is β,β′-diphenyl-Ala, X6 is Ser, X7 is Thr, X8 is n-octylthiol-substituted Arg, X9 is Arg, X10 is Leu, X11 is Lys, X12 is Ser, R¹ is aminoheptanoic acid and R²is -Gly-Gly-Lys.

In embodiments, the peptide compound has the sequence of SEQ ID NO: 3. In one embodiment, X0 is null, X1 is Ser, X2 is Gln, X3 is Phe, X4 is Asp, X5 is β,β′-diphenyl-Ala, X6 is Ser, X7 is Thr, X8 and X9 are Arg, X10 is Leu, X11 is Gln, X12 is Ser, R¹ is —C(O)CH₃ and R² is -Ser-Gly-Cys-Gly-Lys.

In embodiments, the peptide compound has the sequence of SEQ ID NO: 3. In one embodiment, X0 is null, X1 is Ser, X2 is Gln, X3 is Phe, X4 is Asp, X5 is β,β′-diphenyl-Ala, X6 is Ser, X7 is Thr, X8 and X9 are Arg, X10 is Leu, X11 is Gln, X12 is Ser, R¹ is —C(O)CH₃ and R² is -Ser-Gly-X15-Gly-Lys, wherein X15 is thio-pyridine-substituted Cys.

In embodiments, the peptide compound has the sequence of SEQ ID NO: 3. In one embodiment, X0 is null, X1 is Ser, X2 is Gln, X3 is Phe, X4 is Asp, X5 is β,β′-diphenyl-Ala, X6 is Ser, X7 is Thr, X8 and X9 are Arg, X10 is Leu, X11 is Gln, X12 and X13 are Ser, X14 is Gly, X15 is Cys, R¹ is —C(O)CH₃ and R² is -Gly-Lys.

In embodiments, the peptide compound has the sequence of SEQ ID NO: 3. In one embodiment, X0 is null, X1 is Ser, X2 is Gln, X3 is Phe, X4 is Asp, X5 is β,β′-diphenyl-Ala, X6 is Ser, X7 is Thr, X8 and X9 are Arg, X10 is Leu, X11 is Gln, X12 and X13 are Ser, X14 is Gly, X15 is thio-pyridine-substituted Cys, R¹ is —C(O)CH₃ and R² is -Gly-Lys.

In embodiments, the peptide compound has the sequence of SEQ ID NO:22. In embodiments, the peptide compound has the sequence of SEQ ID NO:27. In one embodiment, X0 is Ser, X1 is Gln, X2 is Phe, X3 is Asp, X4 is Phe, X5 is Cys, X6 is Thr, X7 is Arg, X8 is Arg, X9 is Leu, X10 is Gln, X11 is Ser, X12 is Gly, X13 is Gly, X14 is Ser, X15 is Lys, R¹ is —C(O)CH₃ or null and R² is null.

In embodiments, the peptide compound has the sequence of SEQ ID NO:24. In embodiments, the peptide compound has the sequence of SEQ ID NO:26. In one embodiment, X0 is Ser, X1 is Gln, X2 is Phe, X3 is Asp, X5 is β,β′-diphenyl-Ala, X6 is Ser, X7 is Thr, X8 and X9 are Arg, X10 is Leu, X11 is Gln, X12 is Ser, X13 is Gly, X14 is Gly, X15 is Ser, R¹ is —C(O)CH₃ and R² is -Lys.

In embodiments, the peptide compound has the sequence of SEQ ID NO:44. In one embodiment, X0 is null, X1 is Cys, X2 is Gln, X3 is Phe, X4 is Asp, X5 is Leu, X6 is Ser, X7 is Thr, X8 and X9 are Arg, X10 is Leu, X11 is Lys, X12 is Cys, R¹ is —C(O)CH₃ and R² null.

In embodiments, the peptide compound has the sequence of SEQ ID NO:25.

### Antigen Binding Domain

For the antigen binding domain compositions described herein the same definitions and embodiments are applicable as for the antigen binding domain defined in the section describing covalent complexes. For example, the antigen binding domain described herein may include a peptide binding site including a cysteine at a position corresponding to Kabat position 175; may include or be a Fab and the non-CDR binding site may include framework region amino acid residues. Thus, in another aspect, an antigen binding domain is provided. The antigen binding domain includes: (1) a central hole enclosed by the heavy chain variable (VH) region, the light chain variable (VL) region, the heavy chain constant (CH1) region and the light chain constant (CL) region of the antigen binding domain between a first cavity and a second cavity; and (2) a non-CDR peptide binding region including: (a) the first cavity lined by a first set of amino acid residues of the VH, VL, CH1, and CL regions of the antigen binding domain, wherein the first set of amino acid residues includes a cysteine at a position corresponding to Kabat position 102, 142 or 143 of the VL region; (b) the second cavity lined by a second set of amino acid residues of the VH, VL, CH1, and CL regions of the antigen binding domain, wherein the second set of amino acid residues includes a cysteine at a position corresponding to Kabat position 208 or 158 of the VH region; or (c) a hole region enclosing the hole between the first cavity and the second cavity, the hole region lined by a third set of amino acid residues of the VH, VL, CH1, and CL regions of the antigen binding domain, wherein the third set of amino acid residues includes a cysteine at a position corresponding to Kabat position 174 or 175 of the VH region.

In embodiments, the first set of amino acid residues includes the first cysteine at a position corresponding to Kabat position 102 of the VL region. In embodiments, the first set of amino acid residues includes the first cysteine at a position corresponding to Kabat position 142 of the VL region. In embodiments, the first set of amino acid residues includes the first cysteine at a position corresponding to Kabat position 143 of the VL region.

In embodiments, the second set of amino acid residues includes the first cysteine at a position corresponding to Kabat position 208 of the VH region. In embodiments, the second set of amino acid residues includes the first cysteine at a position corresponding to Kabat position 158 of the VH region.

In embodiments, the third set of amino acid residues includes the first cysteine at a position corresponding to Kabat position 174 of the VH region. In embodiments, the third set of amino acid residues includes the first cysteine at a position corresponding to Kabat position 175 of the VH region.

In embodiments, the antigen binding domain includes a fragment antigen-binding (Fab) domain. In embodiments, the antigen binding domain includes an Fc domain. In embodiments, the antigen binding domain is a fragment antigen-binding (Fab) domain. In embodiments, the antigen binding domain is a humanized antigen binding domain. In embodiments, the non-CDR peptide binding region includes framework region amino acid residues.

In embodiments, the non-CDR peptide binding region is formed by amino acid residues at positions 8, 9, 10, 38, 39, 40, 41, 42, 43, 44, 45, 82, 83, 84, 85, 86, 87, 99, 100, 101, 102, 103, 104, 105, 142, 162, 163, 164, 165, 166, 167, 168, and 173 of the VL region and 6, 9, 38, 39, 40, 41, 42, 43, 44, 45, 84, 86, 87, 88, 89, 90, 91, 103, 104, 105, 106, 107, 108, 111, 110, 147, 150, 151, 152, 173, 174, 175, 176, 177, 185, 186, and 187 of the VH region, according to Kabat numbering.

In embodiments, the non-CDR peptide binding region includes a Glu at position 83 of the VL region, according to Kabat numbering. In embodiments, the non-CDR peptide binding region includes a Thr or Ser at position 40 of the VH region, according to Kabat numbering. In embodiments, the non-CDR peptide binding region includes an Asn at position 41 of the VL region, according to Kabat numbering. In embodiments, the non-CDR peptide binding region includes an Asp or Asn at position 85 of the VL region, according to Kabat numbering.

### EXAMPLES

### Example 1: Cys-Meditopes

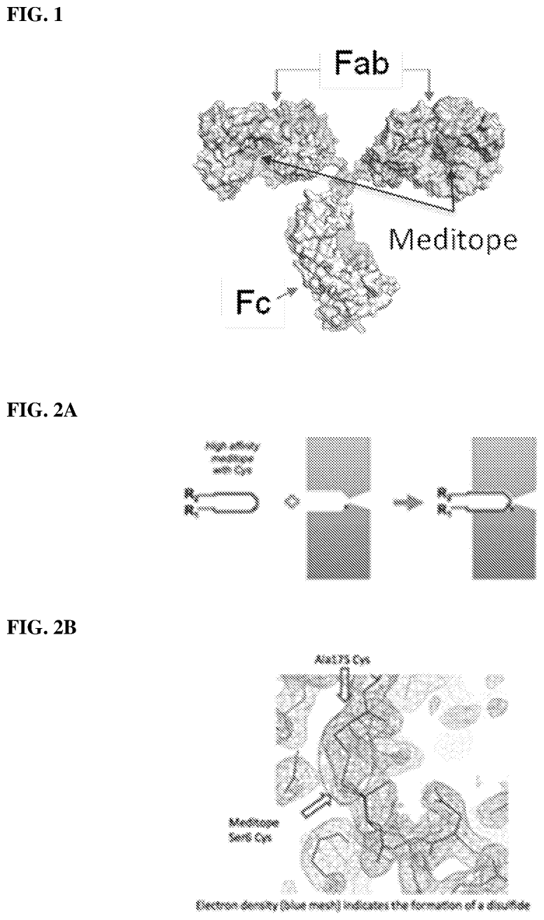

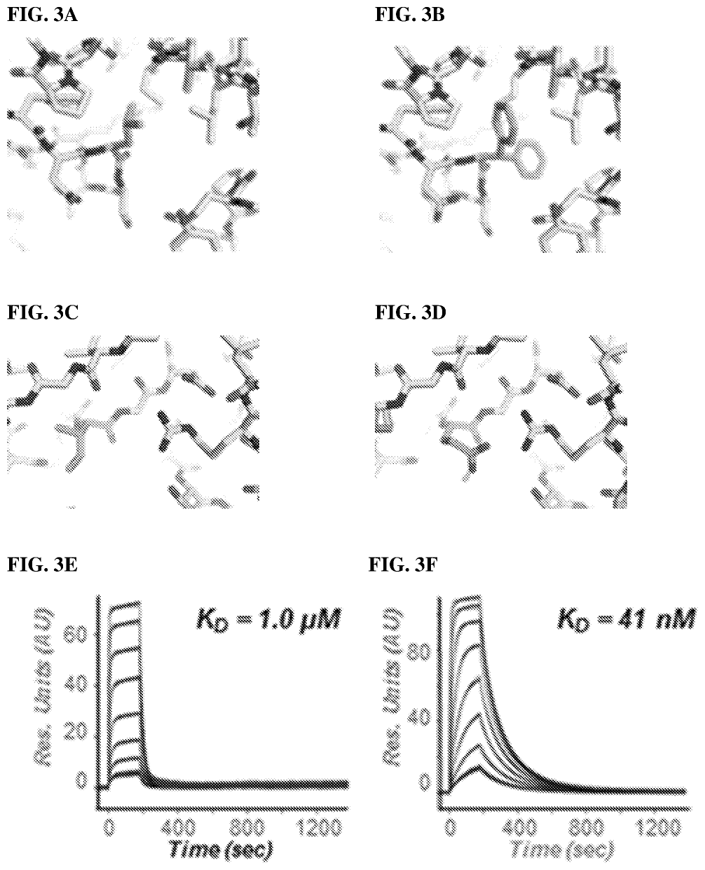

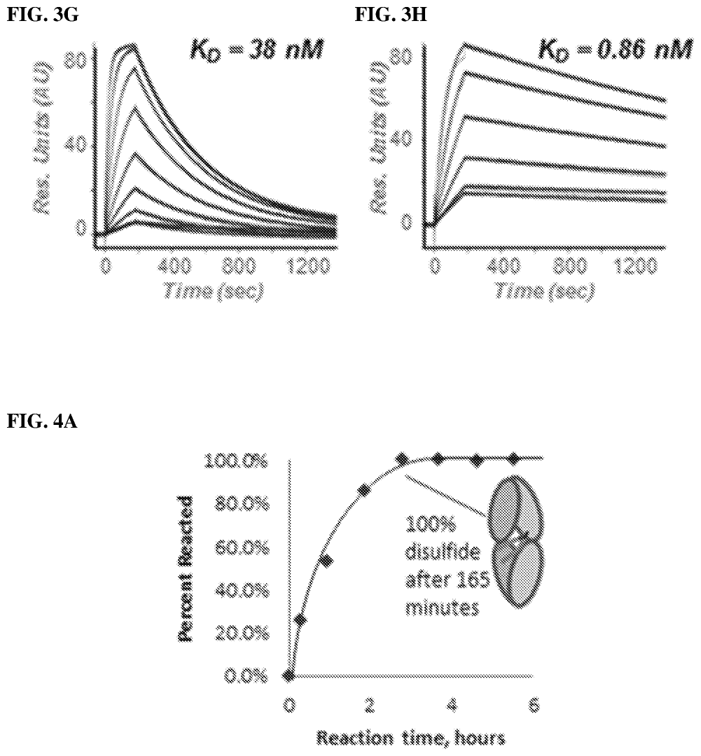

In some instances it is desirable to add functionality to monoclonal antibodies through a covalent bond. To accomplish this, Applicants used the meditope/meditope-enabled Fab (meFab) interaction to create a disulfide bond (FIG. 1 and FIG. 2A).

Based on careful examination of the meditope-meFab interaction and previous observations that cysteine (Cys) introduced at K208 through site directed mutagenesis was not oxidized, Applicants introduced Cys at the alanine (Ala) 175 position in trastuzumab meditope-enabled monoclonal antibodies (memAb) V2 (I83E). In addition, Applicants synthesized a linear meditope (SEQ. ID NO:4) where X was diphenylalanine and the serine (Ser) at positon 6 was replaced with Cys. Applicants hypothesized that the Ala175Cys mutation in the memAb heavy chain and the Ser6Cys mutation of the meditope would bring the sulfur groups into close proximity, facilitating the formation of a disulfide bond (FIG. 2A).

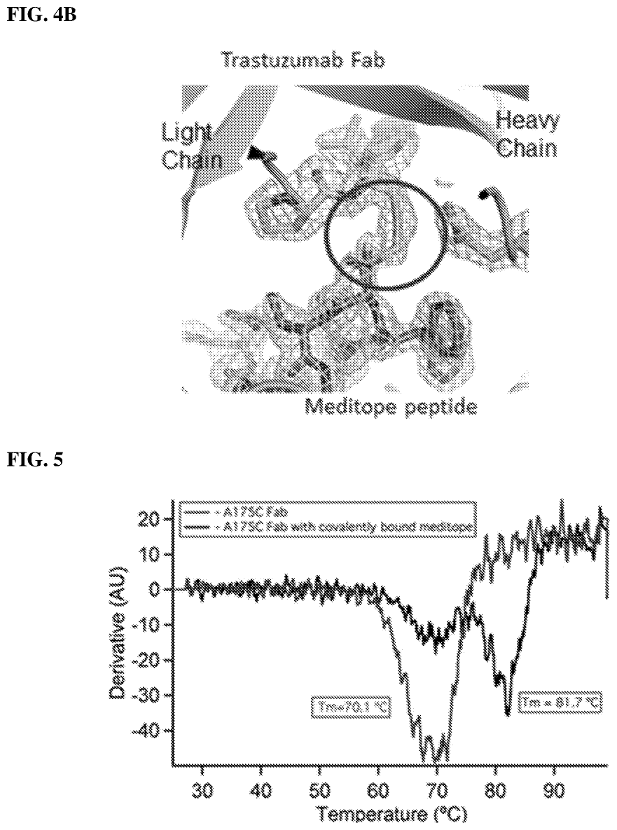

Applicants purified to homogeneity the mutagenized Ala175Cys trastuzumab memAb, and produced Fabs (Cys-meFab) from the IgG. The linear mutagenized Ser6Cys meditope (Cys-meditope) was extensively dialyzed and added to the Cys-meFab. The reaction was allowed to occur with the assumption that oxygen would serve as the reducing agent. Clear evidence of the formation of a disulfide bond was observed in the X-ray crystallographic diffraction data (FIG. 2B and FIG. 4B).

In order to determine the rate of reaction, Applicants used mass spectrometry. Under strongly denaturing conditions, a peak at 49602 AMU was observed, consistent with the formation of a disulfide between the Cys-meFab and linear Cys-meditope. The increase and subsequent plateau in intensity of the 49602 peak over time indicated that the reaction was effectively complete within 3 hours (FIG. 4A).

To better understand the mechanism (e.g., oxygen-mediated reduction), Applicants will run the reaction under anaerobic conditions.

To understand the effect of the Cys mutation on the stability of the meFab, Applicants used differential scanning fluorimetry (DSF) to measure the thermal melting point. The melting temperature of the Cys-modified ‘apo’ Fab (e.g., Cys-meFab without the Cys-meditope) was similar to the memFab V2 (I83E). Combination of the linear Cys-meditope with the Cys-meFab significantly increased the melting point by approximately 12° C. (FIG. 5). Importantly, excess linear Cys-meditope was removed before determining the melting point of the mixture. Applicants observed similar shifts in melting point when testing cyclic, high affinity meditopes and meFab V2 (e.g., no disulfide bonds formed), but the shift was concentration dependent. Thus, this study provides additional evidence that a disulfide bond is formed between Cys-meFabs and Cys-meditopes.

The results of the DSF study further suggest that covalent linkage can serve as a simple but effective means of dramatically improving the stability of monoclonal antibodies (mAbs). Stability correlates with favorable therapeutic properties. This means of improving stability also poses a solution to the ‘cold chain’ problem (e.g., delivery to remote places in the world that do not have refrigeration).

Applicants have demonstrated that natural amino acids can be used to generate a disulfide bond through a templated reaction. Preliminary data suggests that this reaction is oxygen dependent. To facilitate the disulfide interaction, a leaving group, thiopyridine, was added to the thiol of Cys6 in the meditope. Mass spectrometry indicated that the reaction rapidly goes to completion. Specifically, to measure the reaction rate, the thiopyridine-meditope and Cys-Fab were mixed, added to an LC column at different time points to separate Fabs with the disulfide bridged meditope and unreacted, and then analyzed by mass spectrometry. The first time point (e.g., mix and eject -t=0 sec) was fully reacted.

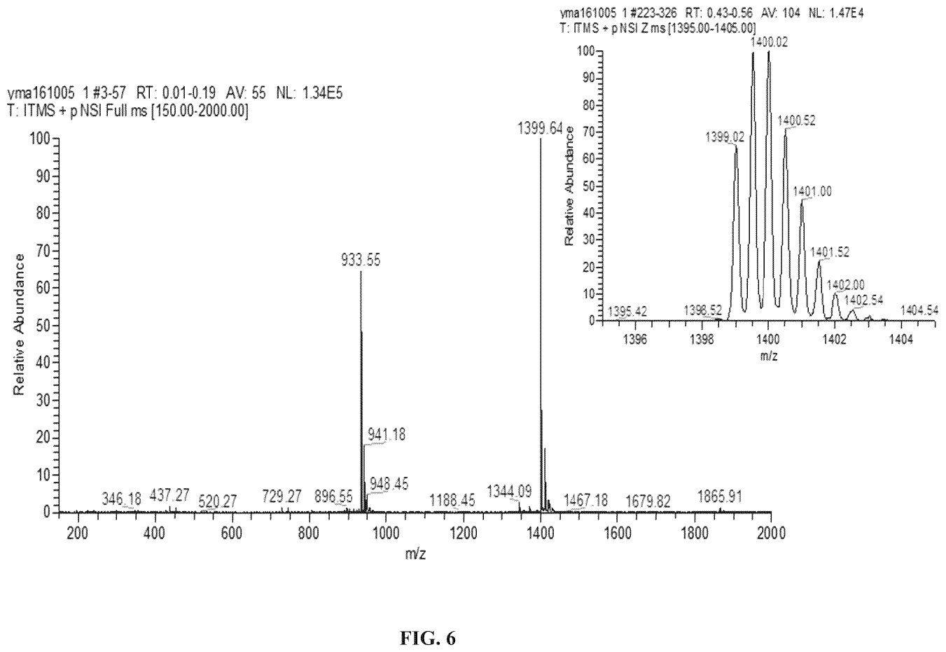

Applicants synthesized an AlexaFluor647 conjugated meditope using the thiopyridine-meditope (SEQ ID NO:1) (FIG. 6).

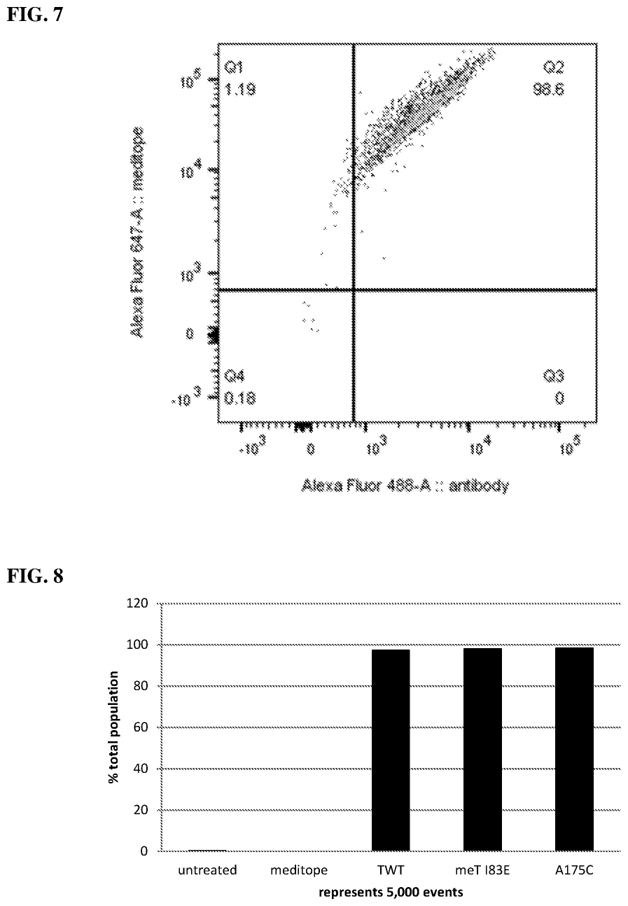

To determine whether the AlexaFuor647 conjugated thiopyridine-meditope/trastuzumab meFab complex could still bind to cells, 10 nM of the A175C meFab conjugated to the AlexaFluor647 thiopyridine-meditope was purified and added to SKBR3 cells for 30 min. The reaction took place on ice. Cells were washed three times and subsequently stained with a secondary antibody (AlexaFluor488). Analysis revealed that covalent linkage of the AlexaFluor647 conjugated thiopyridine-meditope to the meFab did not affect Fab cell binding affinity and, moreover, the labeling was effectively perfect (FIG. 7). Indeed, neither meditope-enablement through conjugation nor templated disulfide binding affected antibody antigen binding (FIG. 8).

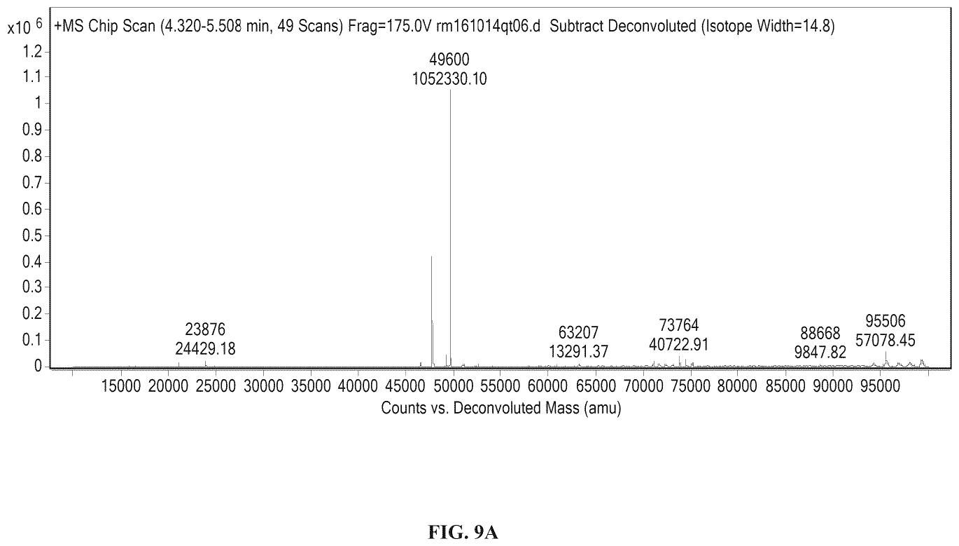

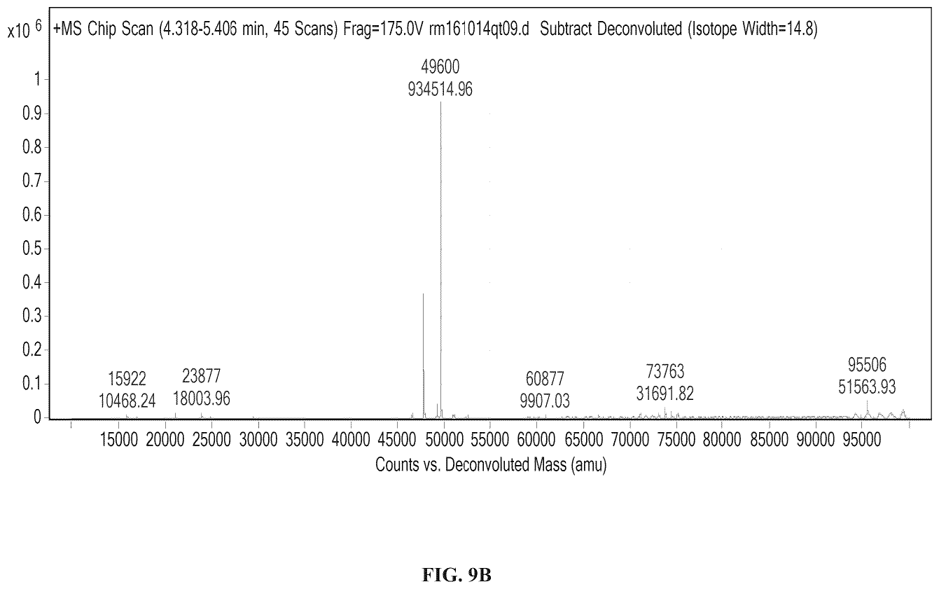

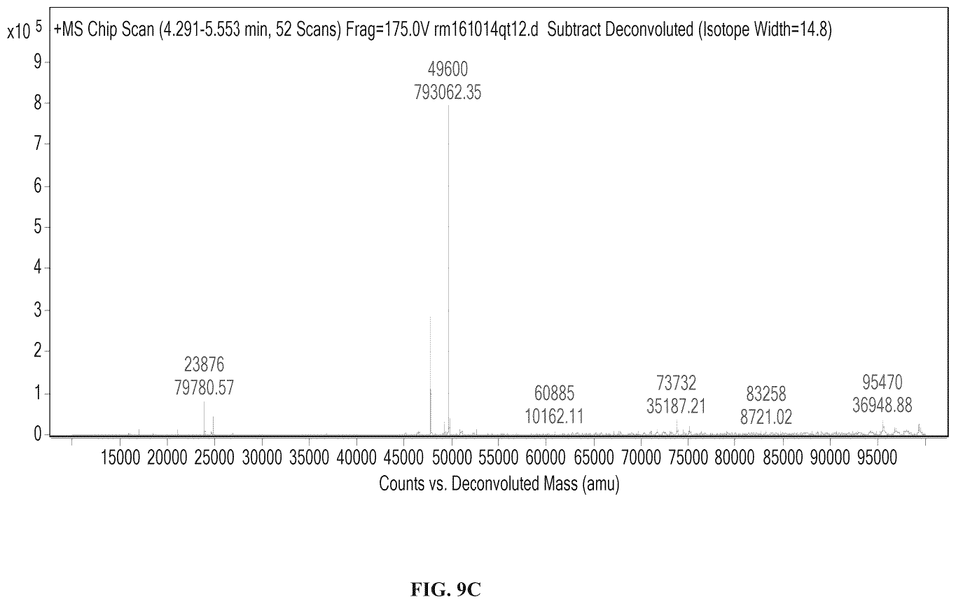

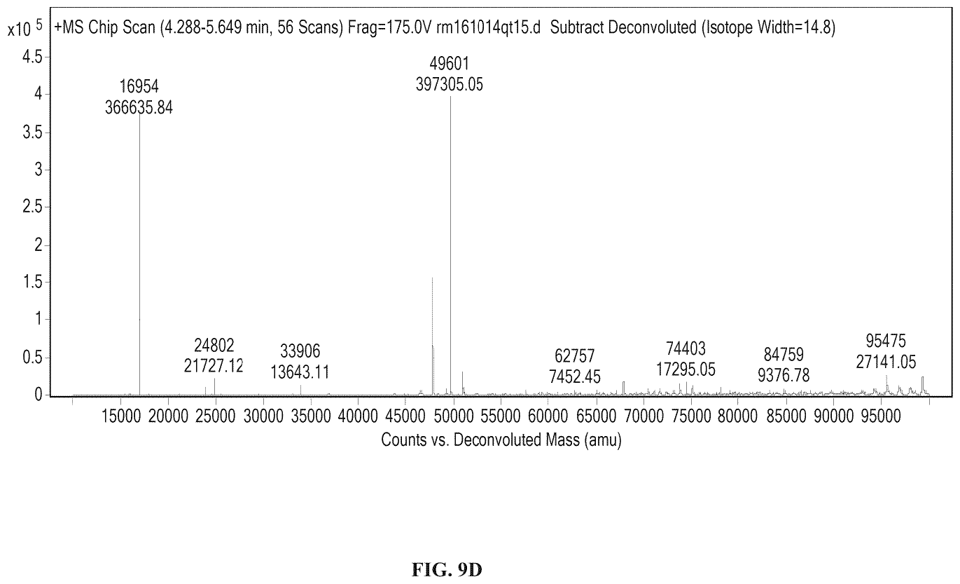

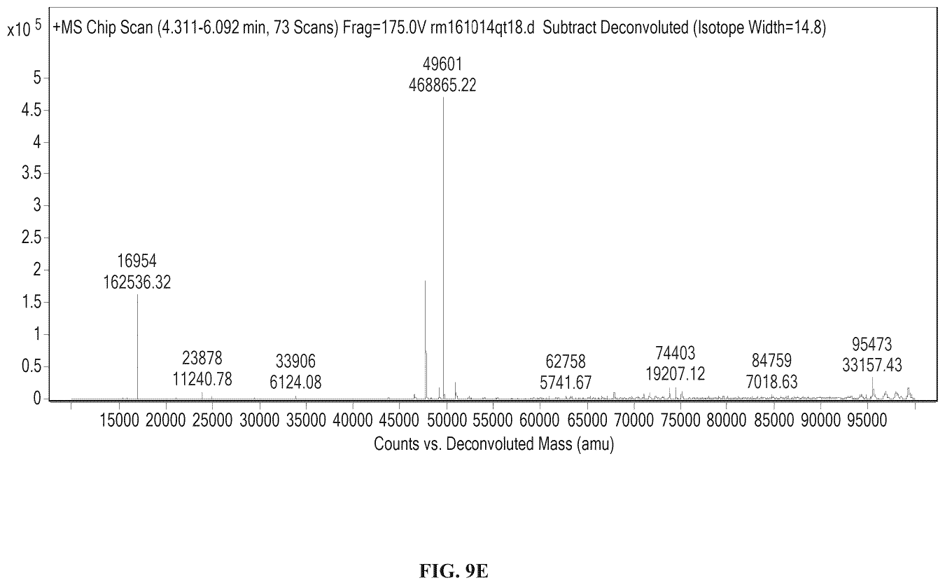

Applicants confirmed that formation of the disulfide bridge between Cys-meditope and Cys-meFab was not pH dependent (FIG. 9A-9E).

The successful formation of the disulfide bond opens up numerous avenues. Importantly, it allows for the creation of biologics with Cys-meditopes. Biologics with Cys-meditopes can then be simply mixed with Cys-memAbs to generate personalized therapeutics. Meditopes will not need to be synthesized with non-natural amino acids, making them useful for developing Fab racks, creating multivalent Fabs with defined geometries (e.g., cyclic trivalent meditope), and addition of biologics that can be readily produced in cell lines.

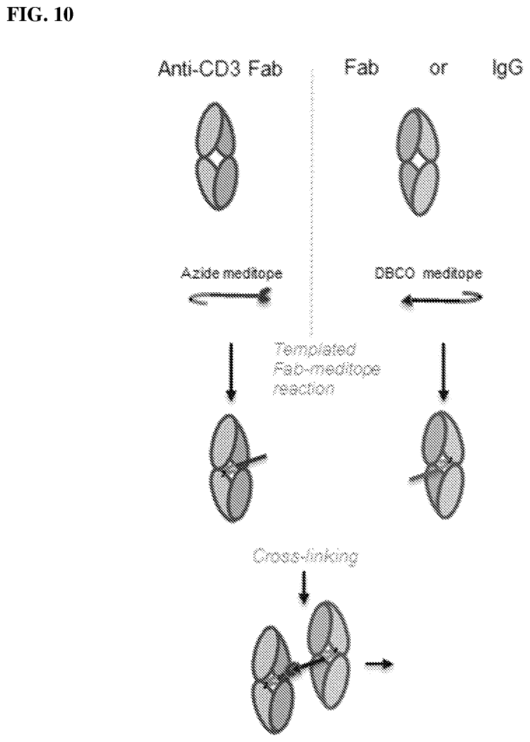

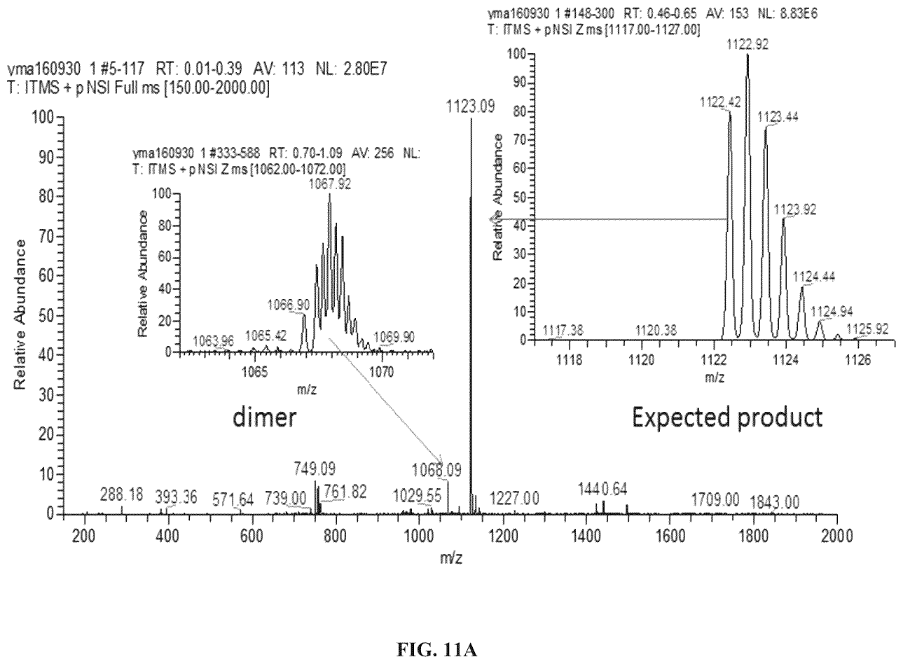

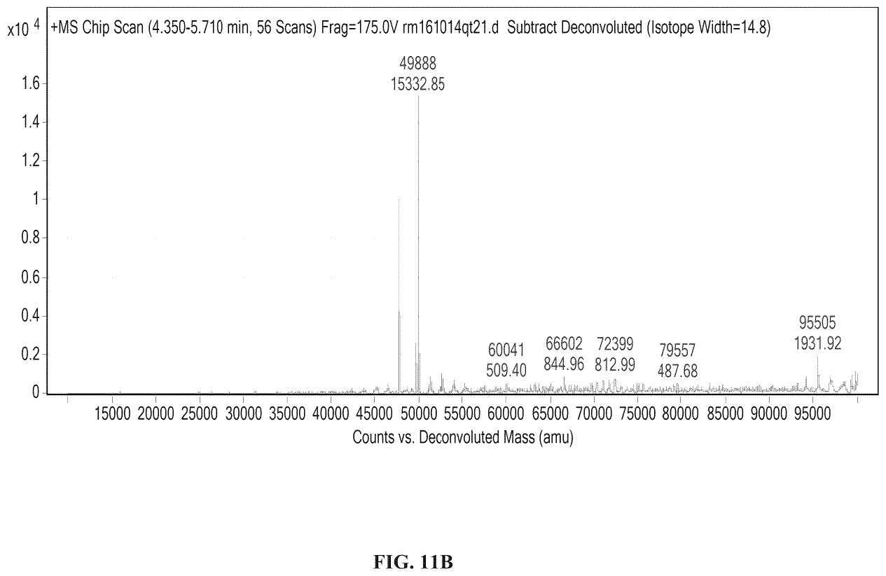

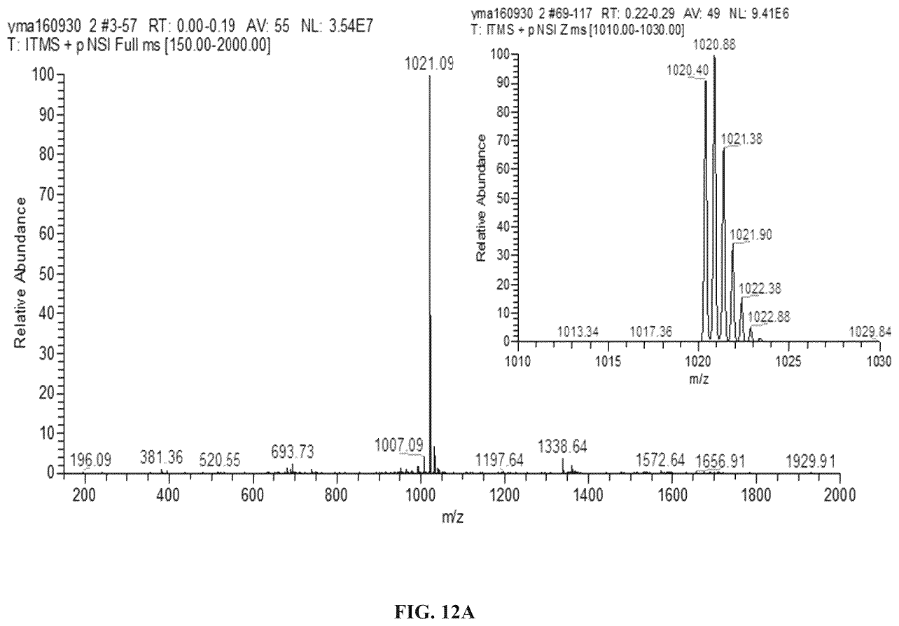

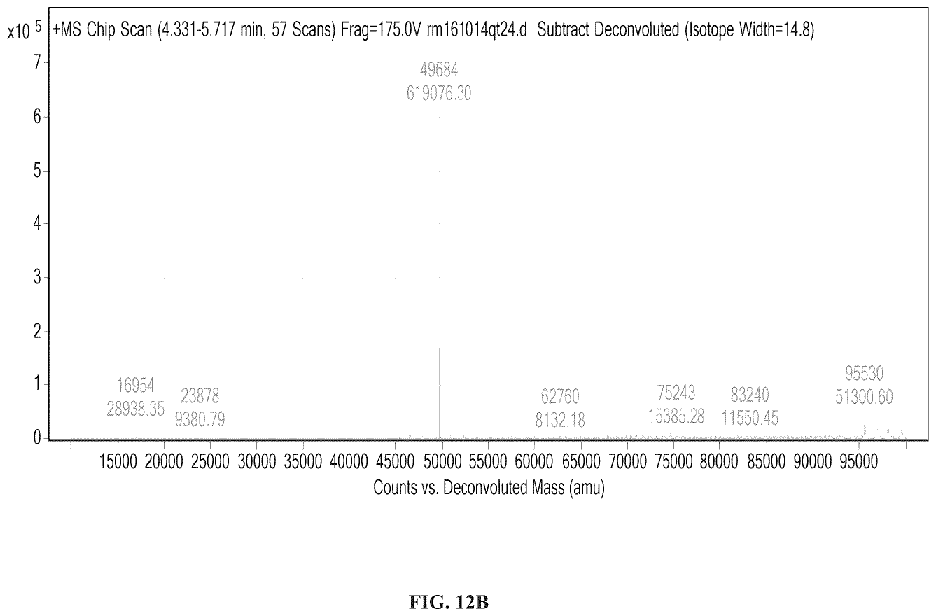

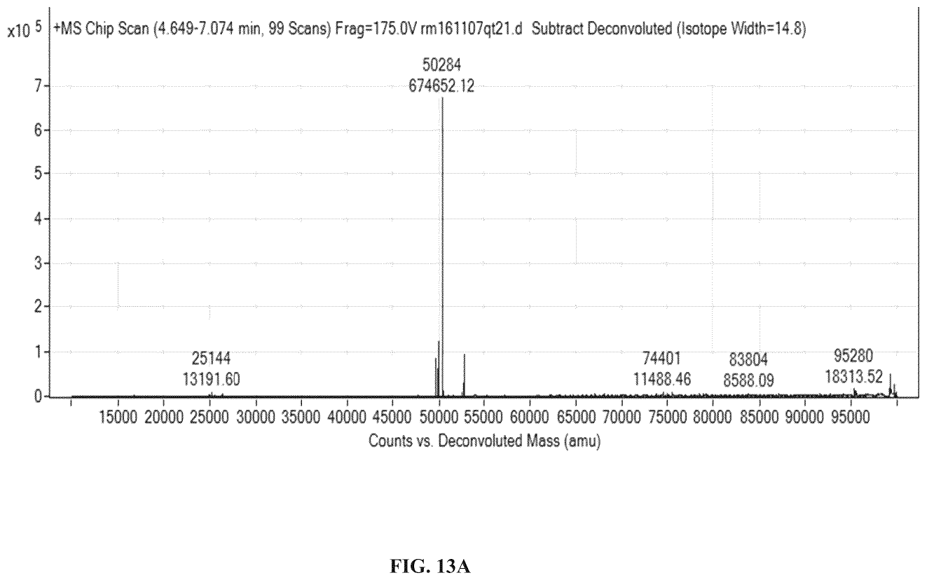

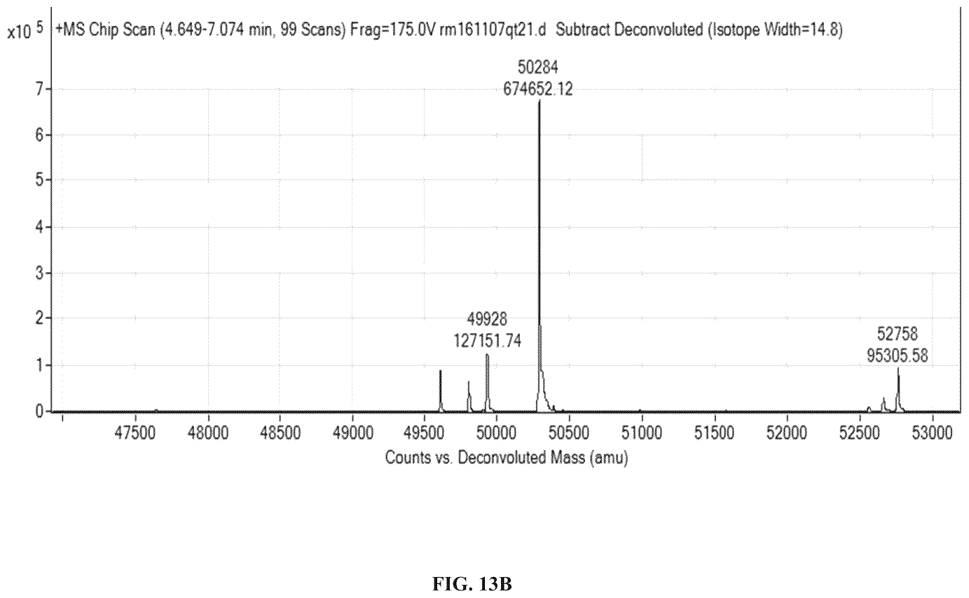

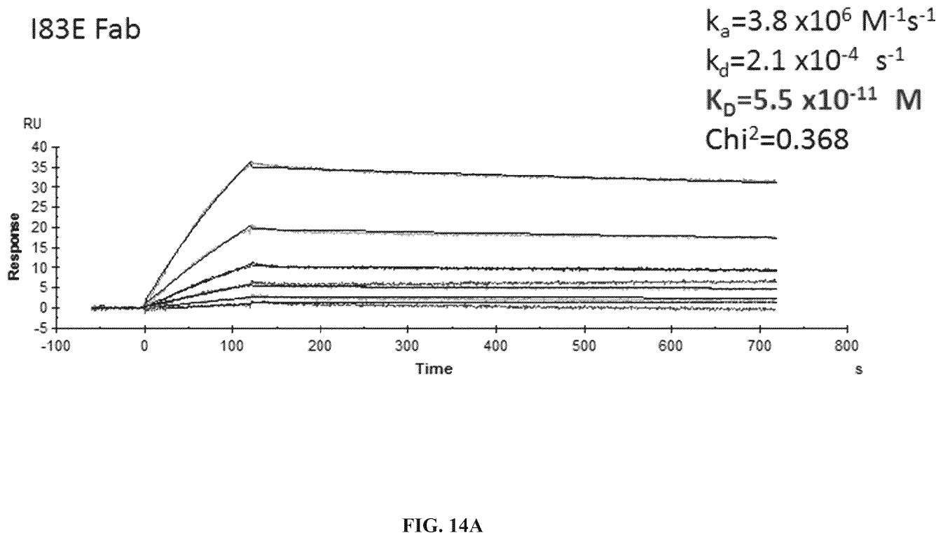

Applicants envision this technology being useful for creating heterodmeric antibodies and/or compositions of Fab fragments, biologics, and drugs (FIG. 10). To facilitate this, Applicants created a DBCO, a strained cyclooctyne that Applicants use for the mechanical bind, meditope (FIG. 11A) and an azide meditope (FIG. 12A). Both conjugated Cys-meditopes were able to bind to the Cys-meFab (FIG. 11B and FIG. 12B). These results indicate that the templated reaction can be used to add unique functionality in an efficient and site specific manner. Applicants hypothesize that in some cases it will be better to add functionality after the templated-disulfide bond is made. Applicants will therefore add different functional group to the pre-formed DBCO/Azido Fabs, including drugs.

Applicants found that they could add cysteines to other sites and use the meditope to direct a thiol to create a disulfide bond. Cysteines can be added to the heavy chain and the light chains of the meFab.

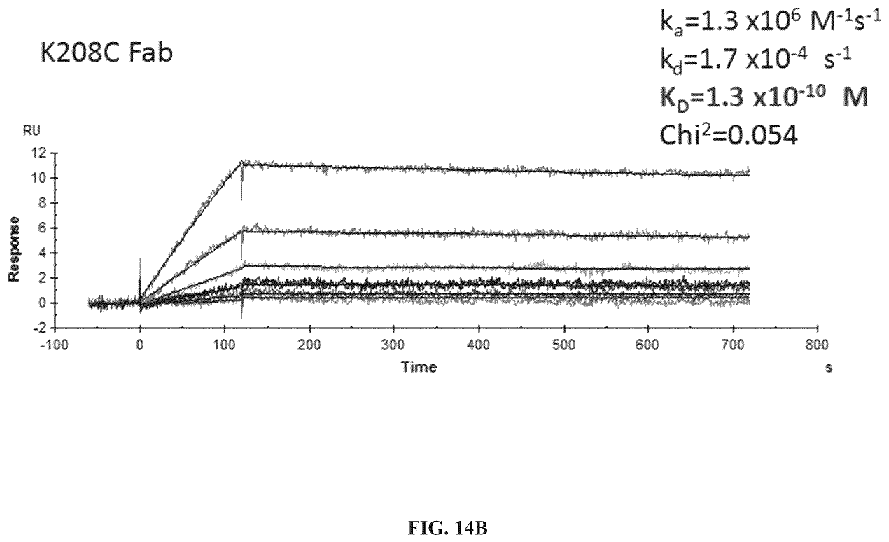

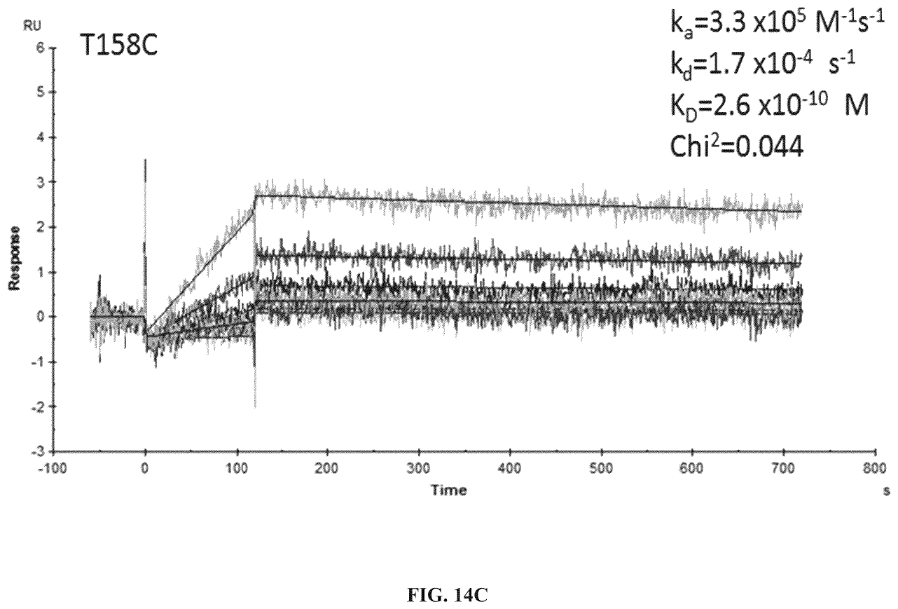

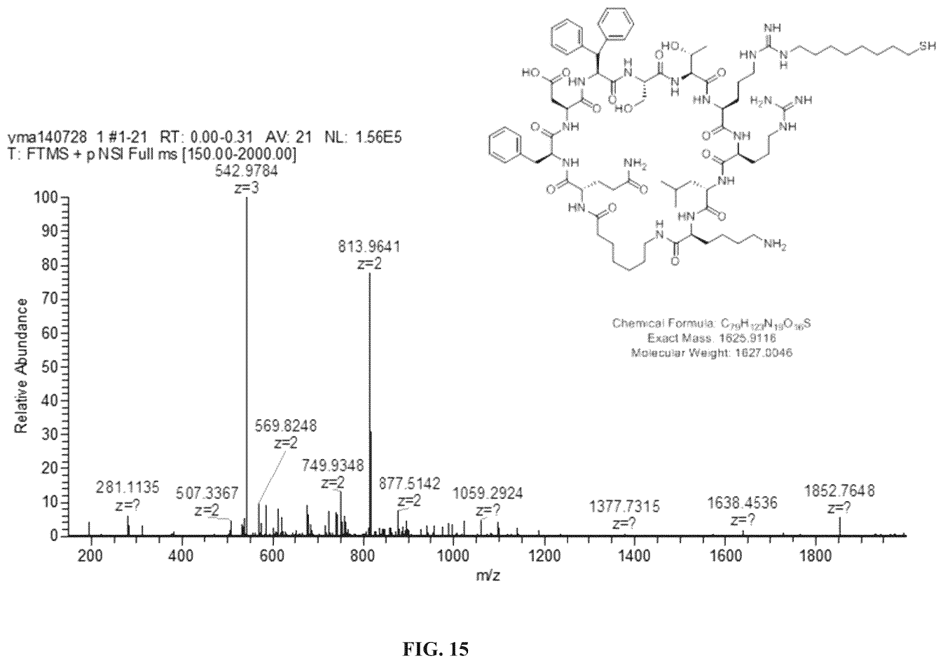

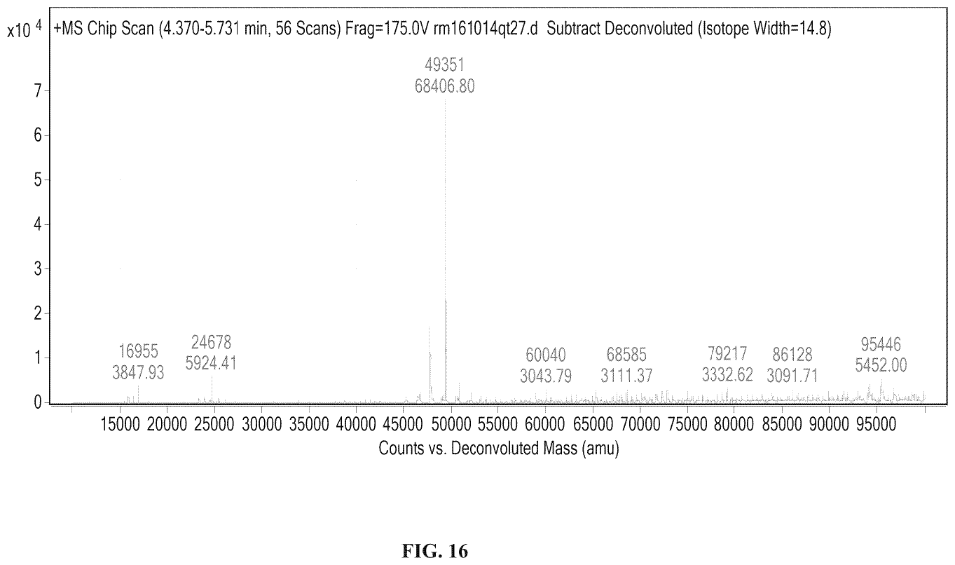

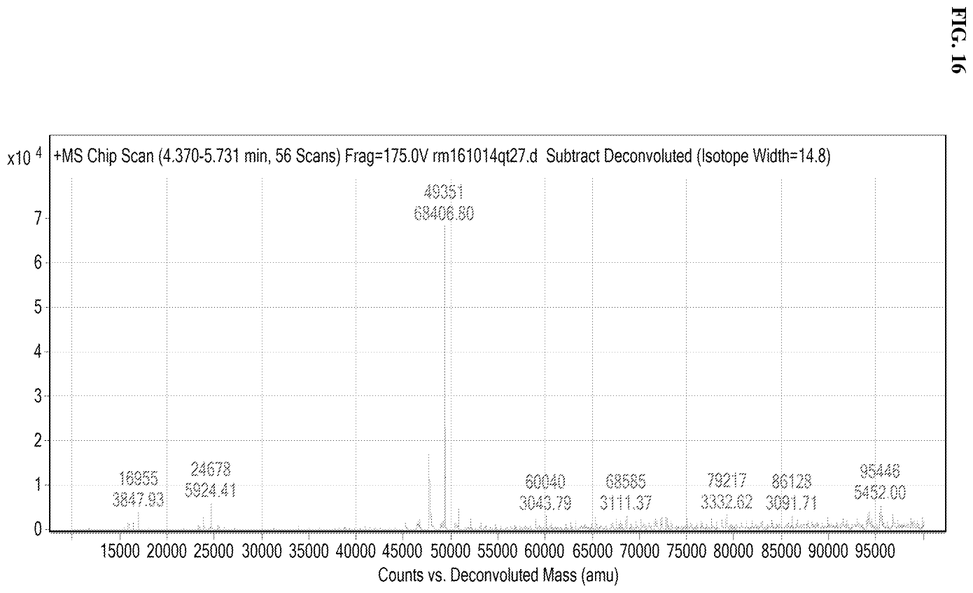

On the back side of the meFab, Applicants created K208C and T158C modifications. Each expressed extremely well, were purified and crystalized. Using SPR to determine how the meFab modifications might affect binding affinity for Her2 revealed that the affinity for the antigen was essentially unaffected (FIG. 14A-14C). In addition, the T158C modified meFabs were able to form a disulfide bond with Cys-meditopes (FIG. 16).

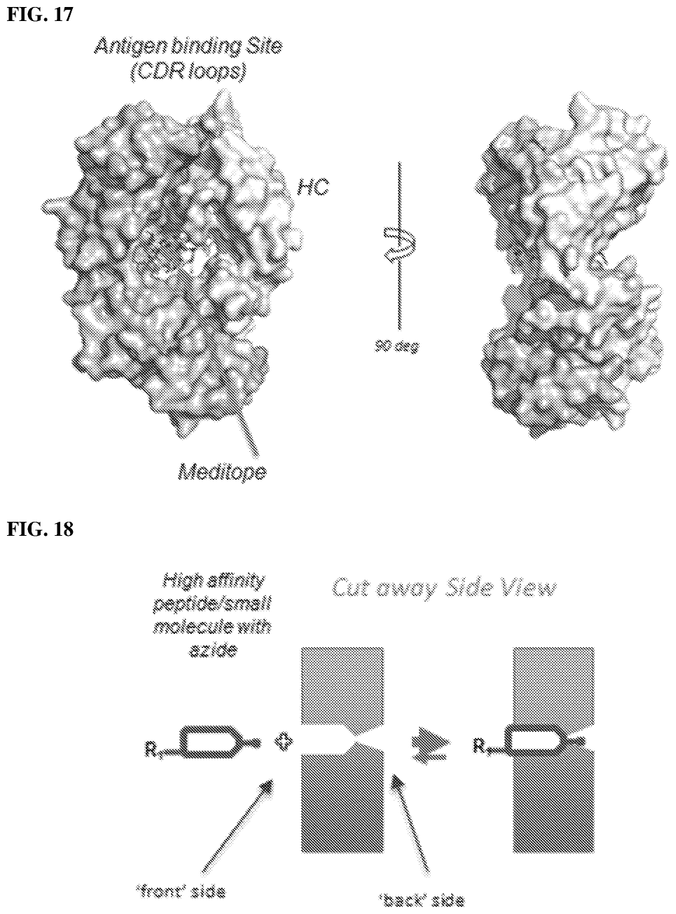

Applicants further demonstrated that they could place an arginine derivative at position 8 of the meditope to thread a thiol through the Fab hole (FIG. 17 and FIG. 18). As before, the close proximity favored the creation of a disulfide bond (as demonstrated for 158C on the heavy chain).

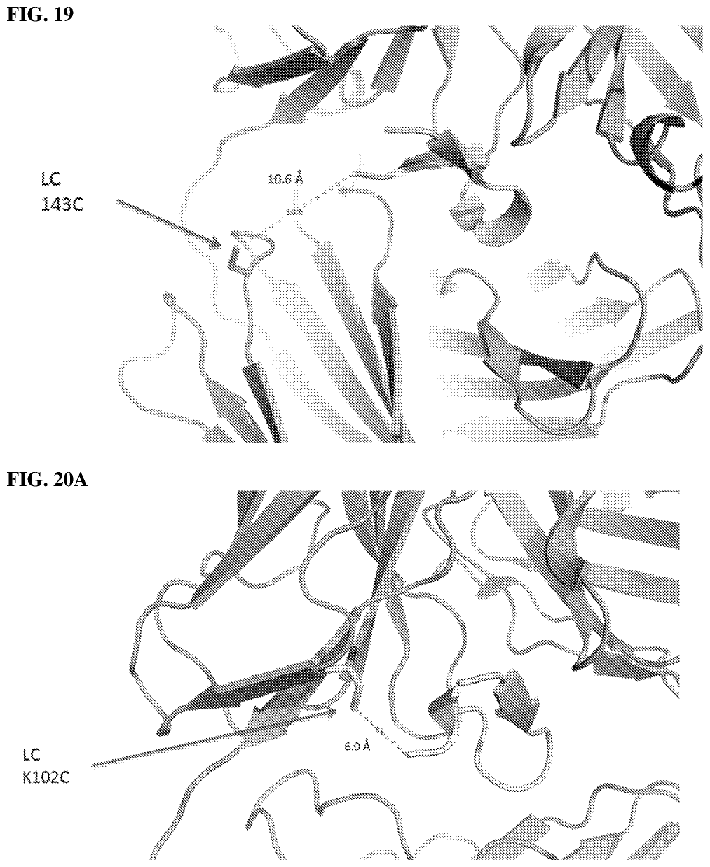

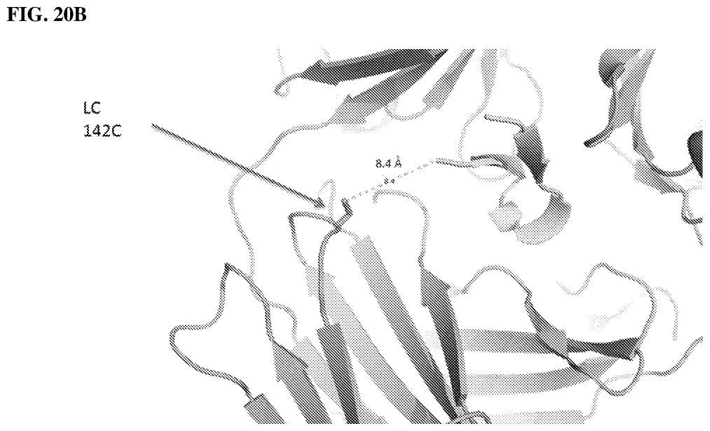

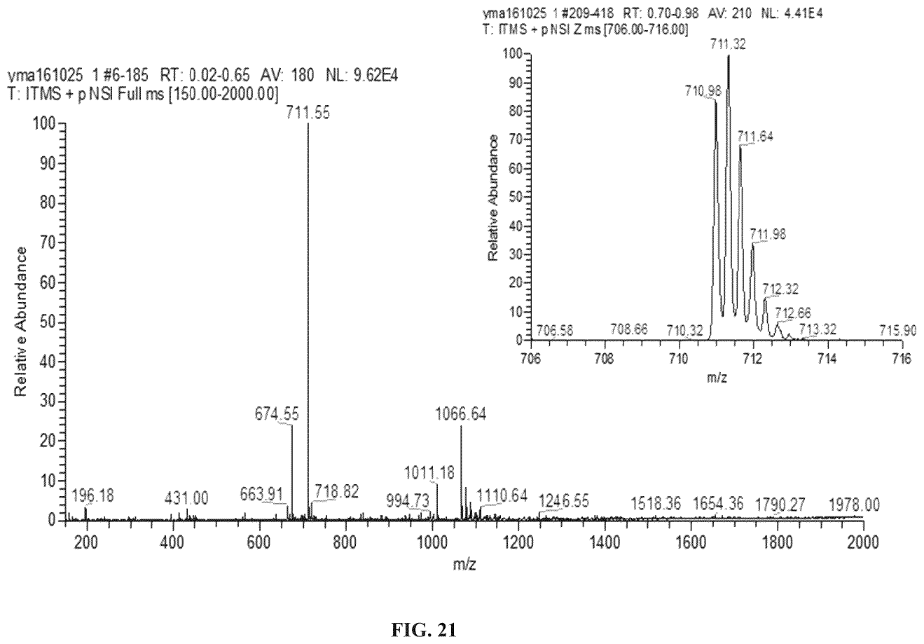

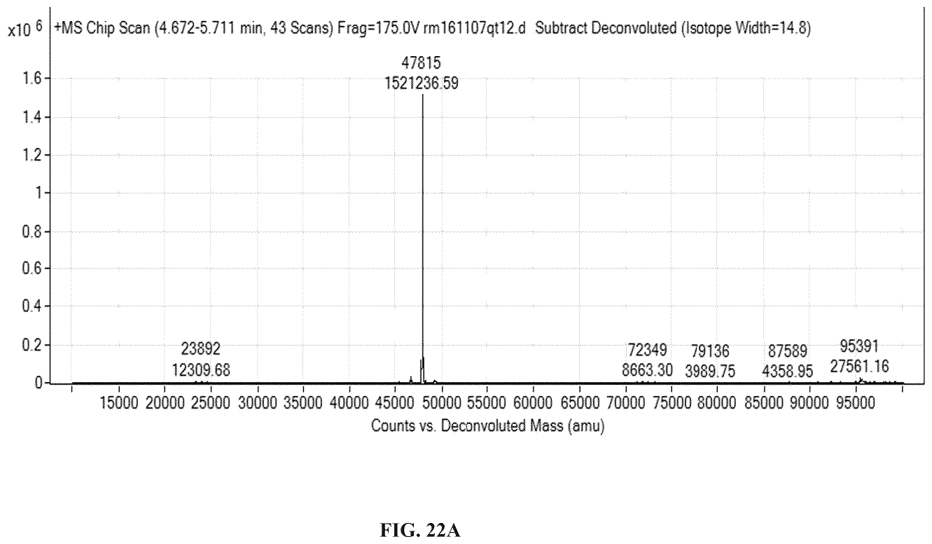

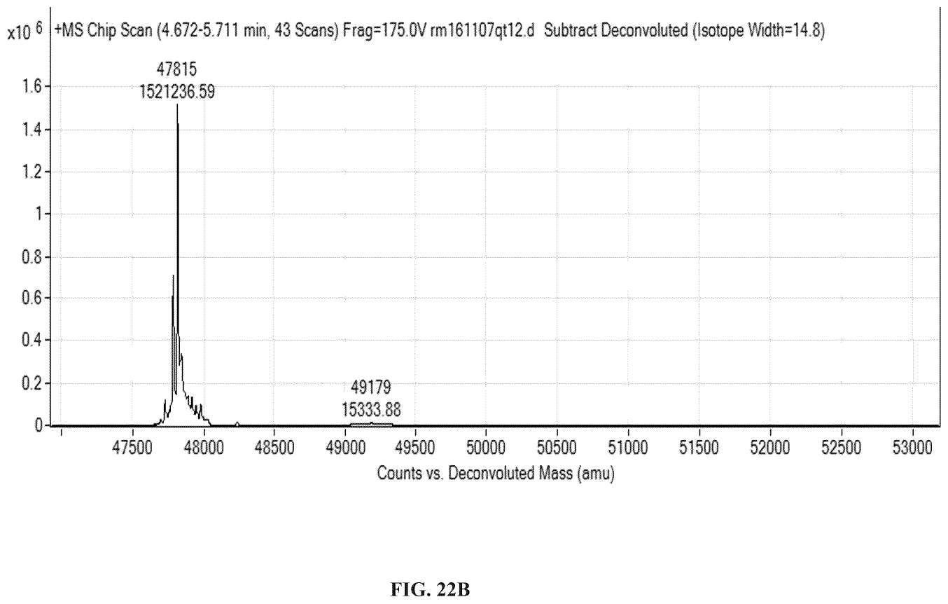

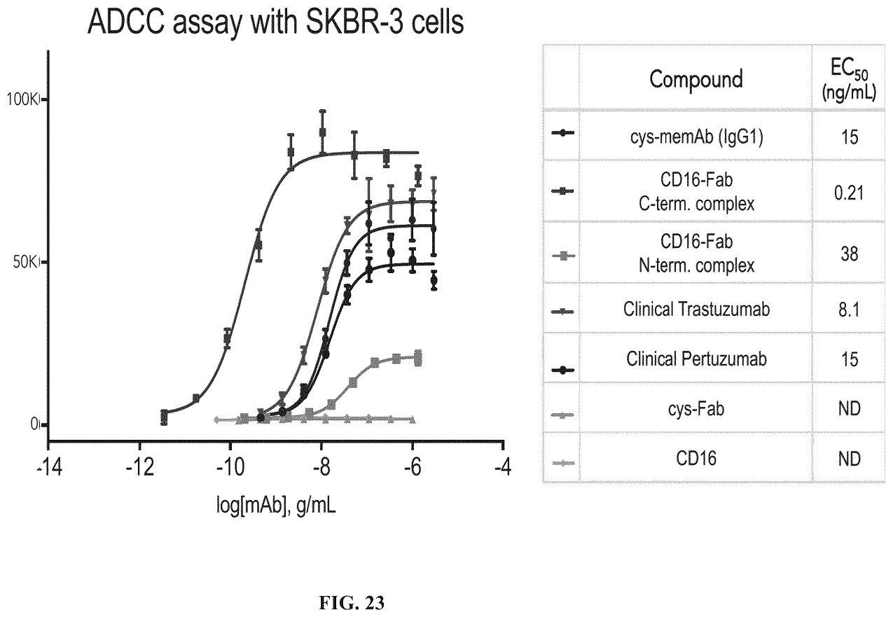

Meditopes can be used to direct disulfides to the meFab light chain (FIG. 20A-20B).

### Example 2: Stable, Site-Specific Modification of Monoclonal Antibodies Using Meditope Peptide-Assisted Disulfide Conjugation

The high specificity and favorable pharmacological properties of monoclonal antibodies (mAbs) have prompted significant interest in re-engineering this class of molecules to enhance their therapeutic and diagnostic potential. Herein, Applicants use the high affinity interaction between a meditope peptide and a meditope-enabled mAb (memAb) to drive the rapid, efficient, and stable site-specific formation of a disulfide bond. Applicants attached fluorescent dyes, cytotoxins, or “click” chemistry handles to memAbs and meFabs using this meditope, peptide-assisted conjugation technology (mPACT) platform. More importantly, Applicants developed genetically-encoded, meditope-tagged biologics to create stable bifunctional Fabs and mAbs. This includes the conjugation of bacterially-expressed fluorescent proteins, nanobodies, and affibodies containing either N-, C- or both terminal meditope tags to memAbs and meFabs. Using the mPACT platform, multiple T-cell and NK-cell—Her2 targeting bispecific molecules were readily created and demonstrated in in vitro assays to potently activate T-cell signaling pathways. Collectively, the mPACT platform offers the opportunity to build and exchange an array of functional moieties including protein biologics among any 175Cys, meditope-enabled mAb and Fab to rapidly create, test and optimize stable, multifunctional biologics.

Monoclonal antibodies (mAbs) and their fragments continue to play a profound role in the current and next generation of therapeutics and diagnostics¹⁻⁵. To leverage their specificity and extend their therapeutic range, a broad array of approaches have been developed over the past 40 plus years. In the broadest terms, there are only two means of functionalizing mAbs—either through chemical conjugation or through genetic engineering. Chemical conjugation extends the therapeutic potential of mAbs through the targeted delivery of small-molecules, toxins, imaging agents, siRNA, and immune modulators⁶⁻⁹. Producing homogenous, functionalized mAbs efficiently, however, remains challenging. Many conjugation methods rely on amine conjugation, resulting in complex heterogeneous mixtures¹⁰. The introduction of unpaired cysteines and non-canonical amino acids has greatly improved these issues, but are mostly limited to small molecules or multiple steps with low yeilds¹¹⁻¹² Alternatively, genetic engineering by fusing additional domains to the underlying mAb provides a means to create new therapeutics with unique mechanisms, such as bispecific T-cell engagers, cytokine fusions, or dual-variable domain mAbs¹³ Optimizing parameters of these genetically fused biologics, such as kinetics, affinity, valency, geometry of receptor engagement, stability, etc., requires the generation of a large panel of constructs. Here, Applicants report an alternative method for the rapid, efficient, and stable conjugation of proteins and small molecules to mAbs.

Applicants recently identified a unique peptide binding site within the Fab arm of cetuximab and demonstrated that the site is absent in humans but can be readily grafted to other mAbs including trastuzumab¹⁴. Since the cyclic, twelve residue peptide binds in a hole that runs through the middle of the Fab arm, Applicants have named it a meditope. Antigen binding is not altered after grafting or in the presence of the meditope¹⁴⁻¹⁵ As such, Applicants understood that they could use the meditope interaction as a hitch for the delivery of cytotoxins, imaging agents, or biologics. The lifetime of the original meditope peptide to cetuximab or the meditope-enabled trastuzumab is seconds at 37° C. Through extensive structure-function studies, Applicants improved the affinity by modifying both the meditope and the Fab, significantly extending the half-life to 40 min at 37° C. Applicants further improved the lifetime by creating a mechanical bond by modifying the arginine at position 8 with an azide, threading it through the Fab hole, and using click chemistry to sterically block its dissociation. While the lifetime of this mechanically interlocked Fab complex is difficult to ascertain, Applicants were capable of imaging tumors in animal xenografts.

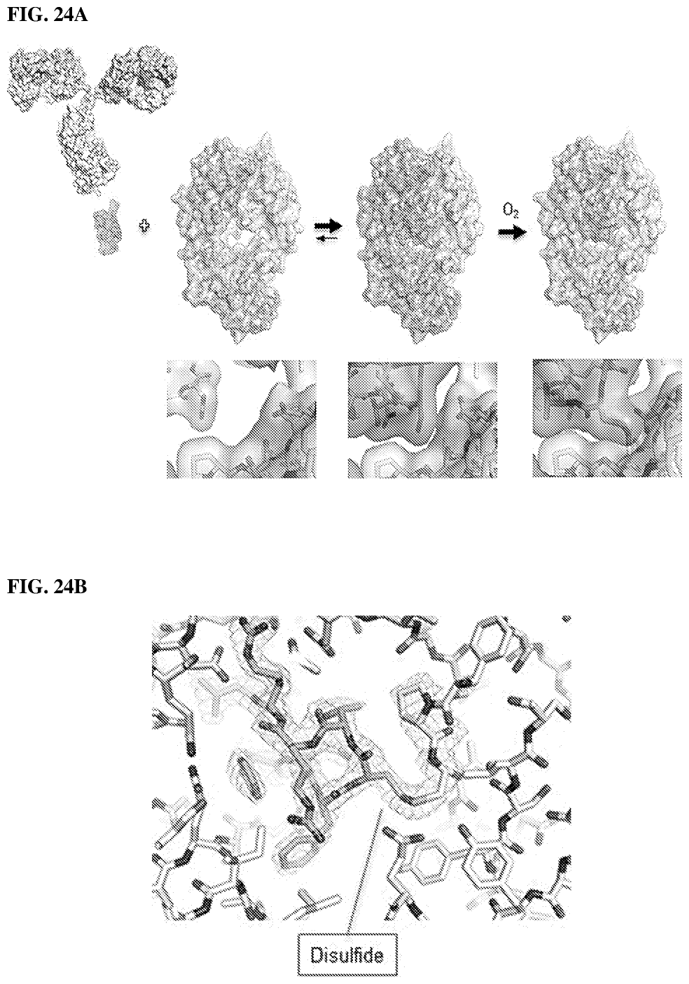

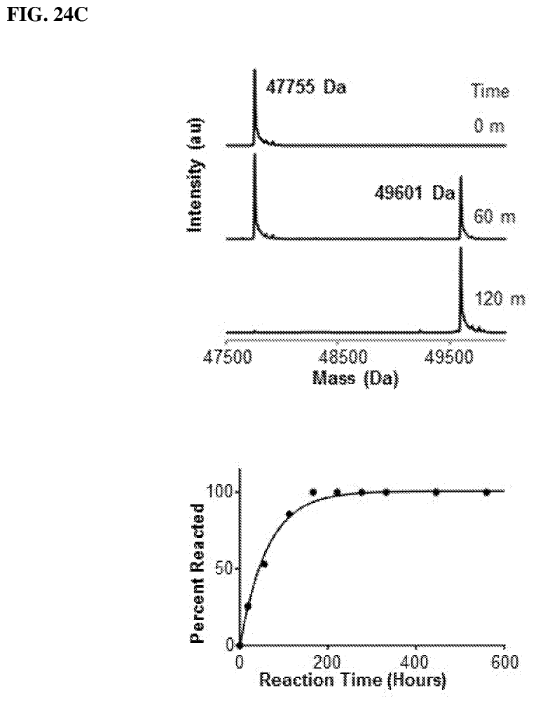

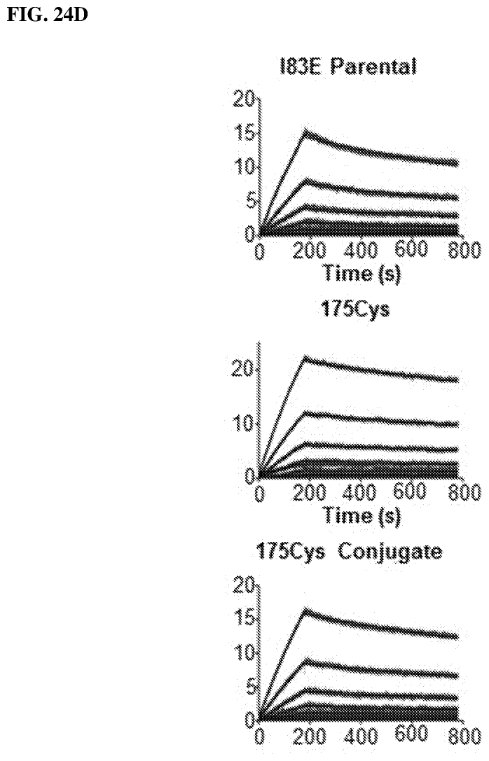

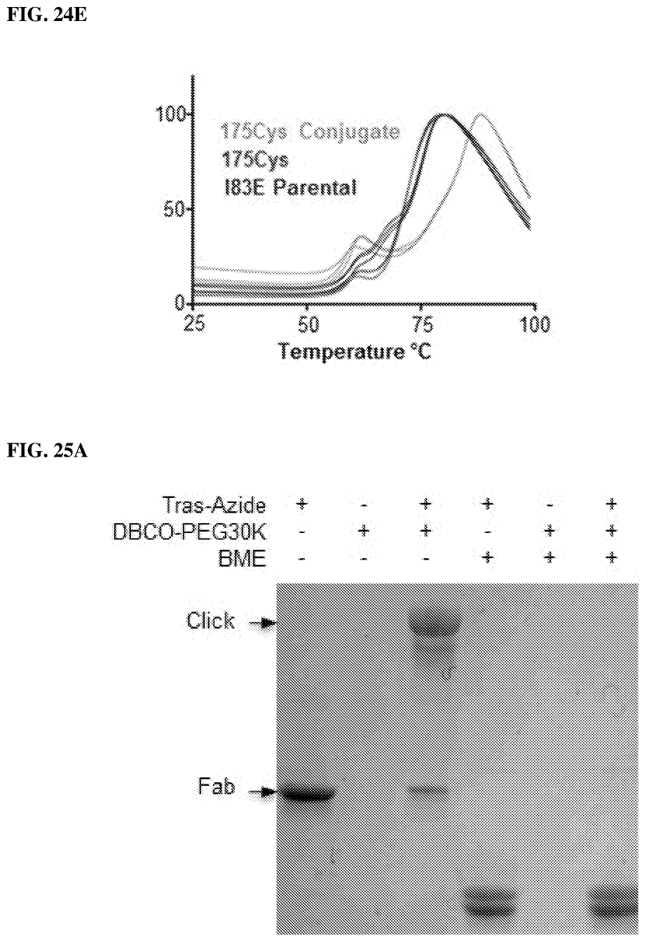

Despite the success of the mechanical bond, its formation required the incorporation of non-natural amino acids. Seeking to eliminate the necessity of subsequent modifications or the incorporation of non-natural amino acids, Applicants asked whether they could use this interaction to drive and subsequently stabilize the formation of a disulfide bond. To this end, Applicants identified Ser6 on the meditope and Ala175 on the heavy chain of the Fab, which are juxtaposed and should be amendable to modification (FIG. 24A). Thus, Applicants mutated Ala175 in the Ile83Glu trastuzumab mAb to cysteine (referred to as 175Cys), produced and purified the modified mAb to homogeneity. To avoid the possibility of mixed-disulfides within the cyclic meditope, Applicants replaced the cysteines at positions 1 and 12 with serines. Ser6 was replaced by cysteine affording a linear meditope, SQFDFCTRRLQSGGSK (SEQ ID NO:22). As Applicants have optimized the crystallization of trastuzumab Fab with meditopes¹⁴, Applicants used crystallography to characterize the formation of the disulfide bond. The crystals diffracted to 2.14 Å and the structure was determined by molecular replacement using the meditope-Fab structure, 4ioi.pdb. Electron density for the meditope peptide including the density for the disulfide bond was clear in the initial maps. The position of the sulfur atoms and the stereo-chemical values of the engineered cysteines in the refined structure were consistent with a disulfide bond (FIG. 24B, FIG. 27). Applicants repeated the above crystallization with a second higher affinity linear cysteine meditope, 5-diphenylalanine (SQFDA(Ph)₂CTRRLQSGGSK; SEQ ID NO:23), to establish a second example of disulfide formation. To further confirm the electron density observed in these structures is consistent with a disulfide bond, Applicants also crystallized, collected diffraction data and solved the structures of the apo-175Cys-Fab and 175Cys-Fab bound to the original meditope (e.g., serine at position 6 (FIG. 27, Table 1)). In both cases, the electron density of the substituted 175Cys was consistent with a reduced sulfur side chain. No additional density was observed between 175Cys of the Fab and Ser6 in the meditope. Consistent with Applicants' previous observations, the overall structure of the Fab from each structure is unperturbed to the parental trastuzumab Fab. The RMSD of each structure to the apo-parental structure is less than 0.68 Å (Table 2).

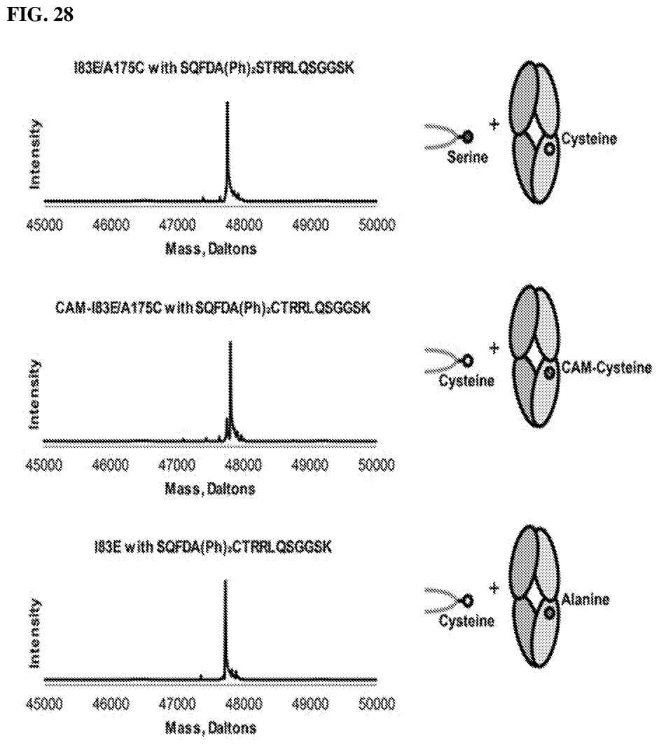

To further validate the diffraction data and better characterize the templated reaction, Applicants monitored disulfide formation between the meditope peptide and 175Cys Fab using LCMS under denaturing conditions. A five-fold excess of the linear cysteine, 5-diphenylalanine meditope (SQFDA(Ph)₂CTRRLQSGGSK; SEQ ID NO:23) was add to the 175Cys Fab and the progression of the reaction was monitored for 16 hours (FIG. 24C, top). At the start of the reaction, the mass of the 175Cys Fab was consistent with the mass of the apo-Fab. The reaction went to completion by 120 minutes, and the mass of the final product matched the mass of the Fab with the meditope (FIG. 24C, bottom). Applicants further confirmed the specificity of the disulfide reaction to 175Cys using meditope and Fab combinations lacking their respective thiols, and thus, incapable of forming a disulfide (FIG. 28). Parental Ile83Glu Fab (i.e., no cysteine) did not react with SQFDA(Ph)₂CTRRLQSGGSK (SEQ ID NO:23), nor did 175Cys Fab with the serine meditope variant SQFDA(Ph)₂STRRLQSGGSK (SEQ ID NO:24). In addition, blocking the 175Cys Fab thiol with iodoacetamide completely prevented the reaction with SQFDA(Ph)₂CTRRLQSGGSK (SEQ ID NO:23; FIG. 28).

To confirm the formation of the disulfide does not perturb the antigen binding or the overall stability of the Fab, Applicants conducted surface plasmon resonance (SPR) and thermal shift measurements. With soluble extracellular domain of HER2 coupled to the SPR sensor chip, the kinetics and affinity of the Fabs: Ile83Glu, 175Cys, and 175Cys conjugated to SQFDA(Ph)₂CTRRLQSGGSK (SEQ ID NO:23), were determined (FIG. 24D). No discernable differences of the calculated on-rate (kₐ) or off-rate (k_{d}) were observed among different Fabs, consistent with Applicants' previous observations (FIG. 24D, Table 3).

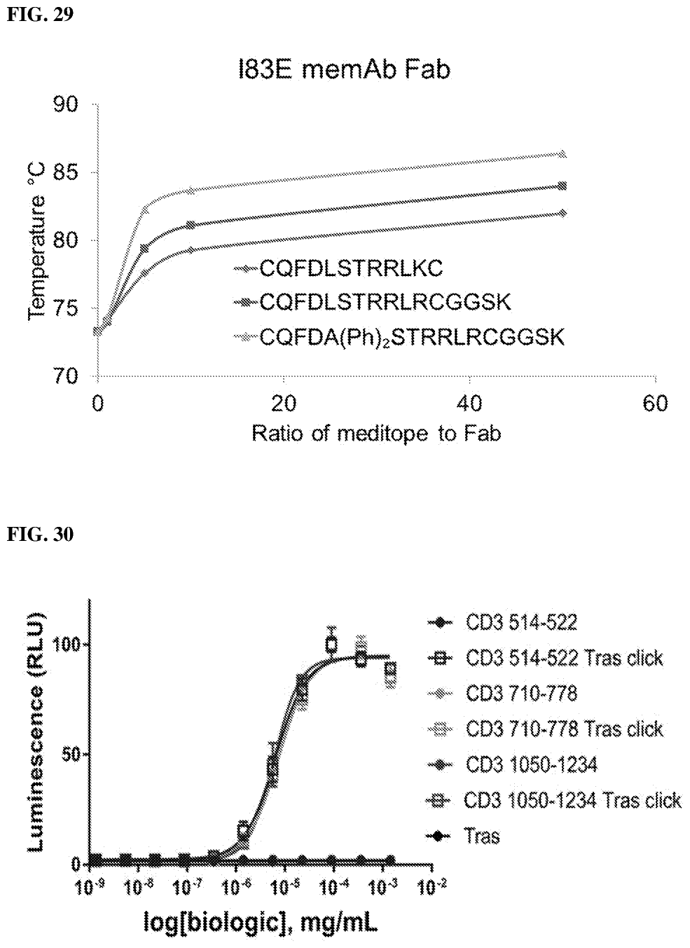

Furthermore, Applicants conducted differential scanning fluorimetry to characterize the effect of the cysteine mutation on mAb stability. The melting temperature of the 175Cys variant (Tₘ=70.9° C.) is similar to the original meditope-enabled mAb (Tm=70.8° C.) (FIG. 24E). The stability of the 175Cys Fab-SQFDA(Ph)₂CTRRLQSGGSK (SEQ ID NO:23) complex was substantially higher, Tₘ=81.2° C. This 10° C. increase in thermal stability likely reflects favorable interactions between the meditope and the Fab framework as Applicants have observed a similar increase in the melting temperature using the non-cysteine meditope with the Ile83Glu Fab. However, a 10-50 fold molar excess of the meditope was necessary to produce a similar shift (FIG. 29).

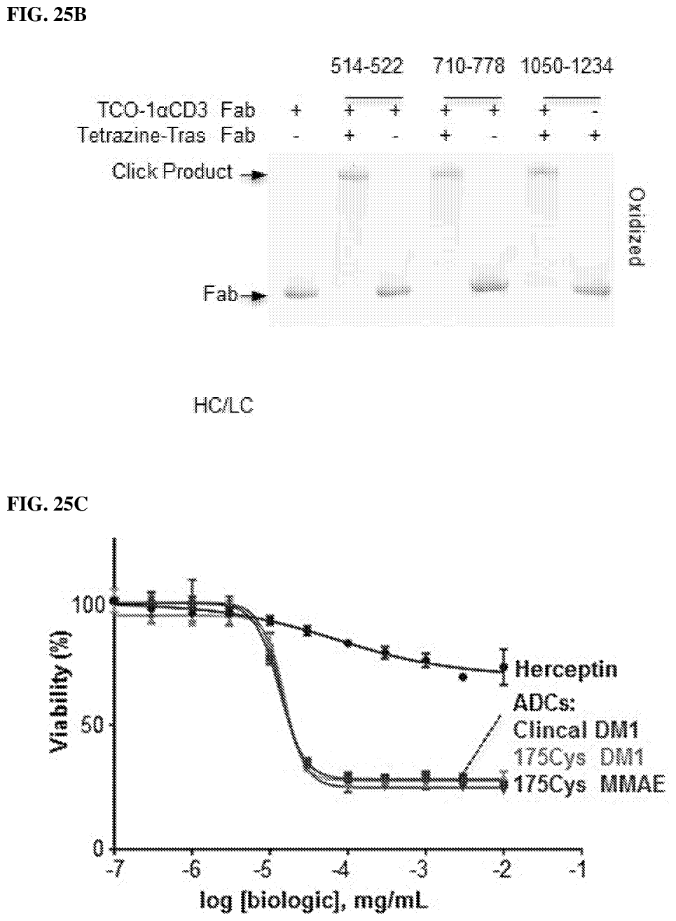

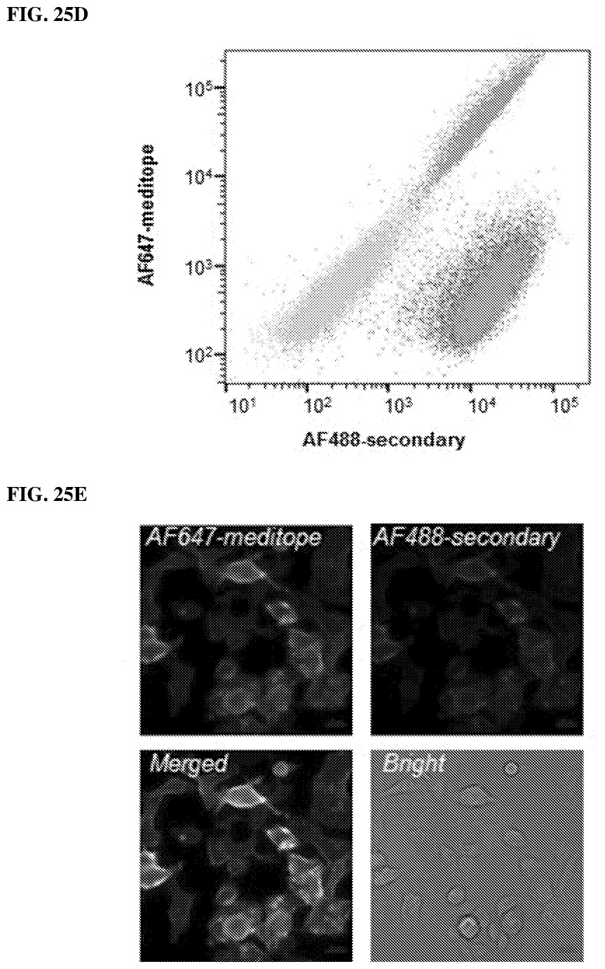

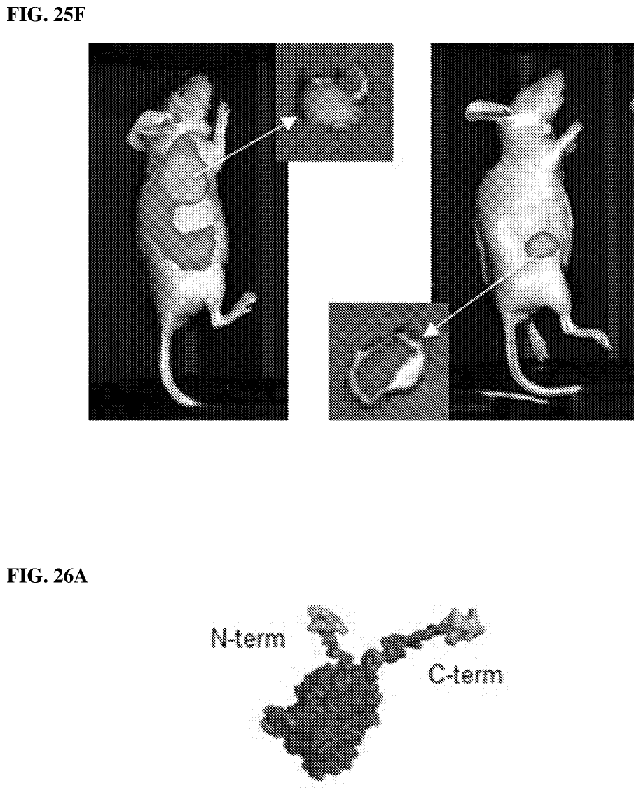

Since the templated disulfide reaction is site-specific and rapid, Applicants asked whether they could use it to attach small molecules to 175Cys memAbs/Fabs. Applicants conjugated azide, trans-Cyclooctene (TCO), or tetrazine to the terminal lysine of the acetylated SQFDA(Ph)₂CTRRLQSGGSK (SEQ ID NO:28) for click chemistry. First, Applicants reacted the azido-cysteine meditope with 175Cys Fab. Next, Applicants used click chemistry to add a 30 kDa pegylated-DBCO to the azido-175Cys Fab. As shown by SDS-PAGE, a 30 kDa increase in mass was observed under non-reducing conditions after reacting for 16 hours (FIG. 25A). Upon reduction, the disulfide bonds between the meditope and Fab and between the light and heavy chains are reduced. SDS-PAGE shows that the light chain and heavy chains of the ‘clicked’ DBCO-175Cys Fab run at the same mass as the unreacted azido-A175C Fab.

As the azido-DBCO click chemistry was successful, Applicants tested if Applicants could use the alternative TCO-tetrazine click to create bispecific molecules. Applicants generated three meditope-enabled 175Cys αCD3 Fabs. Applicants independently mixed the tetrazine-cysteine meditope with the 175Cys trastuzumab Fab and the TCO-cysteine meditope with the 175Cys αCD3 Fabs. Applicants separately combined the tetrazine containing trastuzumab Fab with each of the TCO containing αCD3 Fabs. The “clicked” products were purified and confirmed by SDS-PAGE. A band with a mass of −100 kDa was observed (FIG. 25B). The individual meditope-conjugated Fabs were each ˜50 kD. As before, under strong reducing conditions, the ˜100 kD complex dissociated into bands consistent with the light and heavy chains of the Fab. The clicked BiTEs were able to activate luciferase expressing Jurkat cells in the presence of SKBR3 cells (FIG. 30). The EC₅₀ were 66, 77, and 62 pM for the 514-522, 710-778, and 1050-1234 click conjugates, respectively.

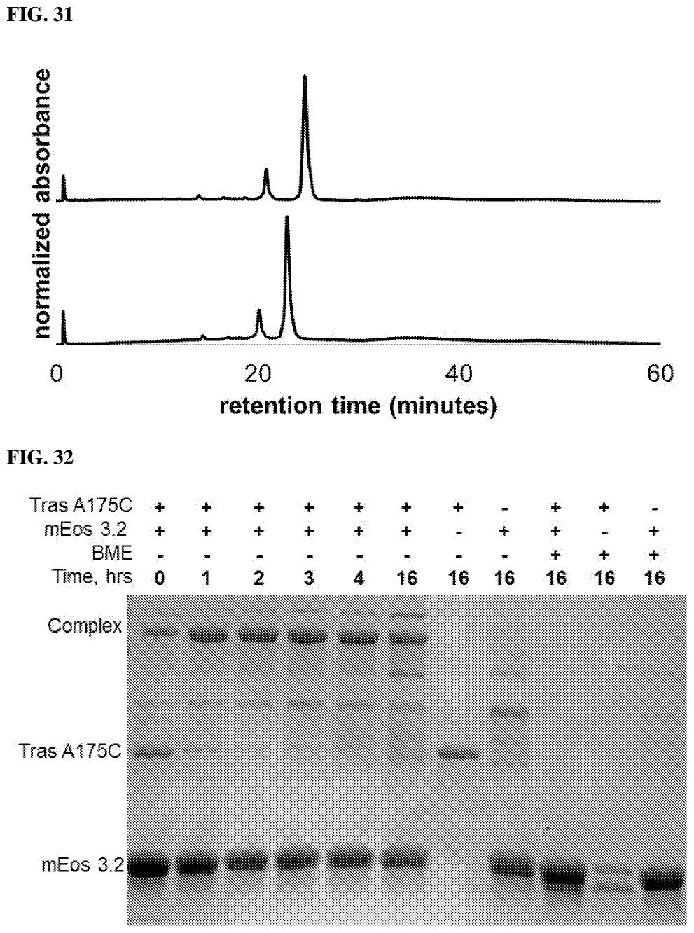

Next, Applicants demonstrated that the templated bond could be used to create an antibody-drug conjugate. Mertansine (DM1) and monomethyl auristatin E (MMAE) were added to the acetylated SQFDA(Ph)₂CTRRLQSGGSK (SEQ ID NO:28) meditope through the lysine amine. A five-fold excess of DM1-cysteine meditope and MMAE-cysteine meditope products were reacted individually with 175Cys trastuzumab memAb yielding two different drug conjugates, both with Drug-Antibody Ratio (DAR) of 1.84 (FIG. 31). Increasing the amount of drug-cysteine meditope did not improve yield beyond 1.9 (Table 4). The activity of both drug conjugates was tested using SKBR3 cells, and compared to trastuzumab emtansine, the clinical antibody-drug conjugate (FIG. 25C). Despite trastuzumab emtansine having an average DAR of 3.4, the EC₅₀ of the meditope-based DM1 and MMAE conjugates were similar (175Cys-DM1 EC₅₀=107 pM; 175Cys-MMAE EC₅₀=100 pM; clinical DM1 EC₅₀=93 pM).

To further explore the utility of the templated disulfide reaction, Applicants conjugated Alexa Fluor 647 (AF647) to the lysine of the acetylated SQFDA(Ph)₂CTRRLQSGGSK (SEQ ID NO:28) meditope. This AF647-meditope was reacted with 175Cys trastuzumab memAb, yielding a dye-antibody ratio of 1.8. Analytical cytometry and fluorescent microscopy indicated specific and robust antigen binding (FIGS. 25D and 25E). Next, Applicants imaged tumors xenografts in four NSG mice. The mice were imaged 24 hours post-injection in an intact animal. Subsequently, the mice were humanely euthanized and the organs were harvested (FIG. 25F). The AF647-175Cys conjugated mAb bound to tumors in all but one mouse. Fluorescent signal was also observed in the GI tract, a common observation in imaging studies¹⁶. Additional studies are underway to optimize the signal-to-noise and to add different fluorophores to understand their role on biodistribution. These data, nonetheless, indicate that the templated disulfide bond provides a versatile approach to functionalize mAbs with small molecules and bio-orthogonal chemical moieties.

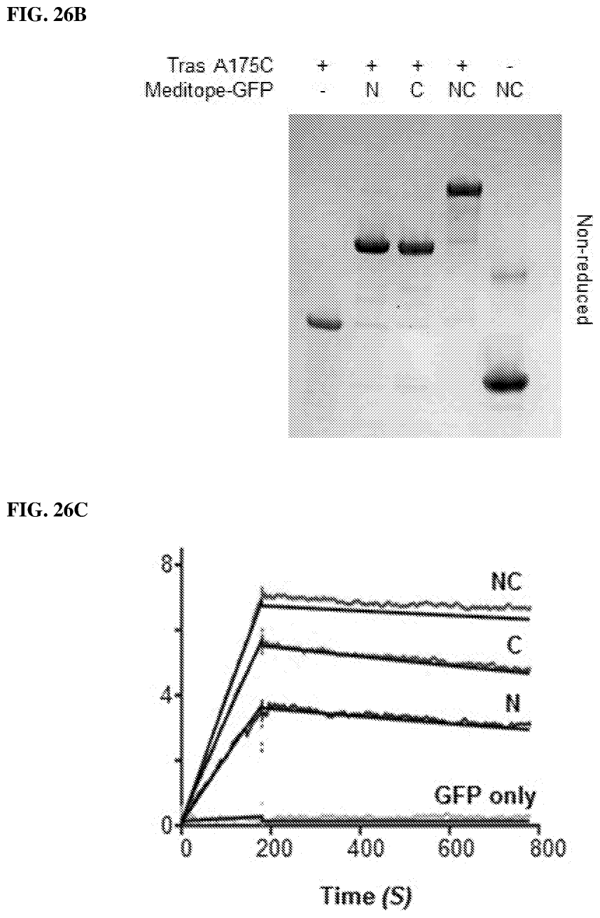

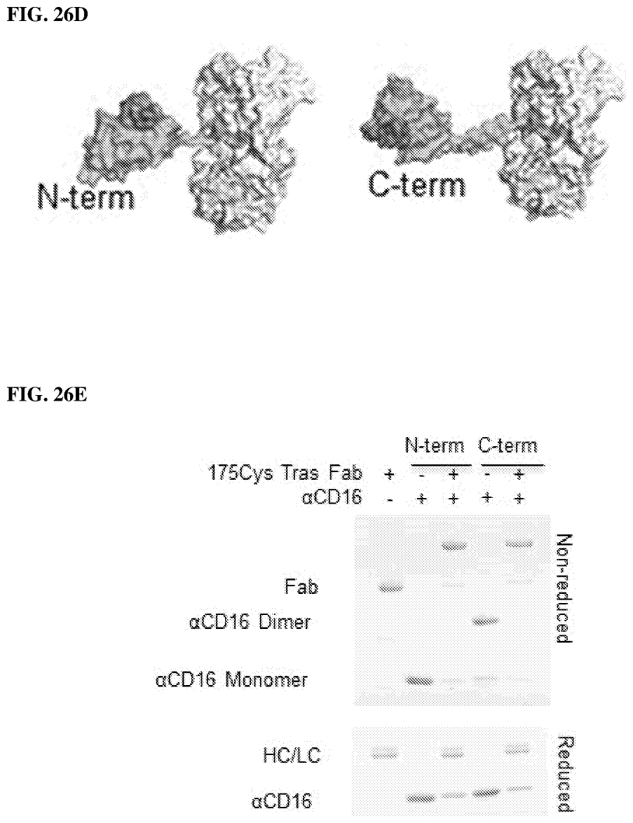

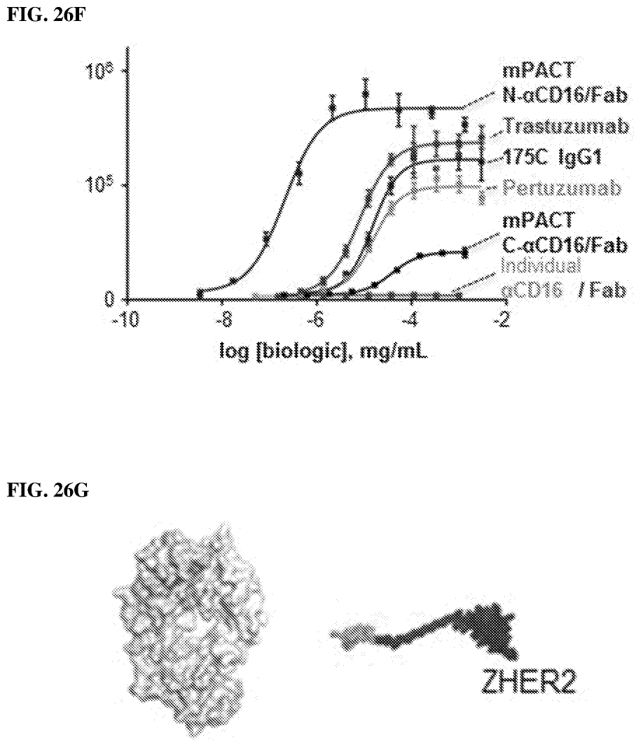

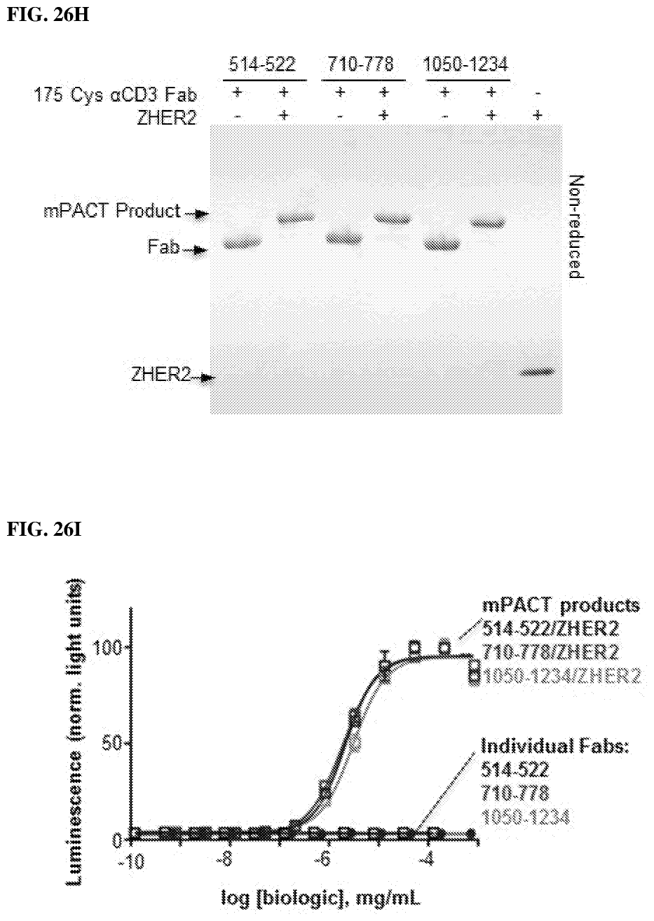

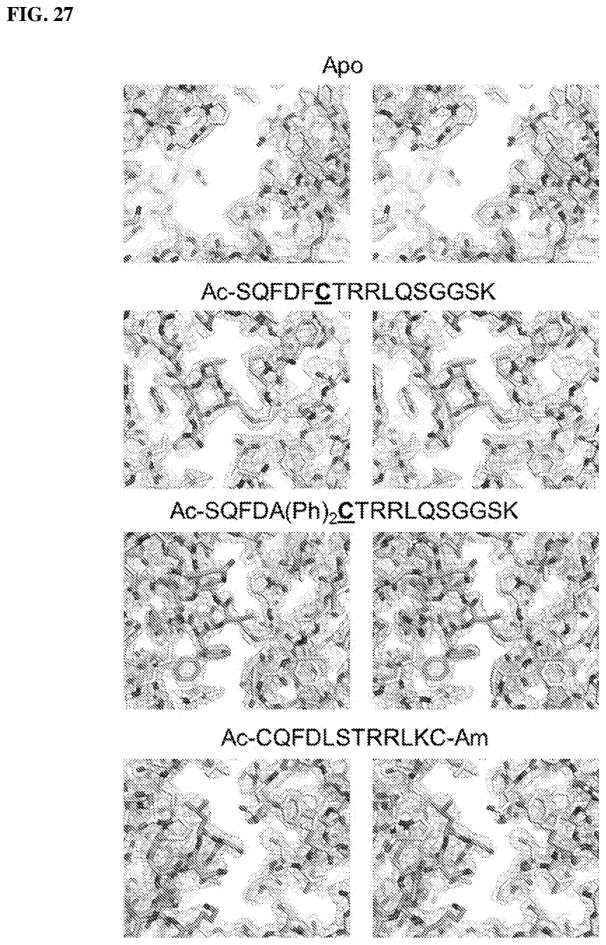

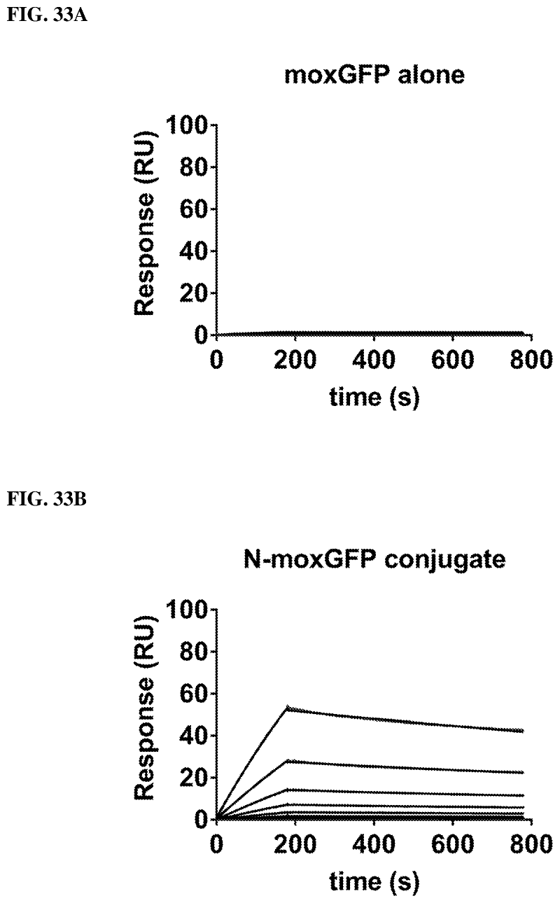

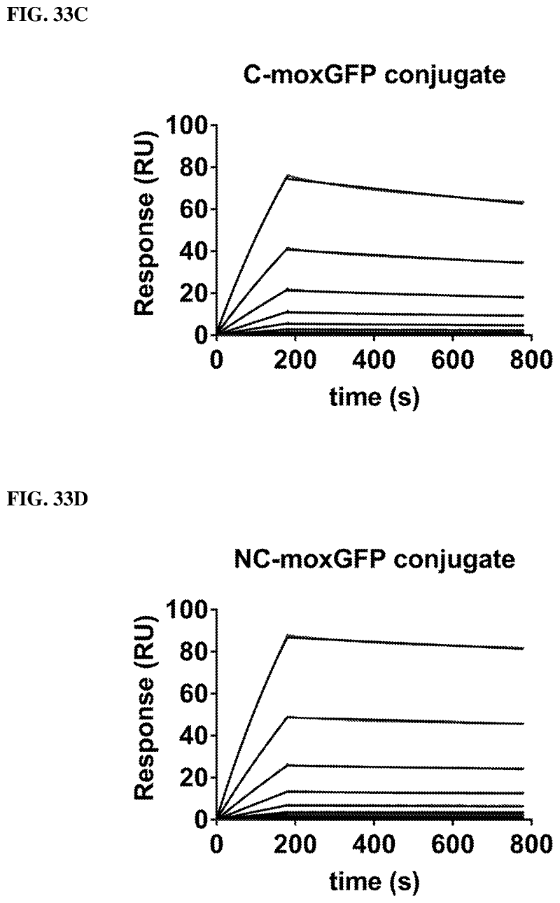

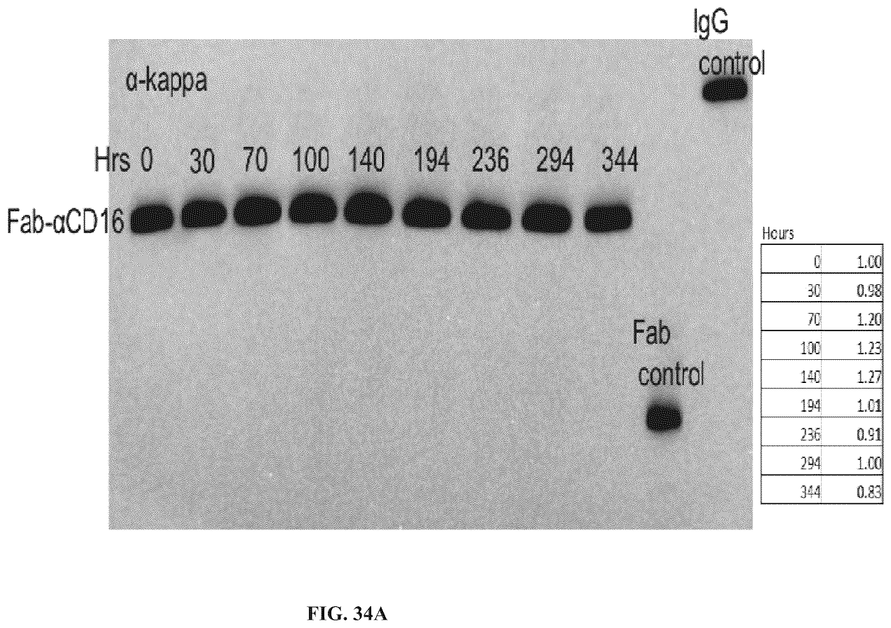

While much of the work above relied on a linear cysteine meditope bearing a diphenylalanine at position 5, Applicants asked whether Applicants could genetically encode a cysteine meditope within a protein and conjugate the protein to meditope-enabled mAbs and mAb fragments bearing the 175Cys modification. First, Applicants tested the conjugation of Eos3.2 fluorescent protein to the cysteine 175Cys Trastuzumab Fab. The DNA sequence encoding the cysteine meditope, SQFDLCTRRLQS (SEQ ID NO:25), was added in-frame to the N-terminus of Eos3.2. Applicants reacted the meditope-Eos3.2 with 175Cys Fab for 16 hours, taking time points throughout. Applicants found the reaction went to completion by four hours (FIG. 32). To avoid the complexity arising from surface cysteines present on Eos3.2, Applicants switched to using moxGFP, a monomeric cysteine free GFP variant. Applicants fused the meditope tag to N- or C-termini of moxGFP to create N-moxGFP (SEQ ID NO:38) and C-moxGFP (SEQ ID NO:39; FIG. 26A). Both constructs readily reacted with 175Cys Fab. In addition, the 175Cys Fab was added to a moxGFP bearing both N- and C-terminal cysteine meditope tags. Purified moxGFP-Fab constructs were verified by SDS PAGE (FIG. 26B). Moreover, the conjugation of the moxGFPs did not affect antigen binding as evidenced by SPR studies (FIG. 26C, FIGS. 33A-33D). Thus, these studies indicate that Applicants can attach protein bearing an N-terminal, C-terminal or both (SEQ ID NO:40) cysteine meditope tag to Fabs to create novel antibody conjugates or create multivalent Fab conjugates (i.e., multiple cys-meditope tags).

Since Applicants could readily attach the GFP variants to the 175Cys trastuzumab Fab and because the interaction stabilizes the Fab, Applicants sought to create bispecific immune engagers ¹⁷. First, the cysteine meditope tag was added to the N—(SEQ ID NO:42) or C—(SEQ ID NO:43) termini of an αCD16 nanobody (FIG. 26D). Both N- and C-terminal meditope αCD16 nanobodies expressed well in E. coli and afforded highly purified material in high yields. The purified, N- and C-terminal αCD16 variants were added to the 175Cys trastuzumab Fab, allowed to react, and purified by chromatography. The formation of the disulfide bond was verified by non-reducing and reducing SDS-PAGE (FIG. 26E).

Next, Applicants tested the efficacy of these constructs in an in vitro, antibody dependent cellular cytotoxicity assay (ADCC). The templated αCD16-Fab construct with the C-terminal fusion demonstrated potent ADCC activity with measured EC50's of 3.2 pM (independent measurement 1) and 4.7 pM (independent measurement 2). The ADCC activity as measured by the in vitro assay was significantly reduced for the N-terminal, templated αCD16-trastuzumab Fab variant. The individual fragments, αCD16 and the trastuzumab meFab, failed to elicit a response. Clinical trastuzumb, 175Cys trastuzumab memAb, and clinical pertuzumab were nearly 100-fold less potent (FIG. 26F).

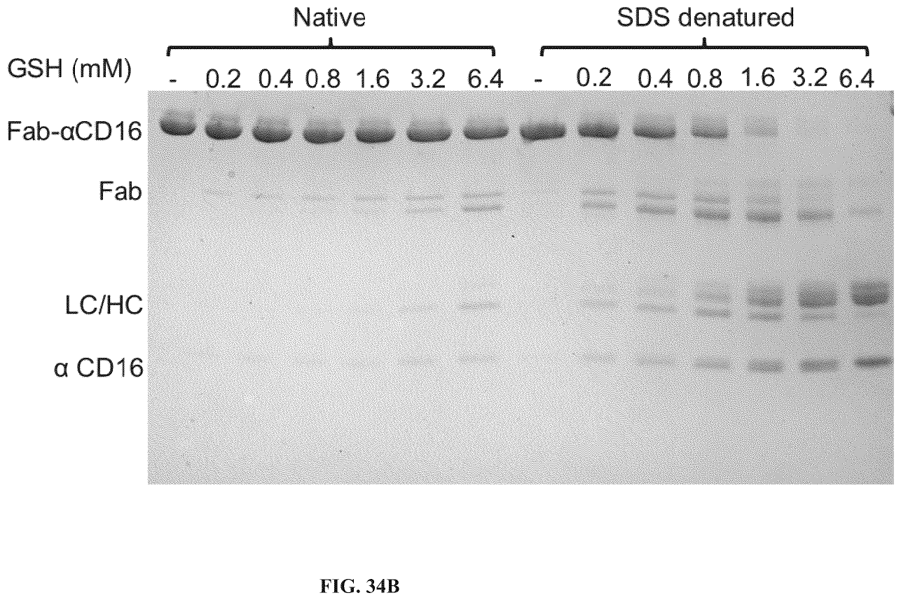

To characterize the serum stability of the templated αCD16-trastuzumab Fab, the bispecific conjugate was incubated in normal rat serum at 37° C. A small aliquot of αCD16-Fab spiked serum was withdrawn over a course of 14 days and immediately frozen. After all the samples were collected, they were separated by SDS-PAGE and detected by Western immunoblotting (FIG. 34A).

Additionally, Applicants tested the stability of the αCD16 Fab by adding increasing amounts of reduced glutathione. The templated complex remains essentially intact up to [GSH]=6.4 mM, much higher than amount of reduced glutathione normally found in the serum, [GSH]ₐᵥₑ=1.02 mM (FIG. 34B)¹⁸.

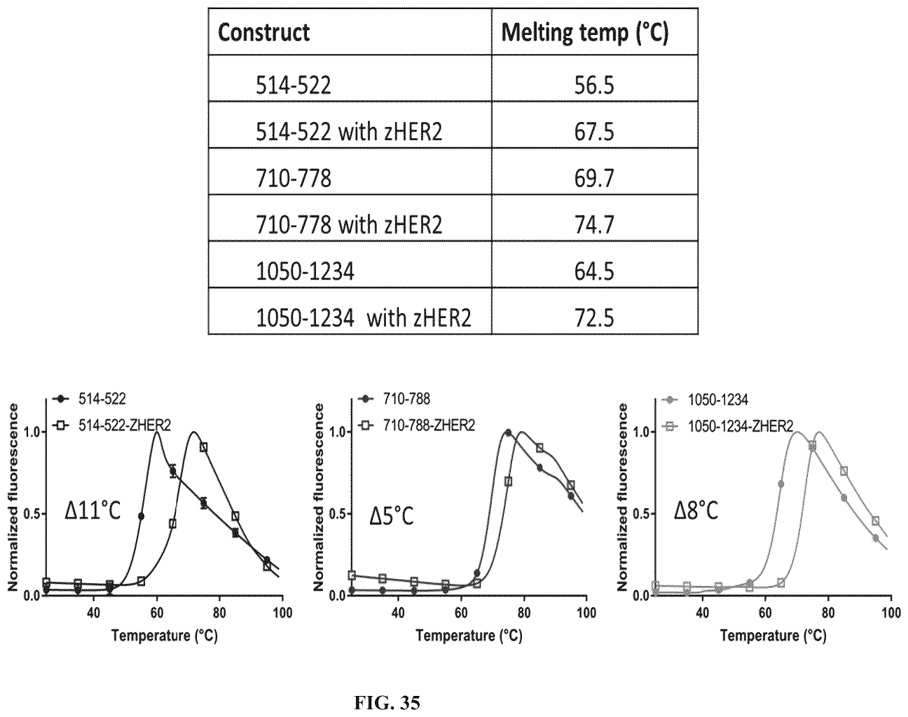

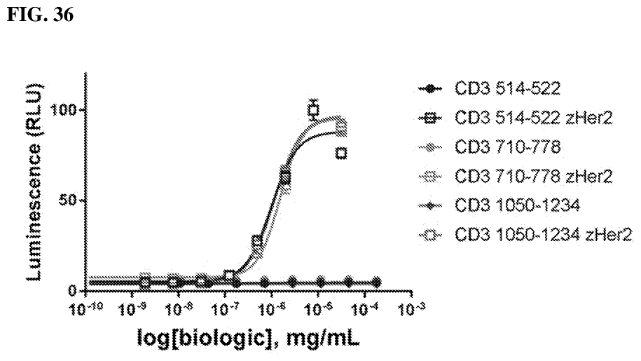

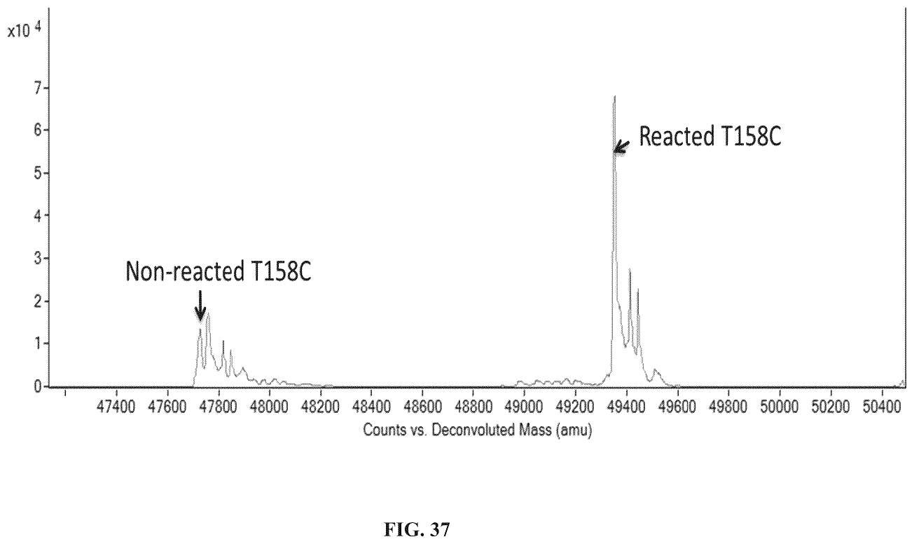

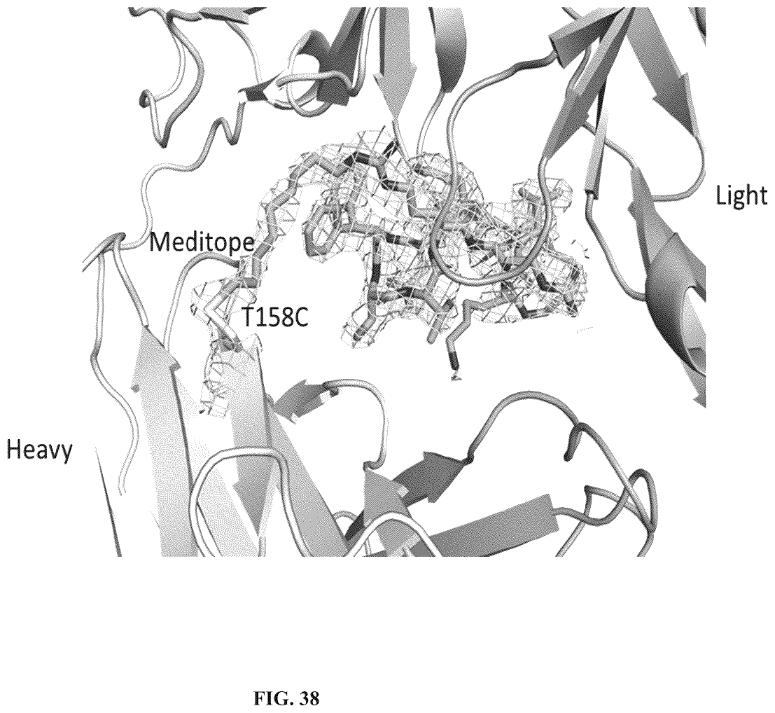

Turning to anti-CD3, Applicants demonstrated the ease and flexibility of this system by creating three different αCD3/HER2 bispecific T-cell engagers (BiTES) (FIG. 26G). Here, genetically encoded a N-terminal cysteine meditope to ZHER2 (SEQ ID NO:41), an affibody that binds human HER2 with high affinity (reported K_{D}=22 pM)¹⁹. Applicants individually mixed the three 175Cys αCD3 Fabs, as described above, with ZHER2 and purified each complex (FIG. 26H). Applicants measured the thermal stability of the αCD3 alone and with ZHER2. The ZHER2 conjugates increased the melting overall temperature by 5-11° C. (FIG. 35). Applicants then tested the ability of these complexes to engage and activate Jurkat cells in the presence of SKBR3 (high Her2 levels) and MCF7 (low Her2 levels) cells. All three Fab-ZHER2 complexes activated T cells whereas the Fab only controls do not. The EC₅₀ of each to the SKBR3 cells is 38, 54 and 34 pM for 514-522, 710-778, and 1050-1234 conjugates, respectively (FIG. 26I). The EC₅₀ of each to the MCF7 cells is 118, 181 and 139, respectively (FIG. 35). Of note, these values differ from the chemically conjugated BiTes (described above). The variation likely reflects differences in the affinity and the geometry of engagement, properties that Applicants will thoroughly explore in future studies.

Taken together, the mPACT platform opens up a combinatoric approach to effortlessly and efficiently create a broad array of stable, antibody conjugates. Using the mPACT platform, Applicants demonstrate that Applicants can recapitulate current methods used to add functionality to mAbs (e.g., chemical conjugation). Distinct from these methods, Applicants also show that Applicants can use natural amino acids to achieve these goals. This feature opens up the possibility to produce potent biologics in other expression systems (e.g., E. coli), which are tolerated to enzymatic activities that are cytotoxic in mammalian cell lines (e.g., diphtheria toxin, PE38; amphibian ribonuclease, onconase; etc). Moreover, unlike many genetic approaches used to create bifunctional molecules, meditope interaction substantially improves the overall stability of the desired bifunctional biologic. In addition, Applicants demonstrate here using three distinct mAbs that grafting the meditope site is straightforward and does not produce a measurable affect on antigen binding in biochemical assays (e.g. SPR), consistent with previous results^{14, 20} Finally, Applicants note that 175Cys meditope-enabled mAbs are made in high yield (frequently over 100 mg/L transiently without recourse to alternation of the expression conditions) and the mPACT'ed reaction is nearly stoichiometric (Table 4). Beyond therapeutic and diagnostic possibilities, Applicants anticipate that the mPACT platform will be useful for basic research including the efficient, site-specific conjugation of mAbs with different fluorophores or biologics (e.g., luciferase).

### Methods

### Protein Production and Purification

Antibodies: Codon optimized DNA for each antibody was produced by ATUM. Fab DNA was obtained by introducing a stop codon after PKSCDKTH sequence. Antibodies were produced by transient expression in ExpiCHO cells (ThermoFisher). Transfection and cell growth were performed following the manufacturer's high titer protocol.

To purify the antibodies, the ExpiCHO medium was centrifuged followed by passage through 0.45 micron and 0.22 micron filters. The clarified medium was then applied to protein G resin (Genscript), rinsed with 20 column volumes of PBS, and eluted with 10 column volumes of 100 mM glycine buffer, pH 3.0. Eluted antibodies were immediately neutralized with 1M Tris pH 9.0. Antibodies were further purified by size exclusion chromatography on an S200 26/60 (GE Healthcare), and stored in PBS at 4° C.

For Fab purification, ExpiCHO medium was clarified as above and the Fabs were purified using protein G resin. Monomeric Fabs were further purified using an S75 26/60 (GE Healthcare).

Bacterially expressed Proteins: Histag-SMT3-ZHER2 and Histag-SMT3-moxGFP fusions were expressed in BL21(DE3) E. coli using Studier's autoiduction media at 25° C. The SMT3 fusions were first purified on 1 mL HisTrap HP (GE Healthcare), cleaved with Histag-ULP1, and subsequently purified to homogentity by reverse nickel chromatography.

The N- and C-teriminal meditope-αCD16 nanobodies were also expressed in BL21(DE3) E. coli using Studier's autoinduction media at 25° C. Each meditope-αCD16 was purified by affinity chromatography using Ni-NTA Superflow (Qiagen) and size-exclusion using a S75 26/60 column (GE Healthcare) in PBS (pH 7.4).

Peptide synthesis: Standard solid-phase N-αFmoc chemistry was used to synthesize the peptides Ac-SQFDA(Ph)₂CTRRLQSGGSK and Ac-SQFDA(Ph)₂STRRLQSGGSK on CS136XT peptide synthesizer (C S BIO). After N-terminal acetylation, the peptide was removed from resin and deprotected using reagent K (TFA/water/phenol/thioanisole/EDT=82.5:5:5:5:2.5). Crude peptide was collected by precipitation from cold ether and purified using a reverse-phase HPLC (Agilent 1200 system with Agilent prep-C18 column, 21.2×150 mm, 5 μm) with a water (0.1% TFA)/acetonitrile (0.1% TFA) solvent system.

For attachment of small molecules to the terminal lysine of Ac-SQFDA(Ph)₂CTRRLQSGGSK, the cysteine was protected with thiopyridine prior to NHS conjugation. Purified peptide was treated with 2,2′-dipyridyl disulfide in 100 mM NaHCO3 buffer and acetonitrile (v:v=2:1) overnight. Thiopyridine modified peptide was isolated by HPLC purification. The thiopyridine modified peptide was then reacted with an NHS ester version of azide (1070-5G, Click Chemistry Tools), tetrazine (1127-25, Click Chemistry Tools), TCO-PEG4 (A137-25, Click Chemistry Tools), DM1, and Glu-Val-Cit-PAB-MMAE (SET0100, Levena Biopharma) in 100 mM NaHCO₃ buffer and acetonitrile (v:v=2:1) for 30 min to provide the corresponding peptides. All peptides were purified using reverse-phase HPLC using appropriate buffer system. All peptides were characterized by mass spectrometry.

The Ac-SQFDFCTRRLQSGGSK peptide was synthesized and purified by CS Bio Co.

Crystallization and structure determination: The Fab of the meTrastuzumab variants were crystallized by hanging drop diffusion as previously described²¹. Equamolar concentrations of protein L and protein A were added to meTrastuzmab Fab (50-80 μM)²¹. The protein A/L/meTrastuzumab Fab complex in crystallization buffer (10 mM NaCl, 1 mM EDTA, 10 mM Tris pH 8.0) was mixed with the precipitant solution (15% PEG 3350, 50 mM Tris, pH 7.5), 1 μL+1 μL. Crystals formed overnight, and were ready for harvesting within 48 hours. Crystals were passed through precipitant solution containing 20% meso-erythritol and flash frozen in liquid nitrogen. Data was collected in house on a Rigaku Micromax X-007 HF with RAXIS IV++ detector or at SSRL beamline 9-2, at 100 K. Data was processed with XDS²², models were built in Coot²³ and structures were refined with Phenix²⁴.

Differential Scanning Fluorimetry (DSF): DSF denaturation curves were used as a proxy for protein stability²⁵. 20 μl DSF reactions consisted of 0.2 mg/mL protein and 5× Sypro Orange (Life Technologies) in 100 mM Hepes (pH 7.4) and 150 mM NaCl. Reactions were carried out in a 384-well plate on a Viia7 real time PCR instrument (Life Technologies) following the manufacturer's protocol. Melting temperature at half-maximal value, Tm, was calculated with Protein Thermal Shift Software v1.3 (Life Technologies).

Surface plasmon resonance binding assays: Surface plasmon resonance was used to determine the affinity of the trastuzumab variants for HER2 on a Biacore T200. HER2-Fc (R&D Systems) was immobilized to a CM5 S-series sensor chip (GE Healthcare) using standard amine coupling (EDC/NHS) in 10 mM acetate buffer pH 4.0 at a density suitable for kinetics. To avoid bivalent binding, Fab was used for all experiments. The Fabs were serially diluted in HBS-EP+(10 mM HEPES pH 7.4, 150 mM NaCl, 3 mM EDTA, 0.05% (v/v) surfactant P20) from 5 nM to 78 pM, and flowed over the chip at 30 μl min⁻¹. The chip was regenerated using 100 mM glycine buffer (pH 2.0) followed by HBS-EP+. Data were processed using Biacore T200 Evaluation software v.3.0.

Liquid Chromatography/Mass Spectrometry: Samples were analyzed using an Agilent 6520 Mass Spectrometer equipped with a HPLC and Chip Cube Source (Agilent, Santa Clara, CA). Samples were separated on a C8 LC/MS chip and sprayed directly into the mass spectrometer. Peaks containing the protein were manually integrated. Spectra from the peak of interest were averaged and a background spectrum from a portion of the gradient during which no protein was eluting was subtracted. Background subtracted spectra were deconvoluted using MassHunter Bioconfirm B06.00 (Agilent) with the Maximum Entropy algorithm.

Conjugation of Alexa647 to 175Cys memAbs: 2 mg of Trastuzumab 175Cys IgG in 500 L of PBS was treated with 20 mM cysteine for 30 minutes at room temperature to reduce the engineered cysteine. Cysteine was removed through >10,000 fold dilution and concentration using 10k Amicon centrifugal filters. AF647 was conjugated to the terminal lysine of thiopyridine protected cysteine meditope (Ac-SQFDA(Ph)₂CTRRLQSGGSK). A ten-fold excess of the AF647 meditope was reacted with the reduced IgG in 500 μL of PBS. The sample was concentrated to 100 microliters using a 10k Amicon centrifugal filter. Unreacted meditope was removed using two rounds of desalting with Zeba 7K MWCO columns (ThermoFisher). The dye-antibody ratio was calculated from the absorbance at 280 nm and 650 nm.

Click chemistry conjugations and reactions: 1 mg of each 175Cys Fab in 500 μL of PBS was reduced with 20 mM cysteine for 30 minutes at room temperature to remove disulfide adducts from the Fab. Cysteine was removed through >10,000 fold dilution and concentration using 10k Amicon centrifugal filters. Five-fold excess of thiopyridine protected cysteine meditopes (Ac-SQFDA(Ph)₂CTRRLQSGGSK) conjugated through their terminal lysines with azide, TCO, or tetrazine were mixed with reduced Fabs in 500 μL of PBS. The reactions were allowed to proceed for four hours, and the excess meditope was removed by 5 rounds of concentrating and diluting with 10k Amicon centrifugal filters (Millipore). The azide meditope was reacted with 175Cys Trastuzumab Fab. After removing the excess meditope, the azide-Fab was incubated with 30k DBCO for 16 hours, and run on an SDS-PAGE gel. The tetrazine and TCO meditopes were reacted with 175Cys Fabs of Trastruzumab and α-CD3 Fabs, respectively. As described above, Applicants removed the excess meditope through ultrafiltration. Applicants incubated the TCO-Fab and tetrazine Fab for 3 hours. The formation of the click was assessed by SDS-PAGE.

DM1 and MMAE conjugation: 1 mg of Trastuzumab 175Cys IgG was reduced with 20 mM cysteine for 30 minutes at room temperature to remove disulfide adducts from the Fab arms. Cysteine was removed through >10,000 fold dilution and concentration using 10k Amicon centrifugal filters. MMAE and DM1 were conjugated to the lysine of the thiopyridine protected cysteine meditopes (Ac-SQFDA(Ph)₂CTRRLQSGGSK). Five-fold excess of the drug-cysteine meditope was dissolved in DMA and mixed with reduced Trastuzumab 175Cys memAb for four hours. The final volume was 800 μL of PBS with 3% DMA. Excess meditope was removed by 5 rounds of concentrating and diluting with PBS containing 3% DMA. The final product was buffer exchanged to pure PBS.

The degree of conjugation was assessed by HPLC using TSKgel Butyl-NPR, 4.6 mm×10 cm column, 2.5 m particle size (Tosoh Biosciences) on Agilent 1200 system. Buffer A consisted of 1.5 M ammonium sulfate, 25 mM phosphate (pH 7.0) and buffer B consisted of 25% isopropanol, 25 mM phosphate (pH 7.0). The sample was run for 60 minutes at 0.5 mL/min from 0-100% buffer B. The antibody-drug ratio for each was calculated by integrating the peak area from the HPLC traces. No free meditope-drug was observed in the HPLC trace, indicating complete removal of the un-reacted meditope-drug.

Cell viability studies for the DM1 and MMAE conjugations: CellTiter-Glo® Luminescent Assay from Promega (#G7571) was used to detect cell viability. BT-474 or SKBR3 were seeded in white-walled 96-well plate and each well contained 100 μl of 10,000 BT-474 cells or 7,000 SKBR3 cells. Seeded cells were incubated at 37° C., 5% CO₂ overnight for adherence. For the treatment, drugs with two fold of final concentration were prepared and 100 μL of drugs were added directly to each well. At 72h, plates were equilibrated at room temperature and CellTiter Glo® Reagent was added to each well for cell lysis and luminescence reaction for 10 min. The luminescence was read by Biotek's Synergy 4 multi-detection microplate reader.

Conjugation of moxGFP to 175Cys Trastuzumab memAbs/Fabs: 175Cys Fab or IgG were independently mixed with N-, C-, or NC-moxGFP. A three-fold excess of N-moxGFP and C-moxGFP were used for reactions with 2 mg of Fab. A four-fold excess of C-moxGFP was used for reacting with 1.5 mg of the IgG. A four-fold excess of Fab was reacted with 2 mg of NC-moxGFP. Each combination was reduced with 20 mM cysteine for 30 minutes at room temperature. Cysteine was removed with 10k Amicon centrifugal filters. The mixtures were incubated for 4 hours at room temperature, and then stored at 4° C. overnight for purification. The conjugates were separated from the starting molecules using a Mono Q GL 5/50 column (GE Healthcare).

Conjugation of ZHER2 to meditope-enabled 175Cys α-CD3 Fabs: 1 mg of each meditope-enabled 175Cys α-CD3 Fab was independently co-reduced with a five-fold excess ZHER2. The Fab-ZHER2 samples were reduced for 30 minutes at room temperature with 20 mM cysteine. Cysteine was removed with 10k Amicon centrifugal filters as before. After removal of cysteine, the mixed samples were allowed to incubate for 4 hours at room temperature. Non-reacted α-CD3 Fab and ZHER2 were separated from the disulfide conjugate using a Mono S GL 5/50 column (GE Healthcare).

T-cell activation assay: ZHER2-α-CD3 Fab conjugates were tested for their ability to activate Jurkat cells expressing luceriferase. SKBR3 cells were seeded at 15,000 cells per 100 μL DMEM per well in 96-well plates and incubated overnight for attachment. The next day, media in 96-well plate were removed and 100,000 Jurkat-Lucia™ NFAT cells (Invivogen #jktl-nfat) per 50 μL RPMI were added in each well. Then, 50 μL of drugs with two fold concentration prepared in RPMI were added in each well containing SKBR3 and Jurkat-Lucia™ NFAT cells. After incubation at 37° C. for 6 h, 50 μL of media from each well were moved to white-walled 96-well plate and 50 μL QUANTI-Luc™ luciferase detection reagents (Invivogen #rep-qlcl) were added in each well. The luminescence was immediately read by Biotek's Synergy 4 multi-detection microplate reader.

Conjugation of α-CD16 nanobody to meditope-enabled 175Cys Trastuzumab Fabs: As with the ZHER2 conjugates, 1 mg of meditope-enabled 175Cys Trastuzumab Fabs was co-reduced with a five-fold excess α-CD16 nanobodies. The Fab-nanobody complexes were reduced for 30 minutes at room temperature, desalted, and allowed to react for 4 hours at room temperature. Non-reacted Fab was removed using 1 mL HiTrap SP HP (GE Healthcare), and non-reacted αCD16 was removed by size-exclusion with a Superdex 75 Increase 10/300 GL (GE Healthcare).

Antibody-dependent cell-mediated cytotoxicity (ADCC) assay: The αCD16-Tras175Cys conjugates were tested for ADCC activity using a commercial ADCC kit (Promega), following their protocol for Trastuzumab. SKBR3 cells were seeded at 5,000 cells per well in the inner 60 wells of a white-walled 96 well plate, and allowed to adhere overnight at 37° C. in 5% CO₂. The following morning the old media was removed. To each well, 25 μL of ADCC assay buffer, 25 μL 3× concentration of diluted antibody, and 25 μL of effector cells were added. The final effector to target cell ratio was 15:1. The mixtures were incubated at 37° C. for 6 hours. The production of luciferase by the target cells were assessed by the addition 75 μl of Bio-Glo™ Luciferase Assay Reagent.

Serum Stability Study: 70 g of purified N-α-CD16-Tras175Cys conjugate was incubated in normal rat serum at 37° C. 2 g aliquots were removed at 0, 30, 70, 100, 140, 194, 236, 294, and 344 hours. Western blotting for the kappa light chain was used to detect the conjugate or Fab alone at each time point. The presence of the Fab alone would indicate the loss of the disulfide bond. The time points were run on a non-reducing 4-20% TGX precast gel (Biorad), and transferred to nitrocellulose by Trans-Blot Turbo system (Biorad). The membrane was blocked for 1 hour with 10% milk in PBST (PBS with 0.05% Tween). The kappa light chain was detected using HRP-anti-Kappa light chain antibody (ab202549, Abcam) at 1/20,000 dilution in PBST. The antibody was incubated for 3 hours at room temperature with the blot, and washed 6 times with PBST. The blot was detected using ECL (Pierce).

Resistance to reduction: 23 M of purified N-α-CD16-Tras175Cys conjugate was incubated at 37° C. for 1 hour with 0, 0.2, 0.4, 0.8, 1.6, 3.2, or 6.4 mM of reduced glutathione in the presence (denatured) or absence of 1% SDS (native). After 1 hour, samples were mixed 1:1 2× Laemmli sample buffer (Biorad), and run on 4-20% TGX precast gel (Biorad). Reduction of the conjugate between N-α-CD16 and the Fab was detected by the appearance of N-α-CD16 or the Fab alone. Reduction of the Fab can be detected by the appearance of the light and heavy chains.

Animal Imaging: Athymic female mice (NCI Charles River), approximately 8 weeks old, received intermuscular (IM) injection of Delestrogen (0.8 mg/0.25 ml, estradiol valerate) two days prior of 5e6 MCF-7.her2 cells suspended in 1% Human Serum Albumin (HSA) in Hanks Balanced Salt Solution (HBSS) total volume 200ul injected subcutaneous at the shoulder or low flank to establish for 21 days.

Mice received 100ug of AF647-175Cys IgG diluted with saline (USP) as a single bolas intravenous injection. The Spectral Instruments Imaging Ami X system, 640 nm excitation and 690 emission filters, was utilized for fluorescent imaging acquisition at 1, 6 and 24 hour post injection time points. Mice were sedated with isoflurane inhalant for imaging acquisition for approximately 20 min. After the 24 hr time point the mice were euthanized and dissected. The tissues were imaged again with same filter sets.

Flow Cytometry: SKBR3 cells were maintained in 10% FBS-supplemented DMEM at 37° C. and 5% CO₂. SKBR3 cells were dislodged using non-enzymatic cell dissociation reagent (C5789, SIGMA), and diluted to a concentration of 1×10⁶ cells per mL. Cells were incubated with 10 nM meTrastuzumab 175Cys, meTrastuzumab 175Cys conjugated with Alexa647 meditope, or clinical trastuzumab in washing buffer (10% FBS in 1× PBS) or washing buffer alone, and incubated on ice for 30 min. Cells were washed three times with washing buffer to remove unbound antibody.

Bound antibodies were detected by the addition of anti-human IgG Fc secondary antibody conjugated to Alexa488 (Life Technologies). Secondary antibody was incubated on ice for 30 minutes. Unbound secondary was removed by three washing steps. Flow cytometry was performed with a BD LSRFortessa cellanalyzer (BD Biosciences), and analyzed using FlowJo software. Fluorescence Imaging: SKBR3 cells were seeded overnight into an 8-well microchamber slide (Ibidi) at a density of 8×10⁴ cells per well (4×10⁵ cells/mL and 0.2 mL per well.) The following day, cells were treated with 50 nM of antibodies in PBS on ice for 1 hour. Cells were washed three times for 5 minutes with PBS and subjected to fixation with 250 μL of 4% paraformaldehyde in PBS for 10 minutes at 37° C. followed by two washes with PBS. Cells were then treated with Alexa 488-labeled anti-human IgG secondary antibody (Thermo Fisher) at a dilution of 1:500 in PBS. After two hours of labeling with the secondary antibody, cells were washed three times for 5 minutes in PBS. Prolong® Gold Antifade Reagent with DAPI (Cell Signaling Technologies) was added to each well and incubated at room temperature overnight prior to imaging at 40× on a Zeiss Axio Observer Z1 inverted microscope (Zeiss). Images were processed and analyzed using ZEN 2.

TABLE 1
Crystallographic data-collection and refinement statistics.

| | | | Trastuzumab | | Trastuzumab | | Trastuzumab | |
|---|---|---|---|---|---|---|---|---|
| | | | I83E, A175C | | I83E, A175C | | I83E, A175C | |
| | Trastuzumab | | SQFDFCTRRLQSG | | SQFDA(Ph)₂CTRR | | AcCQFDLSTRRLK | |
| | I83E, A175C | | GSK | | LQSGGSK | | C(Am) | |
| Data collection | | | | | | | | |
| Space group | P2₁2₁2₁ | | P2₁2₁2₁ | | P2₁2₁2₁ | | P2₁2₁2₁ | |
| Cell dimensions | | | | | | | | |
| a, b, c (Å) | 53.49, 105.29, 117.15 | | 52.99, 104.60, 116.29 | | 53.41, 105.15, 116.98 | | 53.31, 104.94, 117.63 | |
| α, β, γ (°) | 90.0, 90.0, 90.0 | | 90.0, 90.0, 90.0 | | 90.0, 90.0, 90.0 | | 90.0, 90.0, 90.0 | |
| Resolution (Å) | 48.66-1.82 | (1,87-1.82) | 29.90-2.14 | (2.20-2.14) | 33.6-1.90 | (1.95-1.90) | 33.53-2.14 | (2.20-2.14) |
| Rₘₑₐₛ | 0.12 | (2.07) | 0.18 | (1.09) | 0.115 | (1.06) | 0.152 | (0.931) |
| I/σI | 16.8 | (1.9) | 9.1 | (1.7) | 13.2 | (1.7) | 10.3 | (2.1) |
| Completeness (%) | 98.3 | (98.0) | 99.9 | (100.0) | 99.7 | (97.1) | 99.7 | (100) |
| Redundancy | 13.5 | (14.0) | 4.8 | (4.7) | 5.1 | (4.9) | 5.1 | (5.1) |
| Refinement | | | | | | | | |
| Resolution (Å) | 1.82 | | 2.14 | | 1.90 | | 2.14 | |
| No. reflections | 59,086 | | 36,395 | | 52,555 | | 37,064 | |
| R_{work}/R_{free} | 16.5/18.9 | | 17.1/21.8 | | 16.4/19.4 | | 17.3/22.4 | |
| No. atoms | | | | | | | | |
| Protein | 4342 | | 4297 | | 4277 | | 4275 | |
| Ligand | — | | 109 | | 115 | | 104 | |
| Water | 573 | | 535 | | 675 | | 535 | |
| B-factors | | | | | | | | |
| Protein | 36.1 | | 30.0 | | 26.4 | | 28.3 | |
| Ligand | — | | 44.1 | | 43.9 | | 30.3 | |
| Water | 44.1 | | 37.8 | | 38.4 | | 36.9 | |
| R.ms. deviations | | | | | | | | |

| | | | | |
|---|---|---|---|---|
| Bond lengths (Å) | 0.006 | 0.006 | 0.006 | 0.007 |
| Bond angles (°) | 0.840 | 0.865 | 0.854 | 0.872 |

TABLE 2
RMSD values of A175C crystal relative to parental
apo I83E calculated over 434 Cα atoms. The
meditope containing structures have larger RMSD.

| Structure | RMSD |
|---|---|
| Apo A175C | 0.30 Å |
| 5-phenyl A175C | 0.68 Å |
| 5-diphenyl A175C | 0.63 Å |
| Ac-CQFDLSTRRLC-Am | 0.65 Å |

TABLE 3
Biacore values for selected Trastuzumab fab derivatives against
HER2. Error values are the standard deviation of three runs.

| Sample | Kₐ (1/Ms) | K_{d} (1/s) | K_{D} (M) |
|---|---|---|---|
| I83E Fab | 1.04 ± 0.88 × 10⁶ | 6.74 ± 1.69 × 10⁻⁴ | 8.63 ± 3.90 × 10⁻¹⁰ |
| A175C Fab | 1.28 ± 0.03 × 10⁶ | 3.78 ± 0.38 × 10⁻⁴ | 2.96 ± 0.24 × 10⁻¹⁰ |
| A175C Fab w/ | 1.24 ± 0.61 × 10⁶ | 4.19 ± 0.81 × 10⁻⁴ | 4.19 ± 0.42 × 10⁻¹⁰ |
| SQFDA(Ph)₂CTRRLQSGGSK | | | |

TABLE 4
The final drug-antibody ratio does not improve with increasing
the ratio of meditope-DM1 to antibody in the initial reaction.

| Fold drug to | |
|---|---|
| antibody | Final Dar |
| 24x | 1.88 |
| 12x | 1.88 |
| 8x | 1.88 |
| 4x | 1.88 |
| 2x | 1.88 |

### REFERENCES

1. Weiner, G. J. Building better monoclonal antibody-based therapeutics. Nature reviews. Cancer 15, 361-370 (2015).
2. Demarest, S. J. & Glaser, S. M. Antibody therapeutics, antibody engineering, and the merits of protein stability. Current opinion in drug discovery & development 11, 675-687 (2008).
3. Carter, P. J. Introduction to current and future protein therapeutics: a protein engineering perspective. Experimental cell research 317, 1261-1269 (2011).
4. Iyer, U. & Kadambi, V. J. Antibody drug conjugates—Trojan horses in the war on cancer. Journal of pharmacological and toxicological methods 64, 207-212 (2011).
5. Scott, A. M., Allison, J. P. & Wolchok, J. D. Monoclonal antibodies in cancer therapy. Cancer immunity 12, 14 (2012).
6. Junutula, J. R. et al. Site-specific conjugation of a cytotoxic drug to an antibody improves the therapeutic index. Nat Biotechnol 26, 925-932 (2008).
7. Sirk, S. J., Olafsen, T., Barat, B., Bauer, K. B. & Wu, A. M. Site-specific, thiol-mediated conjugation of fluorescent probes to cysteine-modified diabodies targeting CD20 or HER2. Bioconjugate chemistry 19, 2527-2534 (2008).
8. Baumer, N. et al. Antibody-coupled siRNA as an efficient method for in vivo mRNA knockdown. Nat Protoc 11, 22-36 (2016).
9. Witte, M. D. et al. Production of unnaturally linked chimeric proteins using a combination of sortase-catalyzed transpeptidation and click chemistry. Nature protocols 8, 1808-1819 (2013).
10. Wang, L., Amphlett, G., Blattler, W. A., Lambert, J. M. & Zhang, W. Structural characterization of the maytansinoid-monoclonal antibody immunoconjugate, huN901-DM1, by mass spectrometry. Protein Sci 14, 2436-2446 (2005).
11. Junutula, J. R. et al. Rapid identification of reactive cysteine residues for site-specific labeling of antibody-Fabs. Journal of immunological methods 332, 41-52 (2008).
12. Tian, F. et al. A general approach to site-specific antibody drug conjugates. Proc Natl Acad Sci USA 111, 1766-1771 (2014).
13. Klein, C. et al. Cergutuzumab amunaleukin (CEA-IL2v), a CEA-targeted IL-2 variant-based immunocytokine for combination cancer immunotherapy: Overcoming limitations of aldesleukin and conventional IL-2-based immunocytokines. Oncoimmunology 6, e1277306 (2017).
14. Donaldson, J. M. et al. Identification and grafting of a unique peptide-binding site in the Fab framework of monoclonal antibodies. Proceedings of the National Academy of Sciences of the United States ofAmerica 110, 17456-17461 (2013).
15. Zer, C. et al. Engineering a high-affinity peptide binding site into the anti-CEA mAb M5A. Protein engineering, design & selection: PEDS, 1-9 (2017).
16. Choi, H. S. et al. Targeted zwitterionic near-infrared fluorophores for improved optical imaging. Nature biotechnology 31, 148-153 (2013).
17. Brinkmann, U. & Kontermann, R. E. The making of bispecific antibodies. mAbs 9, 182-212 (2017).
18. Richie, J. P., Jr., Skowronski, L., Abraham, P. & Leutzinger, Y. Blood glutathione concentrations in a large-scale human study. Clin Chem 42, 64-70 (1996).
19. Eigenbrot, C., Ultsch, M., Dubnovitsky, A., Abrahmsen, L. & Hard, T. Structural basis for high-affinity HER2 receptor binding by an engineered protein. Proceedings of the National Academy of Sciences of the United States ofAmerica 107, 15039-15044 (2010).
20. Zer, C. et al. Engineering a high-affinity peptide binding site into the anti-CEA mAb M5A. Protein engineering, design & selection: PEDS 30, 409-417 (2017).
21. Donaldson, J. M. et al. Identification and grafting of a unique peptide-binding site in the Fab framework of monoclonal antibodies. Proceedings of the National Academy of Sciences of the United States ofAmerica 110, 17456-17461 (2013).
22. Kabsch, W. XDS. Acta crystallographica. Section D, Biological crystallography 66, 125-132 (2010).
23. Emsley, P. & Cowtan, K. Coot: model-building tools for molecular graphics. Acta crystallographica. Section D, Biological crystallography 60, 2126-2132 (2004).
24. Echols, N. et al. Graphical tools for macromolecular crystallography in PHENIX. Journal of applied crystallography 45, 581-586 (2012).
25. Niesen, F. H., Berglund, H. & Vedadi, M. The use of differential scanning fluorimetry to detect ligand interactions that promote protein stability. Nature protocols 2, 2212-2221 (2007).

### Embodiments

Embodiment 1. A covalent complex comprising:
an antigen binding domain comprising:
   (1) a central hole enclosed by the heavy chain variable (VH) region, the light chain variable (VL) region, the heavy chain constant (CH1) region and the light chain constant (CL) region of said antigen binding domain between a first cavity and a second cavity; and
   (2) a non-CDR peptide binding region comprising:
      (a) said first cavity lined by a first set of amino acid residues of the VH, VL, CH1, and CL regions of said antigen binding domain;
      (b) said second cavity lined by a second set of amino acid residues of the VH, VL, CH1, and CL regions of said antigen binding domain; and
      (c) a hole region enclosing said hole between said first cavity and said second cavity, said hole region lined by a third set of amino acid residues of the VH, VL, CH1, and CL regions of said antigen binding domain;
   wherein said non-CDR peptide binding region comprises a first cysteine; and
   (i) a peptide compound comprising a thiol side chain amino acid covalently bound to said antigen binding domain through a disulfide linkage between said first cysteine and said thiol side chain amino acid.

Embodiment 2. The covalent complex of embodiment 1, wherein said first set of amino acid residues comprises said first cysteine at a position corresponding to Kabat position 102, 142 or 143 of said VL region.

Embodiment 3. The covalent complex of embodiment 1, wherein said second set of amino acid residues comprises said first cysteine at a position corresponding to Kabat position 208 or 158 of said VH region.

Embodiment 4. The covalent complex of embodiment 1, wherein said third set of amino acid residues comprises said first cysteine at a position corresponding to Kabat position 174 or 175 of said VH region.

Embodiment 5. The covalent complex of any one of embodiments 1-4, wherein said non-CDR peptide binding region comprises framework region amino acid residues.

Embodiment 6. The covalent complex of one of embodiments 1-5, wherein said peptide compound has the formula:

R¹-X0-X1-X2-X3-X4-X5-X6-X7-X8-X9-X10-X11-X12-R² (I)
wherein:
   X0 is Ser or null;
   X1 is Ser, Cys, Gly, β-alanine, diaminopropionic acid, β-azidoalanine, or null;
   X2 is Gln or null;
   X3 is Phe, Tyr, β,β′-diphenyl-Ala, His, Asp, 2-bromo-L-phenylalanine, 3-bromo-L-phenylalanine, 4-bromo-L-phenylalanine, Asn, Gln, a modified Phe, a hydratable carbonyl-containing residue, or a boronic acid-containing residue;
   X4 is Asp or Asn;
   X5 is Leu, β,β′-diphenyl-Ala, Phe, Trp, Tyr, a non-natural analog of phenylalanine, tryptophan, or tyrosine, a hydratable carbonyl-containing residue, or a boronic acid-containing residue;
   X6 is said thiol side chain amino acid or serine;
   X7 is the thiol side chain amino acid, Thr, or Ser;
   X8 is said thiol side chain amino acid, Arg, Ala, or an amino acid comprising a side chain of the formula -L^{3A}-L^{3B}-R³, wherein L^{3A} is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, L^{3B} is a chemical linker and R³ is a steric hindering chemical moiety;
   X9 is the thiol side chain amino acid, Arg or Ala;
   X10 is Leu, Gln, Glu, β,β′-diphenyl-Ala, Phe, Trp, Tyr; a non-natural analog of phenylalanine, tryptophan, or tyrosine, a hydratable carbonyl-containing residue, or a boronic acid-containing residue;
   X11 is the thiol side chain amino acid, Gln, Lys or Arg;
   X12 is Ser, Cys, Gly, 7-aminoheptanoic acid, β-alanine, diaminopropionic acid, propargylglycine, isoaspartic acid, or null;
   R¹ is null, -L^{10A}-L^{10B}-R¹⁰, an amino acid peptide sequence optionally substituted with -L^{10A}-L^{10B}-R¹⁰;
   R² is null, -L^{20A}-L^{20B}-R²⁰, an amino acid peptide sequence optionally substituted with -L^{20A}-L^{20B}-R²⁰;
wherein
   L^{10A} L^{10B}, L^{20A}, L^{20B} are independently a bond, a peptidyl linker, —O—, —S—,
   —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; R¹⁰ and R²⁰ are independently a reactive moiety, a diagnostic moiety, a therapeutic moiety or a detectable moiety;
   wherein X1 and X12 are optionally joined together to form a cyclic peptidyl moiety; and
   wherein R¹ and X11 are optionally joined together to form a cyclic peptidyl moiety.

Embodiment 7. The covalent complex of one of embodiments 1-6, wherein R²⁰ is a therapeutic moiety.

Embodiment 8. The covalent complex of one of embodiments 1-7, wherein R²⁰ is a protein moiety.

Embodiment 9. The covalent complex of one of embodiments 1-8, wherein R²⁰ is a nanobody moiety.

Embodiment 10. The covalent complex of one of embodiments 1-9, wherein R²⁰ is a variable heavy chain nanobody moiety.

Embodiment 11. The covalent complex of one of embodiments 1-10, wherein R²⁰ is an anti-CD16 nanobody moiety.

Embodiment 12. The covalent complex of one of embodiments 1-11, wherein L^{20A} or L^{20B} is independently a peptidyl linker.

Embodiment 13. The covalent complex of one of embodiments 1-12, wherein L^{20A} or L^{20B} is independently from about 2 to about 10 amino acids in length.

Embodiment 14. The covalent complex of one of embodiments 1-13, wherein L^{20A} or L^{20B} is independently from about 4 to about 6 amino acids in length.

Embodiment 15. The covalent complex of one of embodiments 1-14, wherein said thiol side chain amino acid at position X6 is Cys.

Embodiment 16. The covalent complex of one of embodiments 1-15, wherein X8 is Arg.

Embodiment 17. The covalent complex of one of embodiments 1-16, wherein X0 is null.

Embodiment 18. The covalent complex of one of embodiments 1-17, wherein X1 and X12 are independently Ser.

Embodiment 19. The covalent complex of one of embodiments 1-17, wherein X1 and X12 are Ser.

Embodiment 20. The covalent complex of one of embodiments 1-19, wherein X5 is β,β′-diphenyl-Ala.

Embodiment 21. The covalent complex of one of embodiments 1-20, wherein R² is a 1 to 100 amino acid peptide sequence.

Embodiment 22. The covalent complex of one of embodiments 1-21, wherein R¹ is null and R² is a 1 to 100 amino acid peptide sequence.

Embodiment 23. The covalent complex of one of embodiments 1-22, wherein R² is -Gly-Gly-Lys.

Embodiment 24. The covalent complex of one of embodiments 1-23, wherein X1 and X12 are optionally joined together to form a cyclic peptidyl moiety.

Embodiment 25. The covalent complex of one of embodiments 1-23, wherein said peptide compound is a linear peptide compound.

Embodiment 26. The covalent complex of one of embodiments 1-25, wherein said peptide compound comprises the sequence of SEQ ID NO:1.

Embodiment 27. The covalent complex of one of embodiments 1-6, wherein X6 is Ser.

Embodiment 28. The covalent complex of one of embodiments 1-6 or 27, wherein X5 is β,β′-diphenyl-Ala.

Embodiment 29. The covalent complex of one of embodiments 1-6 or 27-28, wherein said thiol side chain amino acid at position X8 is a substituted arginine.

Embodiment 30. The covalent complex of one of embodiments 1-6 or 27-29, wherein said substituted arginine is an octyl-thiol-substituted arginine.

Embodiment 31. The covalent complex of one of embodiments 1-6 or 27-30, wherein X0 and X1 are null.

Embodiment 32. The covalent complex of one of embodiments 1-6 or 27-31, wherein X11 is lysine.

Embodiment 33. The covalent complex of one of embodiments 1-6 or 27-32, wherein R² is a 1 to 100 amino acid peptide sequence.

Embodiment 34. The covalent complex of one of embodiments 1-6 or 27-33, wherein R¹ is null and R² is a 1 to 100 amino acid peptide sequence.

Embodiment 35. The covalent complex of one of embodiments 1-6 or 27-34, wherein R² is -Gly-Gly-Lys.

Embodiment 36. The covalent complex of one of embodiments 1-6 or 27-32, wherein R¹ and X11 are optionally joined together to form a cyclic peptidyl moiety.

Embodiment 37. The covalent complex of one of embodiments 1-6 or 27-36, wherein said peptide compound comprises the sequence of SEQ ID NO:2.

Embodiment 38. The covalent complex of one of embodiments 1-6, wherein X6 is Ser.

Embodiment 39. The covalent complex of one of embodiments 1-6 or 38, wherein X5 is β,β′-diphenyl-Ala.

Embodiment 40. The covalent complex of one of embodiments 1-6 or 38-39, wherein X8 is Arg.

Embodiment 41. The covalent complex of one of embodiments 1-6 or 38-40, wherein X12 is Ser.

Embodiment 42. The covalent complex of one of embodiments 1-6 or 38-41, wherein R² is a 1 to 100 amino acid peptide sequence.

Embodiment 43. The covalent complex of one of embodiments 1-6 or 38-42, wherein R¹ is null and R² is a 1 to 100 amino acid peptide sequence.

Embodiment 44. The covalent complex of one of embodiments 1-6 or 38-43, wherein R² is -Ser-Gly-X15-Gly-Lys, wherein X15 is said thiol side chain amino acid.

Embodiment 45. The covalent complex of embodiment 44, wherein X15 is Cys.

Embodiment 46. The covalent complex of one of embodiments 1-6 or 38-44, wherein X1 and X12 are optionally joined together to form a cyclic peptidyl moiety.

Embodiment 47. The covalent complex of one of embodiments 1-6 or 38-46, wherein said peptide compound comprises the sequence of SEQ ID NO:3.

Embodiment 48. The covalent complex of one of embodiments 1-5, wherein said peptide compound has the formula:

R¹-X0-X1-X2-X3-X4-X5-X6-X7-X8-X9-X10-X11-X12-X13-X14-X15-R²(II)
wherein:
   X0 is Ser or null;
   X1 is Ser, Cys, Gly, β-alanine, diaminopropionic acid, β-azidoalanine, or null;
   X2 is Gln or null;
   X3 is Phe, Tyr, β,β′-diphenyl-Ala, His, Asp, 2-bromo-L-phenylalanine, 3-bromo-L-phenylalanine, 4-bromo-L-phenylalanine, Asn, Gln, a modified Phe, a hydratable carbonyl-containing residue, or a boronic acid-containing residue;
   X4 is Asp or Asn;
   X5 is Leu, β,β′-diphenyl-Ala, Phe, Trp, Tyr, a non-natural analog of phenylalanine, tryptophan, or tyrosine, a hydratable carbonyl-containing residue, or a boronic acid-containing residue;
   X6 is Ser;
   X7 is the thiol side chain amino acid, Thr, or Ser;
   X8 is Arg, Ala, or an amino acid comprising a side chain of the formula -L^{3A}-L^{3B}-R³, wherein L^{3A} is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, L^{3B} is a chemical linker and R³ is a steric hindering chemical moiety;
   X9 is the thiol side chain amino acid, Arg or Ala;
   X10 is Leu, Gln, Glu, β,β′-diphenyl-Ala, Phe, Trp, Tyr; a non-natural analog of phenylalanine, tryptophan, or tyrosine, a hydratable carbonyl-containing residue, or a boronic acid-containing residue;
   X11 is the thiol side chain amino acid, Gln, Lys or Arg;
   X12 is Ser, Cys, Gly, 7-aminoheptanoic acid, β-alanine, diaminopropionic acid, propargylglycine, isoaspartic acid, or null;
   X13 is Gly or Ser;
   X14 and X15 are independently Gly, Ser, Ala or said thiol side chain amino acid;
   R¹ is null, -L^{10A}-L^{10B}-R₁₀, an amino acid peptide sequence optionally substituted with -L^{10A}-L^{10B}-R¹;
   R² is null, -L^{20A}-L^{20B}-R²⁰, an amino acid peptide sequence optionally substituted with -L^{20A}-L^{20B}-R²⁰;
wherein
   L^{10A}, L^{10B}, L^{20A}, L^{20B} are independently a bond, a peptidyl linker, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
   R¹⁰ and R²⁰ are independently a reactive moiety, a diagnostic moiety, a therapeutic moiety or a detectable moiety; and
   wherein X1 and X12 are optionally joined together to form a cyclic peptidyl moiety.

Embodiment 49. The covalent complex of embodiment 48, wherein R²⁰ is a therapeutic moiety.

Embodiment 50. The covalent complex of one of embodiments 48-49, wherein R²⁰ is a protein moiety.

Embodiment 51. The covalent complex of one of embodiments 48-50, wherein R²⁰ is a nanobody moiety.

Embodiment 52. The covalent complex of one of embodiments 48-51, wherein R²⁰ is a variable heavy chain nanobody moiety.

Embodiment 53. The covalent complex of one of embodiments 48-52, wherein R²⁰ is an anti-CD16 nanobody moiety.

Embodiment 54. The covalent complex of one of embodiments 48-53, wherein L^{20A} or L^{20B} is independently a peptidyl linker.

Embodiment 55. The covalent complex of one of embodiments 48-54, wherein L^{20A} or L^{20B} is independently from about 2 to about 10 amino acids in length.

Embodiment 56. The covalent complex of one of embodiments 48-55, wherein L^{20A} or L^{20B} is independently from about 4 to about 6 amino acids in length.

Embodiment 57. The covalent complex of one of embodiments 48-56, wherein X15 is said thiol side chain amino acid.

Embodiment 58. The covalent complex of embodiment 48, wherein R² is a 1 to 100 amino acid peptide sequence.

Embodiment 59. The covalent complex of embodiment 48, wherein R¹ is null and R² is a 1 to 100 amino acid peptide sequence.

Embodiment 60. The covalent complex of embodiment 48, wherein R² is -Gly-Lys.

Embodiment 61. The covalent complex of one of embodiments 48-60, wherein said peptide compound comprises the sequence of SEQ ID NO:3.

Embodiment 62. The covalent complex of one of embodiments 1-61, wherein said antigen binding domain comprises a fragment antigen-binding (Fab) domain.

Embodiment 63. The covalent complex of one of embodiments 1-62, wherein said antigen binding domain comprises an Fc domain.

Embodiment 64. The covalent complex of one of embodiments 1-61, wherein said antigen binding domain is a fragment antigen-binding (Fab) domain.

Embodiment 65. The covalent complex of one of embodiments 1-64, wherein said antigen binding domain is a humanized antigen binding domain.

Embodiment 66. The covalent complex of one of embodiments 1-65, wherein said non-CDR peptide binding region is formed by amino acid residues at positions 8, 9, 10, 38, 39, 40, 41, 42, 43, 44, 45, 82, 83, 84, 85, 86, 87, 99, 100, 101, 102, 103, 104, 105, 142, 162, 163, 164, 165, 166, 167, 168, and 173 of said VL region and 6, 9, 38, 39, 40, 41, 42, 43, 44, 45, 84, 86, 87, 88, 89, 90, 91, 103, 104, 105, 106, 107, 108, 111, 110, 147, 150, 151, 152, 173, 174, 175, 176, 177, 185, 186, and 187 of said VH region, according to Kabat numbering.

Embodiment 67. The covalent complex of one of embodiments 1-66, wherein said non-CDR peptide binding region comprises a Glu at position 83 of said VL region, according to Kabat numbering.

Embodiment 68. The covalent complex of one of embodiments 1-67, wherein said non-CDR peptide binding region comprises a Thr or Ser at position 40 of said VH region, according to Kabat numbering.

Embodiment 69. The covalent complex of one of embodiments 1-68, wherein said non-CDR peptide binding region comprises an Asn at position 41 of said VL region, according to Kabat numbering.

Embodiment 70. The covalent complex of one of embodiments 1-69, wherein said non-CDR peptide binding region comprises an Asp or Asn at position 85 of said VL region, according to Kabat numbering.

Embodiment 71. The covalent complex of one of embodiments 1-70, wherein said antigen binding domain binds to an antigen with increased affinity relative to the absence of said peptide compound.

Embodiment 72. The covalent complex of one of embodiments 1-71, wherein said antigen binding domain binds to an antigen with a K_{D} of less than 100 nM.

Embodiment 73. The covalent complex of one of embodiments 1-72, wherein said antigen binding domain binds to an antigen with a K_{D} of less than 50 nM.

Embodiment 74. The covalent complex of one of embodiments 1-73, wherein said antigen binding domain binds to an antigen with a K_{D} of less than 10 nM.

Embodiment 75. The covalent complex of one of embodiments 1-74, wherein said antigen binding domain binds to an antigen with a K_{D} of less than 1 nM.

Embodiment 76. A peptide compound of formula:

R¹-X0-X1-X2-X3-X4-X5-X6-X7-X8-X9-X10-X11-X12-R² (I)
wherein:
   X0 is Ser or null;
   X1 is Ser, Cys, Gly, β-alanine, diaminopropionic acid, β-azidoalanine, or null;
   X2 is Gln or null;
   X3 is Phe, Tyr, β,β′-diphenyl-Ala, His, Asp, 2-bromo-L-phenylalanine, 3-bromo-L-phenylalanine, 4-bromo-L-phenylalanine, Asn, Gln, a modified Phe, a hydratable carbonyl-containing residue, or a boronic acid-containing residue;
   X4 is Asp or Asn;
   X5 is Leu, β,β′-diphenyl-Ala, Phe, Trp, Tyr, a non-natural analog of phenylalanine, tryptophan, or tyrosine, a hydratable carbonyl-containing residue, or a boronic acid-containing residue;
   X6 is Cys, protected Cys or Ser;
   X7 is Cys, protected Cys, Thr, or Ser;
   X8 is protected Arg, Arg, Ala, or an amino acid comprising a side chain of the formula -L^{3A}-L^{3B}-R³, wherein L^{3A} is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, L^{3B} is a chemical linker and R³ is a steric hindering chemical moiety;
   X9 is Cys, protected Cys, Arg or Ala;
   X10 is Leu, Gln, Glu, β,β′-diphenyl-Ala, Phe, Trp, Tyr; a non-natural analog of phenylalanine, tryptophan, or tyrosine, a hydratable carbonyl-containing residue, or a boronic acid-containing residue;
   X11 is Cys, protected Cys, Gln, Lys or Arg;
   X12 is Ser, Cys, protected Cys, Gly, 7-aminoheptanoic acid, β-alanine, diaminopropionic acid, propargylglycine, isoaspartic acid, or null;
   R¹ is null, -L^{10A}-L^{10B}-R¹⁰, an amino acid peptide sequence optionally substituted with -L^{10A}-L^{10B}-R¹⁰;
   R² is null, -L^{20A}-L^{20B}-R²⁰, an amino acid peptide sequence optionally substituted with -L^{20A}-L^{20B}-R²⁰;
wherein
   L^{10A} L^{10B}, L^{20A}, L^{20B} are independently a bond, a peptidyl linker, —O—, —S—,
   —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
   R¹⁰ and R²⁰ are independently a reactive moiety, a diagnostic moiety, a therapeutic moiety or a detectable moiety;
   wherein X1 and X12 are optionally joined together to form a cyclic peptidyl moiety; and
   wherein R¹ and X11 are optionally joined together to form a cyclic peptidyl moiety.

Embodiment 77. The compound of embodiment 76, wherein R²⁰ is a therapeutic moiety.

Embodiment 78. The compound of one of embodiments 76-77, wherein R²⁰ is a protein moiety.

Embodiment 79. The compound of one of embodiments 76-78, wherein R²⁰ is a nanobody moiety.

Embodiment 80. The compound of one of embodiments 76-79, wherein R²⁰ is a variable heavy chain nanobody moiety.

Embodiment 81. The compound of one of embodiments 76-80, wherein R²⁰ is an anti-CD16 nanobody moiety.

Embodiment 82. The compound of one of embodiments 76-81, wherein L^{20A} or L^{20B} is independently a peptidyl linker.

Embodiment 83. The compound of one of embodiments 76-82, wherein L^{20A} or L^{20B} is independently from about 2 to about 10 amino acids in length.

Embodiment 84. The compound of one of embodiments 76-83, wherein L^{20A} or L^{20B} is independently from about 4 to about 6 amino acids in length.

Embodiment 85. The compound of one of embodiments 76-84, wherein X6 is Cys.

Embodiment 86. The compound of one of embodiments 76-85, wherein X6 is protected Cys.

Embodiment 87. The compound of any one of embodiments 76-86, wherein X8 is Arg.

Embodiment 88. The compound of any one of embodiments 76-87, wherein X0 is null.

Embodiment 89. The compound of any one of embodiments 76-88, wherein X1 and X12 are independently Ser.

Embodiment 90. The compound of any one of embodiments 76-88, wherein X1 and X12 are Ser.

Embodiment 91. The compound of any one of embodiments 76-90, wherein X5 is β,β′-diphenyl-Ala.

Embodiment 92. The compound of any one of embodiments 76-91, wherein R¹ is null and R² is a 1 to 100 amino acid peptide sequence.

Embodiment 93. The compound of any one of embodiments 76-92, wherein R² is a 1 to 100 amino acid peptide sequence.

Embodiment 94. The compound of any one of embodiments 76-93, wherein R² is -Gly-Gly-Lys.

Embodiment 95. The compound of any one of embodiments 76-94, wherein X1 and X12 are optionally joined together to form a cyclic peptidyl moiety.

Embodiment 96. The compound of any one of embodiments 76-95, wherein said peptide compound is a linear peptide compound. wherein said peptide compound comprises the sequence of SEQ ID NO:1.

Embodiment 97. The compound of embodiment 76, wherein X6 is Ser.

Embodiment 98. The compound of embodiment 76, wherein X5 is β,β′-diphenyl-Ala.

Embodiment 99. The compound of embodiment 76 or 98, wherein X8 is Arg.

Embodiment 100. The compound of any one of embodiments 76 or 98-99, wherein X12 is Ser.

Embodiment 101. The compound of any one of embodiments 76 or 98-100, wherein R² is a 1 to 100 amino acid peptide sequence.

Embodiment 102. The compound of any one of embodiments 76 or 98-101, wherein R¹ is null and R² is a 1 to 100 amino acid peptide sequence.

Embodiment 103. The compound of any one of embodiments 76 or 98-102, wherein R² is -Ser-Gly-X15-Gly-Lys, wherein X15 is Cys or protected Cys.

Embodiment 104. The compound of embodiment 103, wherein X15 is Cys.

Embodiment 105. The compound of embodiment 103, wherein X15 is protected Cys.

Embodiment 106. The compound of any one of embodiments 76 or 98-105, wherein X1 and X12 are optionally joined together to form a cyclic peptidyl moiety.

Embodiment 107. The compound of any one of embodiments 76 or 98-106, wherein said peptide compound comprises the sequence of SEQ ID NO:3.

Embodiment 108. A peptide compound of formula:

R¹-X0-X1-X2-X3-X4-X5-X6-X7-X8-X9-X10-X11-X12-X13-X14-X15-R² (II)
wherein:
   X0 is Ser or null;
   X1 is Ser, Cys, Gly, β-alanine, diaminopropionic acid, β-azidoalanine, or null;
   X2 is Gln or null;
   X3 is Phe, Tyr, β,β′-diphenyl-Ala, His, Asp, 2-bromo-L-phenylalanine, 3-bromo-L-phenylalanine, 4-bromo-L-phenylalanine, Asn, Gln, a modified Phe, a hydratable carbonyl-containing residue, or a boronic acid-containing residue;
   X4 is Asp or Asn;
   X5 is Leu, β,β′-diphenyl-Ala, Phe, Trp, Tyr, a non-natural analog of phenylalanine, tryptophan, or tyrosine, a hydratable carbonyl-containing residue, or a boronic acid-containing residue;
   X6 is Ser;
   X7 is Cys, protected Cys, Thr, or Ser;
   X8 is Arg, Ala, or an amino acid comprising a side chain of the formula -L^{3A}-L^{3B}-R³, wherein L^{3A} is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, L^{3B} is a chemical linker and R³ is a steric hindering chemical moiety;
   X9 is Cys, protected Cys, Arg or Ala;
   X10 is Leu, Gln, Glu, β,β′-diphenyl-Ala, Phe, Trp, Tyr; a non-natural analog of phenylalanine, tryptophan, or tyrosine, a hydratable carbonyl-containing residue, or a boronic acid-containing residue;
   X11 is Cys, protected Cys, Gln, Lys or Arg;
   X12 is Ser, Cys, protected Cys, Gly, 7-aminoheptanoic acid, β-alanine, diaminopropionic acid, propargylglycine, isoaspartic acid, or null;
   X13 is Gly or Ser;
   X14 and X15 are independently Gly, Ser, Ala, Cys or protected Cys;
   R¹ is null, -L^{10A}-L^{10B}-R¹⁰, an amino acid peptide sequence optionally substituted with -L^{10A}-L^{10B}-R¹⁰;
   R² is null, -L^{20A}-L^{20B}-R²⁰, an amino acid peptide sequence optionally substituted with -L^{20A}-L^{20B}-R²⁰;
wherein
   L^{10A} L^{10B}, L^{20A}, L^{20B} are independently a bond, a peptidyl linker, —O—, —S—,
   —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
   R¹⁰ and R²⁰ are independently a reactive moiety, a diagnostic moiety, a therapeutic moiety or a detectable moiety; and
   wherein X1 and X12 are optionally joined together to form a cyclic peptidyl moiety.

Embodiment 109. The compound of embodiment 108, wherein R²⁰ is a therapeutic moiety.

Embodiment 110. The compound of one of embodiments 108-109, wherein R²⁰ is a protein moiety.

Embodiment 111. The compound of one of embodiments 108-110, wherein R²⁰ is a nanobody moiety.

Embodiment 112. The compound of one of embodiments 108-111, wherein R²⁰ is a variable heavy chain nanobody moiety.

Embodiment 113. The compound of one of embodiments 108-112, wherein R²⁰ is an anti-CD16 nanobody moiety.

Embodiment 114. The compound of one of embodiments 108-113, wherein L^{20A} or L^{20B} is independently a peptidyl linker.

Embodiment 115. The compound of one of embodiments 108-114, wherein L^{20A} or L^{20B} is independently from about 2 to about 10 amino acids in length.

Embodiment 116. The compound of one of embodiments 108-115, wherein L^{20A} or L^{20B} is independently from about 4 to about 6 amino acids in length.

Embodiment 117. The compound of one of embodiments 108-116, wherein X15 is Cys.

Embodiment 118. The compound of one of embodiments 108-117, wherein X15 is protected Cys.

Embodiment 119. The compound of embodiment 117 or 118, wherein R² is a 1 to 100 amino acid peptide sequence.

Embodiment 120. The compound any one of embodiments 108-119, wherein R¹ is null and R² is a 1 to 100 amino acid peptide sequence.

Embodiment 121. The compound of any one of embodiments 108-120, wherein R² is -Gly-Lys.

Embodiment 122. The compound of any one of embodiments 108-121, wherein said peptide compound comprises the sequence of SEQ ID NO:3.

Embodiment 123. An antigen binding domain comprising:
(1) a central hole enclosed by the heavy chain variable (VH) region, the light chain variable (VL) region, the heavy chain constant (CH1) region and the light chain constant (CL) region of said antigen binding domain between a first cavity and a second cavity; and
(2) a non-CDR peptide binding region comprising:
   (a) said first cavity lined by a first set of amino acid residues of the VH, VL, CH1, and CL regions of said antigen binding domain, wherein said first set of amino acid residues comprises a cysteine at a position corresponding to Kabat position 102, 142 or 143 of said VL region;
   (b) said second cavity lined by a second set of amino acid residues of the VH, VL, CH1, and CL regions of said antigen binding domain, wherein said second set of amino acid residues comprises a cysteine at a position corresponding to Kabat position 208 or 158 of said VH region; or
   (c) a hole region enclosing said hole between said first cavity and said second cavity, said hole region lined by a third set of amino acid residues of the VH, VL, CH1, and CL regions of said antigen binding domain, wherein said third set of amino acid residues comprises a cysteine at a position corresponding to Kabat position 174 or 175 of said VH region.

Embodiment 124. The antigen binding domain of embodiment 123, wherein said antigen binding domain comprises a fragment antigen-binding (Fab) domain.

Embodiment 125. The antigen binding domain of embodiment 123 or 124, wherein said antigen binding domain comprises an Fc domain.

Embodiment 126. The antigen binding domain of embodiment 123, wherein said antigen binding domain is a fragment antigen-binding (Fab) domain.

Embodiment 127. The antigen binding domain of one of embodiments 123-126, wherein said antigen binding domain is a humanized antigen binding domain.

Embodiment 128. The antigen binding domain of one of embodiments 123-127, wherein said non-CDR peptide binding region comprises framework region amino acid residues.

Embodiment 129. The antigen binding domain of one of embodiments 123-128, wherein said non-CDR peptide binding region is formed by amino acid residues at positions 8, 9, 10, 38, 39, 40, 4142, 43, 44, 45, 82, 83, 84, 85, 86, 87, 99, 100, 101, 102, 103, 104, 105, 142, 162, 163, 164, 165, 166, 167, 168, and 173 of said VL region and 6, 9, 38, 39, 40, 41, 42, 43, 44, 45, 84, 86, 87, 88, 89, 90, 91, 103, 104, 105, 106, 107, 108, 111, 110, 147, 150, 151, 152, 173, 174, 175, 176, 177, 185, 186, and 187 of said VH region, according to Kabat numbering.

Embodiment 130. The antigen binding domain of one of embodiments 123-129, wherein said non-CDR peptide binding region comprises a Glu at position 83 of said VL region, according to Kabat numbering.

Embodiment 131. The antigen binding domain of one of embodiments 123-130, wherein said non-CDR peptide binding region comprises a Thr or Ser at position 40 of said VH region, according to Kabat numbering.

Embodiment 132. The antigen binding domain of one of embodiments 123-131, wherein said non-CDR peptide binding region comprises an Asn at position 41 of said VL region, according to Kabat numbering.

Embodiment 133. The antigen binding domain of one of embodiments 123-132, wherein said non-CDR peptide binding region comprises an Asp or Asn at position 85 of said VL region, according to Kabat numbering.

1. A covalent complex comprising:
(i) an antibody or fragment antigen-binding (Fab) comprising:
(1) a central hole enclosed by the heavy chain variable (VH) region, the light chain variable (VL) region, the heavy chain constant (CH1) region and the light chain constant (CL) region of said antibody or fragment antigen-binding (Fab) between a first cavity and a second cavity; and
(2) a non-CDR peptide binding region comprising:
(a) said first cavity lined by a first set of amino acid residues of the VH, VL, CH1, and CL regions of said antibody or fragment antigen-binding (Fab);
(b) said second cavity lined by a second set of amino acid residues of the VH, VL, CH1, and CL regions of said antibody or fragment antigen-binding (Fab); and
(c) a hole region enclosing said hole between said first cavity and said second cavity, said hole region lined by a third set of amino acid residues of the VH, VL, CH1, and CL regions of said antibody or fragment antigen-binding (Fab);
wherein said non-CDR peptide binding region comprises a first cysteine; and
(ii) a peptide compound comprising a thiol side chain amino acid covalently bound to said antibody or fragment antigen-binding (Fab) through a disulfide linkage between said first cysteine and said thiol side chain amino acidi wherein said peptide compound has the formula:
R¹-X0-X1-X2-X3-X4-X5-X6-X7-X8-X9-X10-X11-X12-R² (I)
wherein:
X0 is Ser or null;
X1 is Ser, Cys, Gly, β-alanine, diaminopropionic acid, β-azidoalanine, or null;
X2 is Gln or null;
X3 is Phe, Tyr, 0,0′-diphenyl-Ala, His, Asp, 2-bromo-L-phenylalanine, 3-bromo-L-phenylalanine, 4-bromo-L-phenylalanine, Asn, Gln, a modified Phe, a hydratable carbonyl-containing residue, or a boronic acid-containing residue;
X4 is Asp or Asn;
X5 is Leu, 0,0′-diphenyl-Ala, Phe, Trp, Tyr, a non-natural analog of phenylalanine, tryptophan, or tyrosine, a hydratable carbonyl-containing residue, or a boronic acid-containing residue;
X6 is said thiol side chain amino acid or serine;
X7 is the thiol side chain amino acid, Thr, or Ser;
X8 is said thiol side chain amino acid, Arm, Ala, or an amino acid comprising a side chain of the formula -L^{3A}-L^{3B}-R³, wherein L^{3A} is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, L^{3B} is a chemical linker and R³ is a steric hindering chemical moiety;
X9 is the thiol side chain amino acid, Arg or Ala;
X10 is Leu, Gln, Glu, 0,0′-diphenyl-Ala, Phe, Trp, Tyr; a non-natural analog of phenylalanine, tryptophan, or tyrosine, a hydratable carbonyl-containing residue, or a boronic acid-containing residue;
X11 is the thiol side chain amino acid, Gln, Lys or Arg;
X12 is Ser, Cys, Gly, 7-aminoheptanoic acid, 0-alanine, diaminopropionic acid, propargylglycine, isoaspartic acid, or null;
R¹ is null, -L^{10A}-L^{10B}-R¹⁰, an amino acid peptide sequence optionally substituted with -L^{10A}-L^{10B}-R¹⁰;
R² is null, -L^{20A}-L^{20B}-R²⁰, an amino acid peptide sequence optionally substituted with -L^{20A}-L^{20B}-R²⁰;
wherein
L^{10A}, L^{10B}, L^{20A}, L^{20B} are independently a bond, a peptidyl linker, —O—, —S—,
—C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
R¹⁰ and R²⁰ are independently a reactive moiety, a diagnostic moiety, a therapeutic moiety or a detectable moiety;
wherein X1 and X12 are optionally joined together to form a cyclic peptidyl moiety; and
wherein R¹ and X11 are optionally joined together to form a cyclic peptidyl moiety.

2. The covalent complex of claim 1, wherein said second set of amino acid residues comprises said first cysteine at a position corresponding to Kabat position 158 of said VH region.

3. The covalent complex of claim 1, wherein said third set of amino acid residues comprises said first cysteine at a position corresponding to Kabat position 174 or 175 of said VH region.

4.-5. (canceled)

6. The covalent complex of claim 1, wherein R²⁰ is a therapeutic moiety.

7.-10. (canceled)

11. The covalent complex of claim 1, wherein L^{20A} or L^{20B} is independently a peptidyl linker.

12.-13. (canceled)

14. The covalent complex of claim 1, wherein said thiol side chain amino acid at position X6 is Cys.

15. The covalent complex of claim 1, wherein X8 is Arg.

16.-24. (canceled)

25. The covalent complex of claim 1, wherein said peptide compound comprises the sequence of SEQ ID NO:1.

26. The covalent complex of claim 1, wherein X6 is Ser.

27. (canceled)

28. The covalent complex of claim 26, wherein said thiol side chain amino acid at position X8 is a substituted arginine.

29. The covalent complex of claim 28, wherein said substituted arginine is an octyl-thiol-substituted arginine.

30.-35. (canceled)

36. The covalent complex of claim 26, wherein said peptide compound comprises the sequence of SEQ ID NO:2.

37. (canceled)

38. The covalent complex of claim 1, wherein X5 is β,β′-diphenyl-Ala.

39. The covalent complex of claim 38, wherein X8 is Arg.

40.-42. (canceled)

43. The covalent complex of claim 40, wherein R² is -Ser-Gly-X15-Gly-Lys, wherein X15 is said thiol side chain amino acid.

44. The covalent complex of claim 43, wherein X15 is Cys.

45. (canceled)

46. The covalent complex of claim 44, wherein said peptide compound comprises the sequence of SEQ ID NO:3.

47.-74. (canceled)

75. A peptide compound of formula:
R¹-X0-X1-X2-X3-X4-X5-X6-X7-X8-X9-X10-X11-X12-R² (I)
wherein:
X0 is Ser or null;
X1 is Ser, Cys, Gly, β-alanine, diaminopropionic acid, β-azidoalanine, or null;
X2 is Gln or null;
X3 is Phe, Tyr, β,β′-diphenyl-Ala, His, Asp, 2-bromo-L-phenylalanine, 3-bromo-L-phenylalanine, 4-bromo-L-phenylalanine, Asn, Gln, a modified Phe, a hydratable carbonyl-containing residue, or a boronic acid-containing residue;
X4 is Asp or Asn;
X5 is Leu, β,β′-diphenyl-Ala, Phe, Trp, Tyr, a non-natural analog of phenylalanine, tryptophan, or tyrosine, a hydratable carbonyl-containing residue, or a boronic acid-containing residue;
X6 is Cys or Ser;
X7 is Cys, Thr, or Ser;
X8 is protected Arg, Arg, Ala, or an amino acid comprising a side chain of the formula -L^{3A}-L^{3B}-R³, wherein L^{3A} is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, L^{3B} is a chemical linker and R³ is a steric hindering chemical moiety;
X9 is Cys, Arg or Ala;
X10 is Leu, Gln, Glu, β,β′-diphenyl-Ala, Phe, Trp, Tyr; a non-natural analog of phenylalanine, tryptophan, or tyrosine, a hydratable carbonyl-containing residue, or a boronic acid-containing residue;
X11 is Cys, Gln, Lys or Arg;
X12 is Ser, Cys, Gly, 7-aminoheptanoic acid, β-alanine, diaminopropionic acid, propargylglycine, isoaspartic acid, or null;
R¹ is null, -L^{10A}-L^{10B}-R¹⁰, an amino acid peptide sequence optionally substituted with -L^{10A}-L^{10B}-R¹⁰;
R² is null, -L^{20A}-L^{20B}-R²⁰, an amino acid peptide sequence optionally substituted with -L^{20A}-L^{20B}-R²⁰;
wherein
L^{10A}, L^{10B}, L^{20A}, L^{20B} are independently a bond, a peptidyl linker, —O—, —S—,
—C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
R¹⁰ and R²⁰ are independently a reactive moiety, a diagnostic moiety, a therapeutic moiety or a detectable moiety;
wherein X1 and X12 are optionally joined together to form a cyclic peptidyl moiety; and
wherein R¹ and X11 are optionally joined together to form a cyclic peptidyl moiety.

76.-103. (canceled)

104. A peptide compound of formula:
R¹—X0-X1-X2-X3-X4-X5-X6-X7-X8-X9-X10-X11-X12-X13-X14-X15-R² (II)
wherein:
X0 is Ser or null;
X1 is Ser, Cys, Gly, β-alanine, diaminopropionic acid, β-azidoalanine, or null;
X2 is Gln or null;
X3 is Phe, Tyr, β,β′-diphenyl-Ala, His, Asp, 2-bromo-L-phenylalanine, 3-bromo-L-phenylalanine, 4-bromo-L-phenylalanine, Asn, Gln, a modified Phe, a hydratable carbonyl-containing residue, or a boronic acid-containing residue;
X4 is Asp or Asn;
X5 is Leu, β,β′-diphenyl-Ala, Phe, Trp, Tyr, a non-natural analog of phenylalanine, tryptophan, or tyrosine, a hydratable carbonyl-containing residue, or a boronic acid-containing residue;
X6 is Ser;
X7 is Cys, Thr, or Ser;
X8 is Arg, Ala, or an amino acid comprising a side chain of the formula -L^{3A}-L^{3B}-R³, wherein L^{3A} is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, L^{3B} is a chemical linker and R³ is a steric hindering chemical moiety;
X9 is Cys, Arg or Ala;
X10 is Leu, Gln, Glu, β,β′-diphenyl-Ala, Phe, Trp, Tyr; a non-natural analog of phenylalanine, tryptophan, or tyrosine, a hydratable carbonyl-containing residue, or a boronic acid-containing residue;
X11 is Cys, Gln, Lys or Arg;
X12 is Ser, Cys, Gly, 7-aminoheptanoic acid, β-alanine, diaminopropionic acid, propargylglycine, isoaspartic acid, or null;
X13 is Gly or Ser;
X14 and X15 are independently Gly, Ser, Ala, or Cys;
R¹ is null, -L^{10A}-L^{10B}-R¹⁰, an amino acid peptide sequence optionally substituted with -L^{10A}, -L^{10B}-R¹⁰;
R² is null, -L^{20A}-L^{20B}-R²⁰, an amino acid peptide sequence optionally substituted with -L^{20A}-L^{20B}-R²⁰,
wherein
L^{10A}, L^{10B}, L^{20A}, L^{20B} are independently a bond, a peptidyl linker, —O—, —S—,
—C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
R¹⁰ and R²⁰ are independently a reactive moiety, a diagnostic moiety, a therapeutic moiety or a detectable moiety; and
wherein X1 and X12 are optionally joined together to form a cyclic peptidyl moiety.

105.-117. (canceled)

118. An antibody or fragment antigen-binding (Fab) comprising:
(1) a central hole enclosed by the heavy chain variable (VH) region, the light chain variable (VL) region, the heavy chain constant (CH1) region and the light chain constant (CL) region of said antibody or fragment antigen-binding (Fab) between a first cavity and a second cavity; and
(2) a non-CDR peptide binding region comprising:
(a) said first cavity lined by a first set of amino acid residues of the VH, VL, CH1, and CL regions of said body or fragment antigen-binding (Fab), wherein said first set of amino acid residues comprises a cysteine at a position corresponding to Kabat position 102, 142 or 143 of said VL region;
(b) said second cavity lined by a second set of amino acid residues of the VH, VL, CH1, and CL regions of said antibody or fragment antigen-binding (Fab), wherein said second set of amino acid residues comprises a cysteine at a position corresponding to Kabat position 208 or 158 of said VH region; or
(c) a hole region enclosing said hole between said first cavity and said second cavity, said hole region lined by a third set of amino acid residues of the VH, VL, CH1, and CL regions of said antibody or fragment antigen-binding (Fab), wherein said third set of amino acid residues comprises a cysteine at a position corresponding to Kabat position 174 or 175 of said VH region.

119.-128. (canceled)

129. A covalent complex comprising:
(i) an antibody or fragment antigen-binding (Fab) comprising a non-CDR peptide binding region comprising a first cysteine, wherein said first cysteine is at a position corresponding to Kabat position 174 or 175 of said VH region of said an antibody or fragment antigen-binding (Fab); and
(ii) a peptide compound comprising a thiol side chain amino acid covalently bound to said antibody or fragment antigen-binding (Fab) through a disulfide linkage between said first cysteine and said thiol side chain amino acid;
wherein said peptide compound has the formula:
R¹-X0-X1-X2-X3-X4-X5-X6-X7-X8-X9-X10-X11-X12-R² (I)
wherein:
X0 is Ser or null;
X1 is Ser, Cys, Gly, β-alanine, diaminopropionic acid, β-azidoalanine, or null;
X2 is Gln or null;
X3 is Phe, Tyr, β,β′-diphenyl-Ala, His, Asp, 2-bromo-L-phenylalanine, 3-bromo-L-phenylalanine, 4-bromo-L-phenylalanine, Asn, Gln, a modified Phe, a hydratable carbonyl-containing residue, or a boronic acid-containing residue;
X4 is Asp or Asn;
X5 is Leu, β,β′-diphenyl-Ala, Phe, Trp, Tyr, a non-natural analog of phenylalanine, tryptophan, or tyrosine, a hydratable carbonyl-containing residue, or a boronic acid-containing residue;
X6 is said thiol side chain amino acid or serine;
X7 is the thiol side chain amino acid, Thr, or Ser;
X8 is said thiol side chain amino acid, Arg, Ala, or an amino acid comprising a side chain of the formula -L^{3A}-L^{3B}-R³, wherein L^{3A} is a bond, —O—, —S—, —C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene, L^{3B} is a chemical linker and R³ is a steric hindering chemical moiety;
X9 is the thiol side chain amino acid, Arg or Ala;
X10 is Leu, Gln, Glu, β,β′-diphenyl-Ala, Phe, Trp, Tyr; a non-natural analog of phenylalanine, tryptophan, or tyrosine, a hydratable carbonyl-containing residue, or a boronic acid-containing residue;
X11 is the thiol side chain amino acid, Gln, Lys or Arg;
X12 is Ser, Cys, Gly, 7-aminoheptanoic acid, β-alanine, diaminopropionic acid, propargylglycine, isoaspartic acid, or null;
R¹ is null, -L^{10A}-L^{10B}-R¹⁰, an amino acid peptide sequence optionally substituted with -L^{10A}, -L^{10B}-R¹⁰;
R² is null, -L^{20A}-L^{20B}-R²⁰, an amino acid peptide sequence optionally substituted with -L^{20A}-L^{20B}-R²⁰;
wherein
L^{10A}, L^{10B}, L^{20A}, L^{20B} are independently a bond, a peptidyl linker, —O—, —S—,
—C(O)—, —C(O)O—, —C(O)NH—, —S(O)₂NH—, —NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
R¹⁰ and R²⁰ are independently a reactive moiety, a diagnostic moiety, a therapeutic moiety or a detectable moiety;
wherein X1 and X12 are optionally joined together to form a cyclic peptidyl moiety; and
wherein R¹ and X11 are optionally joined together to form a cyclic peptidyl moiety.
